(19) United States
(12) Patent Application Publication
(10) Pub. No.: US 2007/0166704 A1
(43) Pub. Date: Jul. 19, 2007

(54) Identification of polynucleotides and polypeptide for predicting activity of compounds that interact with protein tyrosine kinases and/or protein tyrosine kinase pathways

(76) Inventors: Fei Huang, Princeton, NJ (US); Craig R. Fairchild, Yardley, PA (US); Francis Y. Lee, Yardley, PA (US); Peter Shaw, Yardley, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 10/348,119

(22) Filed: Jan. 17, 2003

Related U.S. Application Data
(60) Provisional application No. 60/350,061, filed on Jan. 18, 2002.

(51) Int. Cl. C12Q 1/68; C07H 21/04

(52) U.S. Cl. 435/6; 536/24.3

(57) ABSTRACT

The present invention describes polynucleotides and polypeptides that have been discovered to correlate to the relative intrinsic sensitivity or resistance of cells, e.g., colon cell lines, to treatment with compounds that interact with and inhibit src tyrosine kinases. These polynucleotides and polypeptides have been shown, through a weighted voting cross validation program, to have utility in predicting the intrinsic resistance and sensitivity of colon cell lines to these compounds. Such polynucleotides and polypeptides whose expression levels correlate highly with drug sensitivity or resistance comprise predictor or marker sets of polynucleotides and polypeptides that are useful in methods of predicting drug response and as prognostic or diagnostic indicators in disease management, particularly in those disease areas in which signaling through src tyrosine kinase of the src tyrosine kinase pathway is involved with the disease process.

This application claims benefit to provisional application U.S. Ser. No. 60/350,061. filed Jan. 18, 2002. The entire teachings. of the referenced application are incorporated herein by reference.

### FIELD OF THE INVENTION

The present invention relates generally to the field of pharmacogenomics, and more specifically to new and alternative methods and procedures to determine drug sensitivity in patients to allow the development of individualized genetic profiles which aid in treating diseases and disorders based on patient response at a molecular level.

### BACKGROUND OF THE INVENTION

The major goal of pharmacogenomics research is to identify genetic markers that accurately predict a given patient's response to drugs in the clinic; such individualized genetic assessment would greatly facilitate personalized treatment. An approach of this nature is particularly needed in cancer treatment and therapy, where commonly used agents are ineffective in many patients, and side effects are frequent.

The classification of patient samples is a crucial aspect of cancer diagnosis and treatment. The association of a patient's response to drug treatment with molecular and genetic markers can open up new opportunities for drug development in non-responding patients, or distinguish a drug's indication among other treatment choices because of higher confidence in the efficacy. Further, the pre-selection of patients who are likely to respond well to a medicine, drug, or combination therapy may reduce the number of patients needed in a clinical study or accelerate the time needed to complete a clinical development program (M. Cockett et al., 2000, Current Opinion in Biotechnology, 11:602-609).

The ability to predict drug sensitivity in patients is particularly challenging because drug responses reflect not only properties intrinsic to the target cells, but also a host's metabolic properties. Efforts by those in the art to use genetic information to predict drug sensitivity have primarily focused on individual polynucleotides and polypeptides that have broad effects, such as the multidrug resistant polynucleotides and polypeptides, mdr1 and mrp1 (P. Sonneveld, 2000, J. Intern. Med., 247:521-534). Microarray technologies have also made it more straightforward to monitor simultaneously the expression pattern of thousands of polynucleotides and polypeptides, to analyze multiple markers and to categorize cancers into subgroups (J. Khan et al., 1998, Cancer Res., 58:5009-5013; A. A. Alizadeh et al., 2000, Nature, 403:503-511; M. Bittner et al., 2000, Nature, 406:536-540; J. Khan et al., 2001, Nature Medicine, 7(6):673-679; and T. R. Golub et al., 1999, Science, 286:531-537).

Such technologies and molecular tools have made it possible to monitor the expression level of a large number of transcripts within a cell at any one time (see, e.g., Schena et al., 1995, Quantitative monitoring of gene expression patterns with a complementary DNA micro-array, Science, 270:467-470; Lockhart et al., 1996, Expression monitoring by hybridization to high-density oligonucleotide arrays, Nature Biotechnology, 14:1675-1680; Blanchard et al., 1996, Sequence to array: Probing the genome's secrets, Nature Biotechnology, 14:1649; U.S. Pat. No. 5,569,588, issued Oct. 29, 1996 to Ashby et al.) In organisms, including humans, for which the complete genome is known, it is possible to analyze the transcripts of all polynucleotides and polypeptides within the cell.

How differential gene expression is associated with health and disease is a basis of functional genomics, which is defined as the study of all of the polynucleotides and polypeptides expressed by a specific cell or a group of cells and the changes in their expression pattern during development, disease, or environmental exposure. Hybridization arrays, used to study gene expression, allow gene expression analysis on a genomic scale by permitting the examination of changes in expression of literally thousands of polynucleotides and polypeptides at one time. In general, for hybridization arrays, gene-specific sequences (probes) are immobilized on a solid state matrix. These sequences are then queried with labeled copies of nucleic acids from biological samples (targets). The underlying theory is that the greater the expression of a gene, the greater the amount of labeled target and thus, the greater output of signal. (W. M. Freeman et al., 2000, BioTechniques), 29:1042-1055).

Recent studies have demonstrated that gene expression information generated by microarray analysis of human tumors can predict clinical outcome (L. J. van't Veer et al., 2002, Nature, 415:530-536; M. West et al., 2001, Proc. Natl. Acad. Sci. USA, 98:11462-11467; T. Sorlie et al., 2001, Proc. Natl. Acad. Sci. USA, 98:10869-10874; M. Shipp et al., 2002, Nature Medicine, 8(1):68-74). These findings bring hope that cancer treatment will be vastly improved by better predicting the response of individual tumors to therapy.

Needed in the art are new and alternative methods and procedures to determine drug sensitivity in patients to allow the development of individualized genetic profiles which aid in treating diseases and disorders based on patient response at a molecular level. By using cultured cells as a model of in vivo effects, the present invention advantageously focuses on cell-intrinsic properties that are exposed in cell culture and involves identified polynucleotides and polypeptides that correlate with drug sensitivity. The presently described discovery and identification of polynucleotides and polypeptides/marker polynucleotides and polypeptides (predictor polynucleotides, predictor polypeptides, predicter polynucleotide subsets, and predictor polypeptide subsets) in cell lines assayed in vitro can be used to correlate with drug responses in vivo, and thus can be extended to clinical situations in which the same polynucleotides and polypeptides are used to predict responses to drugs and/or chemotherapeutic agents by patients.

### SUMMARY OF THE INVENTION

The present invention describes the identification of marker polynucleotides and polypeptides whose expression levels are highly correlated with drug sensitivity in colon cell lines that are either sensitive or resistant to protein tyrosine kinase inhibitor compounds. More particularly, the protein tyrosine kinases that are inhibited in accordance with the present invention include members of the Src family of tyrosine kinases, for example, Src, Fgr, Fyn, Yes, Blk, Hck, Lck and Lyn, as well as other protein tyrosine kinases, including, Bcr-abl, Jak, PDGFR, c-kit and Ephr. For a review of these and other protein tyrosine kinases, see, for example, P. Blume-Jensen and T. Hunter, 2001, “Oncogene Kinase Signaling”, Nature, 411:355-365. Some of these polynucleotides and polypeptides are also modulated by the tyrosine kinase inhibitor compounds, in particular, src tyrosine kinase inhibitor compounds, which indicates their involvement in the protein tyrosine kinase signaling pathway. These polynucleotides and polypeptides or “markers” show utility in predicting a host's response to a drug and/or drug treatment.

It is an object of this invention to provide a cell culture model to identify polynucleotides and polypeptides whose expression levels correlate with drug sensitivity of cells associated with a disease state, or a host having a disease. In accordance with the present invention, oligonucleotide microarrays were utilized to measure the expression levels of a large number of polynucleotides and polypeptides in a panel of untreated cell lines, particularly colon cell lines, for which drug sensitivity to four src kinase inhibitor compounds was determined. The determination of the gene expression profiles in the previously untreated cells allowed a prediction of chemosensitivity and the identification of marker polynucleotides and polypeptides whose expression levels highly correlate with sensitivity to drugs or compounds that modulate, preferably inhibit, src kinase or src family kinases or the pathway in which src or src family tyrosine kinases are involved. The marker or predictor polynucleotides and polypeptides are thus useful for predicting a patient's response to drugs or drug treatments that directly or indirectly affect src or src family tyrosine kinases activity.

It is another object of the present invention to provide a method of determining or predicting if an individual requiring drug or chemotherapeutic treatment or therapy for a disease state, for example, colon disease, or a cancer or tumor of a particular type, preferably, a colon cancer or tumor, will successfully respond or will not respond to the drug or chemotherapeutic treatment or therapy, preferably a treatment or therapy involving a src or src family tyrosine kinases modulating agent, e.g., an inhibitor of src kinase activity, prior to subjecting the individual to such treatment or chemotherapy. Preferably, the treatment or therapy involves a protein tyrosine kinase modulating agent, e.g., an inhibitor of the protein tyrosine kinase activity. The protein tyrosine kinases whose activities can be inhibited by inhibitor compounds according to this invention include, for example, members of the Src family of tyrosine kinases, for example, Src, Fgr, Fyn, Yes, Blk, Hck, Lck and Lyn, as well as other protein tyrosine kinases, including, Bcr-abl, Jak, PDGFR, c-kit and Ephr. In accordance with the present invention, cells from a patient tissue sample, e.g., a tumor or cancer biopsy, preferably a colon cancer or tumor sample, or sloughed colonocytes, are assayed to determine their gene expression pattern prior to treatment with a src or src family tyrosine kinases modulating compound or drug, preferably a src kinase inhibitor. The resulting gene expression profile of the test cells before exposure to the compound or drug is compared with the gene expression pattern of the predictor set of polynucleotides and polypeptides that have been described and shown herein (Tables 3-6) in the control panel of the untreated cells that are either resistant or sensitive to the drug or compound, i.e., FIGS. 1-3.

In addition, in such a method, the gene expression pattern of subsets of predictor polynucleotides and polypeptides, comprising at least about 5, at least about 10, at least about 15, at least about 20, at least about 25, at least about 30, at least about 40, at least about 45 at least about 50, or more, polynucleotides and polypeptides may be used. In this context, the term “about” may be construed to mean 1, 2, 3, 4, or 5 more or less polynucleotides or polypeptides within each predicter subset. Preferably, in such a method, the gene expression pattern of subsets of predictor polynucleotides and polypeptides, comprising sets of 25, 15 and 10 polynucleotides and polypeptides as set forth in Tables 10 thru 12, respectively, can also be used. These polynucleotides and polypeptides are derived from the control panel of the untreated cells that have been determined to be either resistant or sensitive to the drug or compound as shown herein.

Success or failure of treatment with a drug can be determined based on the gene expression pattern of the test cells from the test tissue, e.g., tumor or cancer biopsy, as being relatively the same as or different from the gene expression pattern of the predictor set of polynucleotides and polypeptides in the resistant or sensitive control panel of cells for which drug sensitivity to the src kinase inhibitor compounds has been determined. Thus, if the test cells show a gene expression profile which corresponds to that of the predictor set of polynucleotides and polypeptides in the control panel of cells which are sensitive to the drug or compound, it is highly likely or predicted that the individual's cancer or tumor will respond favorably to treatment with the drug or compound. By contrast, if the test cells show a gene expression pattern corresponding to that of the predictor set of polynucleotides and polypeptides of the control panel of cells which are resistant to the drug or compound, it is highly likely or predicted that the individual's cancer or tumor will not respond to treatment with the drug or compound.

It is an aspect of this invention to provide screening assays for determining if a cancer patient will be susceptible or resistant to treatment with a drug or compound, particularly, a drug or compound directly or indirectly involved in a protein tyrosine kinase activity or a protein tyrosine kinase pathway. Such protein tyrosine kinases include, without limitation, members of the Src family of tyrosine kinases, for example, Src, Fgr, Fyn, Yes, Blk, Hck, Lck and Lyn, as well as other protein tyrosine kinases, including, Bcr-abl, Jak, PDGFR, c-kit and Ephr.

It is a further object of the present invention to provide screening assays for determining if a patient's cancer tumor will be susceptible or resistant to treatment with a drug or compound, particularly, a drug or compound directly or indirectly involved in src or src family tyrosine kinases activity or the src or src family tyrosine kinases pathway.

It is another object of the present invention to provide a method of monitoring the treatment of a patient having a disease treatable by a compound or agent that modulates a src tyrosine kinase by comparing the resistance or sensitivity gene expression profile of cells from a patient tissue sample, e.g., a tumor or cancer biopsy, preferably a colon cancer or tumor sample, prior to treatment with a drug or compound that inhibits src or src family tyrosine kinases activity and again following treatment with the drug or compound. The isolated cells from the patient are assayed to determine their gene expression pattern before and after exposure to a compound or drug, preferably a src kinase inhibitor, to determine if a change of the gene expression profile has occurred so as to warrant treatment with another drug or agent, or to discontinue current treatment. The resulting gene expression profile of the cells tested before and after treatment is compared with the gene expression pattern of the predictor set of polynucleotides and polypeptides that have been described and shown herein to be highly expressed in cells that are either resistant or sensitive to the drug or compound.

Such a monitoring process can indicate success or failure of a patient's treatment with a drug or compound based on the gene expression pattern of the cells isolated from the patient's sample, e.g., a tumor or cancer biopsy, as being relatively the same as or different from the gene expression pattern of the predictor gene set of the resistant or sensitive control panel of cells that have been exposed to the drug or compound and assessed for their gene expression profile following exposure. Thus, if, after treatment with a drug or compound, the test cells show a change in their gene expression profile from that seen prior to treatment to one which corresponds to that of the control panel of cells that are resistant to the drug or compound, it can serve as an indicator that the current treatment should be modified, changed, or even discontinued. Also, should a patient's response become one that is sensitive to treatment by a src kinase inhibitor compound, based on correlation of the expression profile of the predictor polynucleotides and polypeptides, the patient's treatment prognosis can be qualified as favorable and treatment can continue. Such monitoring processes can be repeated as necessary or desired. The monitoring of a patient's response to a given drug treatment can also involve testing the patient's cells in the assay as described only after treatment, rather than before and after treatment, with drug or active compound.

It is a further object of the present invention to provide predictor polynucleotides and polypeptides and predictor sets of polynucleotides and polypeptides as tools that have both diagnostic and prognostic value in disease areas in which signaling through protein tyrosine kinase or a protein tyrosine kinase pathway is of importance, e.g., in cancers and tumors, in immunological disorders, conditions or dysfunctions, or in disease states in which cell signaling and/or proliferation controls are abnormal or aberrant. Such protein tyrosine kinases whose direct or indirect modulation can be associated with a disease state or condition, include members of the Src family of tyrosine kinases, for example, Src, Fgr, Fyn, Yes, Blk, Hck, Lck and Lyn, as well as other protein tyrosine kinases, including, Bcr-abl, Jak, PDGFR, c-kit and Ephr. In accordance with this invention, the use of predictor polynucleotides and polypeptides, or a predictor gene set, is to forecast or foretell an outcome prior to having any knowledge about a biological system, or a cellular response. Also according to this invention, the predictor polynucleotides and polypeptides or predictor gene set is useful in predicting the phenotype that is used to classify a biological system or response. For example, the classification of a cell line as “resistant” or “sensitive” is based on the log₁₀(IC₅₀) value of each cell line to one or more compounds (e.g., a src kinase inhibitor compound), relative to the mean log₁₀(IC₅₀) value of a cell line panel (e.g., a thirty-one colon cell line panel, as described herein) that has been previously exposed to the compounds and statistically assessed as to the expression level of polynucleotides and polypeptides correlating to resistance or sensitivity following exposure to the one or more compounds.

It is yet another object of the present invention to provide polynucleotides and polypeptides, such as those listed in Tables 3-5, or the common polynucleotides and polypeptides shown in Table 6 herein, to assemble predictor gene subsets such as in Tables 10-12 to be able to predict or reasonably foretell the likely effect of either src tyrosine inhibitor compounds or compounds that affect the src tyrosine kinase signaling pathway in different biological systems, or for cellular responses. The predictor gene sets can be used in in vitro assays of drug response by test cells to predict in vivo outcome. In accordance with this invention, the various predictor gene sets described herein, or the combination of these predictor sets with other polynucleotides and polypeptides or other co-variants of these polynucleotides and polypeptides, can be used, for example, to predict how patients with cancer or a tumor might respond to therapeutic intervention with compounds that modulate the src tyrosine kinase family. In addition, such predictor sets can be used to predict how patients might respond to therapeutic intervention(s) that modulate(s) signaling through the entire src tyrosine kinase regulatory pathway. The predictor sets of polynucleotides and polypeptides, or co-variants of these polynucleotides and polypeptides, can be used to predict how patients with a cancer or tumor respond to therapy employing compounds that modulate a tyrosine kinase, or the activity of a tyrosine kinase, such as protein tyrosine kinase members of the Src family, for example, Src, Fgr, Fyn, Yes, Blk, Hck, Lck and Lyn, as well as other protein tyrosine kinases, including, Bcr-abl, Jak, PDGFR, c-kit and Ephr.

A further object of the present invention is to provide polynucleotides and polypeptides comprising one or more predictor sets of polynucleotides and polypeptides that most highly correlate with resistance or sensitivity to drugs or compounds which are directly or indirectly involved with modulation of src tyrosine kinase or src tyrosine kinase signaling pathways. In accordance with this invention, predictor gene sets associated with resistance or sensitivity to src tyrosine kinase inhibitor compounds comprise the polynucleotides and polypeptides presented in FIGS. 1-3 and Tables 3-6 herein. Also according to the invention, the polynucleotides and polypeptides of Tables 3-6 have been discovered to be expressed by cells which are sensitive or resistant to four different src kinase inhibitor compounds. The expression of these polynucleotides and polypeptides, or combinations thereof, has been found to be highly correlated with sensitivity of cells to the different src kinase inhibitors. The expression patterns of the three sets of polynucleotides and polypeptides correlating with sensitivity of thirty-one colon cells to the src kinase inhibitor compounds are provided in FIGS. 1-3.

Yet another object of the present invention is to provide predictor polynucleotides and polypeptides or predictor gene sets having both diagnostic and prognostic value in disease areas in which signaling through src tyrosine kinase or the src tyrosine kinase pathway is involved, e.g., in cancers or tumors, or in disease states in which cell signaling and/or cellular proliferation controls are abnormal or aberrant. Also provided by this invention are common polynucleotides and polypeptides whose expression levels are strongly correlated with either sensitivity or resistance to all four of the src kinase inhibitor compounds (Table 6). Because these polynucleotides and polypeptides correlate to drug sensitivity and resistance classifications associated with all four of the src kinase inhibitor compounds in cells, such polynucleotides and polypeptides can be used to build predictors or markers for other biological systems in which src kinase activity or src or src family tyrosine kinases signaling pathways are involved.

Another object of the present invention is to provide one or more specialized microarrays, e.g., oligonucleotide microarrays or cDNA microarrays, comprising those polynucleotides and polypeptides, or combinations thereof, as described herein showing expression profiles that correlate with either sensitivity or resistance to one or more src kinase inhibitor compounds. Such microarrays can be employed in in vitro assays for assessing the expression level of the polynucleotides and polypeptides on the microarrays in the test cells from tumor biopsies, for example, and determining whether these test cells will be likely to be resistant or sensitive to src kinase inhibitor compounds. For example, one or more microarrays can be prepared using each of the polynucleotides and polypeptides, or combinations thereof, as described herein and shown in FIGS. 1-3 and Tables 3-6. Cells from a tissue or organ biopsy can be isolated and exposed to one or more of the inhibitor compounds.

Following application of nucleic acids isolated from both untreated and treated cells to one or more of the specialized microarrays, the pattern of gene expression of the tested cells can be determined and compared with that of the predictor gene pattern from the panel of cells used to create the predictor gene set on the microarray. Based upon the gene expression pattern results from the cells undergoing testing, it can be determined if the cells show a resistant or a sensitive profile of gene expression. Whether or not the tested cells from a tissue or organ biopsy will respond to one or more of the inhibitor compounds and the course of treatment or therapy can then be determined or evaluated based on the information gleaned from the results of the specialized microarray analysis.

It is a further object of the present invention to provide a kit for determining or predicting drug susceptibility or resistance by a patient having a disease, with particular regard to a cancer or tumor, namely, a colon cancer or tumor. Such kits would be useful in a clinical setting for use in testing patient's biopsied tumor or cancer samples, for example, to determine or predict if the patient's tumor or cancer will be resistant or sensitive to a given treatment or therapy with a drug, compound, chemotherapy agent, or biological agent that are directly or indirectly involved with modification, preferably, inhibition, of src tyrosine kinase activity or a cell signaling pathway involving src tyrosine kinase activity. Provided in the kit are one or more predictor gene sets, preferably comprising one or more microarrays, e.g., oligonucleotide microarrays or cDNA microarrays, comprising those polynucleotides and polypeptides that correlate with resistance and sensitivity to Src family of protein tyrosine kinases, for example, Src, Fgr, Fyn, Yes, Blk, Hck, Lck and Lyn, as well as inhibitors of the Bcr-abl, Jak, PDGFR, c-kit and Ephr protein tyrosine kinases; and, in suitable containers, the modulator agents/compounds for use in testing cells from patient tissue specimens or patient samples for resistance/sensitivity to compounds that inhibit src or src family tyrosine kinases activity; and instructions for use. In addition, kits contemplated by the present invention also include reagents or materials for the monitoring of the expression of the predictor or marker polynucleotides and polypeptides of the invention at the level of mRNA or protein, using other techniques and systems practiced in the art, e.g., RT-PCR assays, which employ primers designed on the basis of one or more of the predictor polynucleotides and polypeptides described herein, immunoassays, such as enzyme linked immunosorbent assays (ELISAs), immunoblotting, e.g., Western blots, or in situ hybridization, and the like, as further described herein.

Another object of the present invention is to provide one or more polynucleotides and polypeptides among those of the predictor polynucleotides and polypeptides identified herein that can serve as targets for the development of drug therapies for disease treatment. Such targets may be particularly applicable to treatment of colon disease, such as colon cancers or tumors. Because these predictor polynucleotides and polypeptides are differentially expressed in sensitive and resistant cells, their expression pattern is correlated with the relative intrinsic sensitivity of cells to treatment with compounds that interact with and/or inhibit protein tyrosine kinases, including members of the Src family of protein tyrosine kinases, for example, Src, Fgr, Fyn, Yes, Blk, Hck, Lck and Lyn, as well as the Bcr-abl, Jak, PDGFR, c-kit and Ephr protein tyrosine kinases. Accordingly, the polynucleotides and polypeptides highly expressed in resistant cells can serve as targets for the development of new drug therapies for those tumors which are resistant to protein tyrosine kinase inhibitor compounds.

Yet another object of the present invention is to provide antibodies, either polyclonal or monoclonal, directed against one or more of the src biomarker polypeptides, or peptides thereof, encoded by the predictor polynucleotides and polypeptides. Such antibodies can be used in a variety of ways, for example, to purify, detect, and target the src biomarker polypeptides of the present invention, including both in vitro and in vivo diagnostic, detection, screening, and/or therapeutic methods, and the like. Included among the protein tyrosine kinase biomarker polypeptides of this invention are members of the Src family of protein tyrosine kinases, for example, Src, Fgr, Fyn, Yes, Blk, Hck, Lck and Lyn, as well as the Bcr-abl, Jak, PDGFR, c-kit and Ephr protein tyrosine kinases.

Further objects, features, and advantages of the present invention will be better understood upon a reading of the detailed description of the invention when considered in connection with the accompanying figures or drawings.

### DESCRIPTION OF THE FIGURES

The file of this patent contains at least one Figure executed in color. Copies of this patent with color Figure(s) will be provided by the Patent and Trademark Office upon request and payment of the necessary fee.

FIG. 1 illustrates a gene expression pattern according to the present invention. The 123 polynucleotides and polypeptides that most highly correlated with a resistance/sensitivity phenotype classification of the 31 colon cell lines for BMS-A or BMS-D are shown. Each row corresponds to a gene, with the columns corresponding to expression levels in the different cell lines. Expression levels for each gene are normalized across the median expression level of all the 31 cell lines. The polynucleotides and polypeptides with expression levels greater than the median are shaded in red, and those below the median are shaded in green. The individual polynucleotides and polypeptides encoding the src biomarkers of the FIG. 1 are in the order as listed in Table 3.

FIG. 2 illustrates a gene expression pattern according to the present invention. The 119 polynucleotides and polypeptides most highly correlated with a resistance/sensitivity phenotype classification of the 31 colon cell lines for BMS-B are shown. Each row corresponds to a gene, with the columns corresponding to expression levels in the different cell lines. Expression levels for each gene are normalized across the median expression level of all the 31 cell lines. The polynucleotides and polypeptides with expression levels greater than the median are shaded in red, and those below the median are shaded in green. The individual polynucleotides and polypeptides encoding the src biomarkers of the FIG. 2 are in the order as listed in Table 4.

FIG. 3 illustrates a gene expression pattern according to the present invention. The 137 polynucleotides and polypeptides most highly correlated with a resistance/sensitivity phenotype classification of the 31 colon cell lines for BMS-C are shown. Each row corresponds to a gene, with the columns corresponding to expression levels in the different cell lines. Expression levels for each gene are normalized across the median expression level of all the 31 cell lines. The polynucleotides and polypeptides with expression levels greater than the median are shaded in red, and those below the median are shaded in green. The individual polynucleotides and polypeptides encoding the src biomarkers of the FIG. 3 are in the order as listed in Table 5.

FIG. 4 shows the error rates of prediction for the four src kinase inhibitor compounds, BMS-A, BMS-B, BMS-C and BMS-D in cross validation and random permutation tests. The Genecluster software was used to select polynucleotides and polypeptides and predict classifications using a “weighted-voting ‘leave one out’ cross-validation algorithm”, as described herein. A different number of polynucleotides and polypeptides was used in the predictor set for predicting resistant and sensitive classes to BMS-A, BMS-B, BMS-C and BMS-D in the colon cell lines. The real error rates were compared with the real error rates using the same number of polynucleotides and polypeptides as the predictor set in 20 cases, in which classification for the colon cell lines was randomly assigned. For example, when each predictor set contained 20 polynucleotides and polypeptides, the real error rate of prediction for BMS-A or BMS-D was 15.7%; for BMS-B and BMS-C, the real error rates were 19% and 16.2%, respectively. These error rate values are significantly lower than the real error rates obtained when random phenotype classifications are used for the cell lines (i.e., in a range of from 30% to 70%).

### DESCRIPTION OF THE TABLES

Table 1 shows the mean IC₅₀ of four src kinase inhibitors for each of the thirty-one colon cell lines. Thirty-one colon cell lines were treated with each of the four src tyrosine kinase inhibitor compounds, namely, BMS-A, BMS-B, BMS-C and BMS-D, and the IC₅₀ was assessed in the cells by MTS assays as described in Example 1 (Methods). The mean IC₅₀ values along with standard deviations (SD) were calculated from 2 to 5 individual determinations for each cell line for the results shown. The IC₅₀ unit is μM.

Table 2 shows the resistance/sensitivity classification of 31 colon cell lines for the four src kinase inhibitor compounds BMS-A, BMS-B, BMS-C and BMS-D. For each compound, the IC₅₀ for each cell line was log-transformed to log₁₀(IC₅₀), and the log₁₀(IC₅₀) values were then normalized to the mean log₁₀(IC₅₀) across the 31 colon cell lines. The cell lines with log₁₀(IC₅₀) below the mean log₁₀(IC₅₀) of all 31 cell lines were defined as sensitive to the compound, while those with log₁₀(IC₅₀) above the mean log₁₀(IC₅₀) were considered to be resistant.

Table 3 shows a gene list that demonstrated a high correlation between expression pattern and resistance/sensitivity classification to BMS-A or BMS-D. The gene number, relative expression pattern, i.e., sensitive or resistant, Gene Accession number, gene description (Unigene cluster), SEQ ID NO: for the DNA sequence of the gene, and SEQ ID NO: for the amino acid sequence of the gene (if available), are presented in the table. For each gene, the DNA and encoded amino acid sequences represented by SEQ ID NOs. in the table are described in the Sequence Listing.

Table 4 presents a gene list that demonstrated high correlation between expression pattern and resistance/sensitivity classification to BMS-B. The gene number, relative expression pattern, i.e., sensitive or resistant, Gene Accession number, gene description (unigene cluster), SEQ ID NO: for the DNA sequence of the gene, and SEQ ID NO: for the amino acid sequence of the gene (if available), are presented in the table. For each gene, the DNA and encoded amino acid sequences represented by SEQ ID NOs. in the table are described in the Sequence Listing.

Table 5 presents a gene list that demonstrated high correlation between expression pattern and resistance/sensitivity classification to BMS-C. The gene number, relative expression pattern, i.e., sensitive or resistant, Gene Accession number, gene description (unigene cluster), SEQ ID NO: for the DNA sequence of the gene, and SEQ ID NO: for the amino acid sequence of the gene (if available), are presented in the table. For each gene, the DNA and encoded amino acid sequences represented by SEQ ID NOs. in the table are described in the Sequence Listing.

Table 6 presents a common gene list from Tables 3-5 showing the highest correlation between expression pattern and resistance/sensitivity classification of the cells to the four src kinase inhibitor compounds BMS-A/BMS-D, BMS-B and BMS-C. The gene description, accession number, DNA sequence, amino acid sequence (if available), and the corresponding nucleic acid and amino acid SEQ ID NOS are provided. The relative expression patterns of each gene i.e., sensitive or resistant, are indicated.

Table 7 presents a resistance/sensitivity prediction of the 31 colon cell lines for BMS-A or BMS-D, BMS-B and BMS-C using 10 markers as a predictor set shown in Table 10. The true class is assigned as in Table 2, based on the IC₅₀ results. The predicted class is determined by using the optimal 10 polynucleotides and polypeptides as the predictor set to predict the resistance or sensitive class. “S” represents Sensitive; “R” represents Resistant. The confidence score refers to prediction strength for each prediction made on a cell line by the predictor set. The confidence score ranges from 0 to 1, i.e., corresponding from low to high confidence in making the prediction. The error predictions are indicated by an asterisk (*).

Table 8 shows a resistance/sensitivity prediction of the 31 colon cell lines for or BMS-D, BMS-B and BMS-C using 15 markers as a predictor set shown in Table 11. The true class is assigned as in Table 2, based on the IC₅₀ results. The predicted class is determined by using the optimal 15 polynucleotides and polypeptides as the predictor set to predict the resistance or sensitive class. “S” represents Sensitive; “R” represents Resistant. The confidence score refers to prediction strength for each prediction made on a cell line by the predictor set. The confidence score ranges from 0 to 1, i.e., corresponding from low to high confidence in making the prediction. The error predictions are indicated by an asterisk (*).

Table 9 presents a resistance/sensitivity prediction of the 31 colon cell lines for BMS-A or BMS-D, BMS-B and BMS-C using 25 markers as a predictor set shown in Table 12. The true class is assigned as in Table 2, based on the IC₅₀ results. The predicted class is determined by using the optimal 25 polynucleotides and polypeptides as the predictor set to predict the resistance or sensitive class. “S” represents Sensitive; “R” represents Resistant. The confidence score refers to prediction strength for each prediction made on a cell line by the predictor set. The confidence score ranges from 0 to 1, i.e., corresponding from low to high confidence in making the prediction. The error predictions are indicated by an asterisk (*).

Table 10 lists the predictor set of 10 polynucleotides and polypeptides used in prediction as shown in Table 7. These 10 polynucleotides and polypeptides were selected from the 73 common (as shown in Table 6). Gene Accession number, gene description (Unigene cluster), and relative expression pattern, i.e., sensitive or resistant, for this 10-gene predictor subset, are indicated.

Table 11 lists the predictor set of 15 polynucleotides and polypeptides used in prediction as shown in Table 8. These 15 polynucleotides and polypeptides were selected from the 73 common (as shown in Table 6). Gene Accession number, gene description (Unigene cluster), and relative expression pattern, i.e., sensitive or resistant, for this 15-gene predictor subset, are indicated.

Table 12 lists the predictor set of 25 polynucleotides and polypeptides used in prediction as shown in Table 9. These 25 polynucleotides and polypeptides were selected from the 73 common (as shown in Table 6). Gene Accession number, gene description (Unigene cluster), and relative expression pattern, i.e., sensitive or resistant, for this 25-gene predictor subset, are indicated.

Table 13 show representative forward and reverse RT-PCR primers for each of the Src biomarker polynucleotides and polypeptides of the present invention, as identified by SEQ ID NO and Accession No. in Tables 3-5.

### DETAILED DESCRIPTION OF THE INVENTION

The present invention describes the identification of polynucleotides and polypeptides that correlate with drug sensitivity or resistance employing cell lines that are previously untreated with drug to determine sensitivity of the cells to a drug, compound, or biological agent. These polynucleotides and polypeptides, called marker or predictor polynucleotides and polypeptides herein, can be employed for predicting drug response. The marker polynucleotides and polypeptides have been determined in an in vitro assay employing microarray technology to monitor simultaneously the expression pattern of thousands of discrete polynucleotides and polypeptides in previously untreated cells, whose sensitivity to compounds or drugs, in particular, compounds that inhibit protein tyrosine kinase or protein tyrosine kinase activity, particularly src or src family tyrosine kinases, is tested. The protein tyrosine kinases, or activities thereof, associated with response to a drug, compound, or biological agent include, for example, members of the Src family of protein tyrosine kinases, for example, Src, Fgr, Fyn, Yes, Blk, Hck, Lck and Lyn, as well as the Bcr-abl, Jak, PDGFR, c-kit and Ephr protein tyrosine kinases. (See, e.g., P. Blume-Jensen and T. Hunter, 2001, “Oncogene Kinase Signaling”, Nature, 411:355-365).

This assay has allowed the identification of the marker polynucleotides and polypeptides, called src biomarkers herein, having expression levels in the cells that are highly correlated with drug sensitivity exhibited by the cells. Such marker polynucleotides and polypeptides serve as useful molecular tools for predicting a response to drugs, compounds, biological agents, chemotherapeutic agents, and the like, preferably those drugs and compounds, and the like, that affect protein tyrosine kinase activity, particularly src or src family tyrosine kinases activity, via direct or indirect inhibition or antagonism of protein tyrosine kinase function, particularly src or src family tyrosine kinases function or activity.

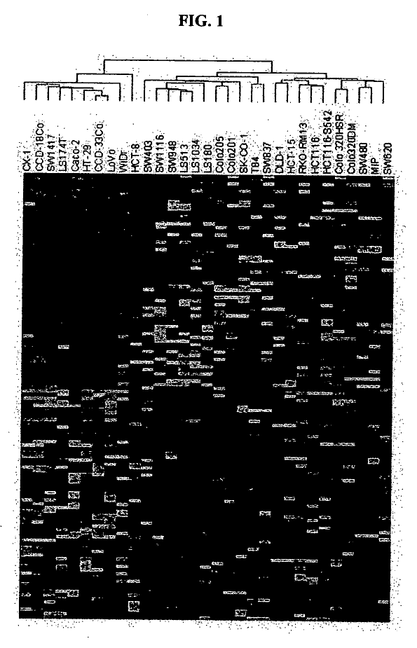

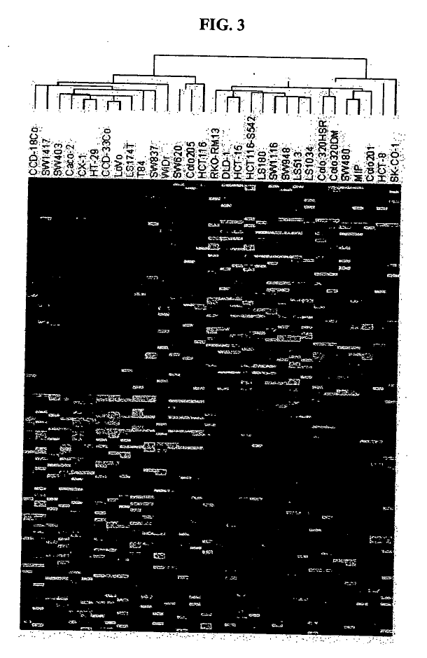

In its preferred aspect, the present invention describes polynucleotides and polypeptides that correlate with sensitivity or resistance of colon cell lines to treatment with protein tyrosine kinase inhibitor compounds, particularly src tyrosine kinase inhibitor compounds as described herein. The exposure of thirty-one colon cell lines to each of four src kinase inhibitor compounds provided a predictor set of polynucleotides and polypeptides for each compound that were most highly correlated with a resistance or sensitivity classification of the thirty-one colon cell lines to the inhibitor compounds. (FIGS. 1-3 and Tables 3-5). The src kinase inhibitor compounds utilized for identifying the gene predictor sets of this invention are described in WO 00/62778, published Oct. 26, 2000. Specifically, for the four src kinase inhibitor compounds analyzed, namely, BMS-A, BMS-B, BMS-C and BMS-D, the drug sensitivity classification for the thirty-one colon cell lines was the same for BMS-A and BMS-D; and 26 out of 31 colon cell lines have the same sensitivity classifications for all four src kinase inhibitor compounds as shown in the Table 2. One or more of these four compounds has a potent inhibitory activity for a number of protein tyrosine kinases, for example, members of the Src family of protein tyrosine kinases, including Src, Fgr, Fyn, Yes, Blk, Hck, Lck and Lyn, as well as the Bcr-abl, Jak, PDGFR, c-kit and Ephr protein tyrosine kinases. Although the predicter gene sets are most useful in predicting efficacy of one or more of these compounds for inhibiting Src kinase function and/or activity specifically, the predicter gene sers are also useful for predicting the efficacy of these compounds for inhibiting protein tyrosine kinases, in general, an in particularly Src, Fgr, Fyn, Yes, Blk, Hck, Lck and Lyn, as well as the Bcr-abl, Jak, PDGFR, c-kit and Ephr protein tyrosine kinases.

The expression of 123, 119 and 137 predictor polynucleotides and polypeptides, was found to correlate with resistance/sensitivity of the colon cell lines to BMS-A/BMS-D, BMS-B and BMS-C respectively. Common predictor polynucleotides and polypeptides were also determined for predicting a resistance/sensitivity classification of cells to the src kinase inhibitors. The common polynucleotides and polypeptides showing the highest correlation between their expression pattern and the resistance or sensitivity classification in the cell lines for the src kinase inhibitor compounds are presented in Table 6.

In accordance with the invention, an approach has been discovered in which polynucleotides and polypeptides and combinations of polynucleotides and polypeptides have been identified whose expression pattern, in a subset of cell lines, correlates to and can be used as an in vitro predictor of cellular response to treatment or therapy with one compound, or with a combination or series of compounds, that are known to inhibit or activate the function of a protein, enzyme, or molecule (e.g., a receptor) that is directly or indirectly involved in cell proliferation, cell responses to external stimuli, (such as ligand binding), or signal transduction, e.g., a tyrosine kinase. Preferred are antagonists or inhibitors of the function of a given protein, e.g., a tyrosine kinase.

In a preferred aspect, specific src tyrosine kinase inhibitor compounds, BMS-A, BMS-B, BMS-C and BMS-D were employed to determine drug sensitivity in a panel of colon cell lines following exposure of the cells to the compounds. Some of the cell lines were determined to be resistant to treatment with the inhibitor compounds, while others were determined to be sensitive to the inhibitors (Tables 1 and 2). A subset of the cell lines examined provided an expression pattern or profile of polynucleotides and polypeptides, and combinations of polynucleotides and polypeptides, that correlated to and serve as a predictor of, a response by the cells to these inhibitor compounds, and to compounds having similar modes of action and/or structure. (FIGS. 1-3 and Tables 7-12).

Such a predictor set of cellular gene expression patterns correlating with sensitivity or resistance of cells following exposure of the cells to a drug, or a combination of drugs, provides a useful tool for screening a tumor sample before treatment with the drug, or a similar drug, or drug combination. The screening technique allows a prediction of cells of a tumor sample exposed to a drug, or a combination of drugs, based on the gene expression results of the predictor set, as to whether or not the tumor, and hence a patient harboring the tumor, will or will not respond to treatment with the drug or drug combination.

In addition, the predictor polynucleotides and polypeptides or predictor gene set can also be utilized as described herein for monitoring the progress of disease treatment or therapy in those patients undergoing treatment for a disease involving a src or src family tyrosine kinases inhibitor compound or chemotherapeutic agent.

According to a particular embodiment of the present invention, oligonucleotide microarrays were utilized to measure the expression levels of over 12,000 polynucleotides and polypeptides in a panel of thirty-one untreated colon cell lines for which the drug sensitivity to four src kinase inhibitor compounds was determined. This analysis was performed to determine whether the gene expression signatures of untreated cells were sufficient for the prediction of chemosensitivity. Data analysis allowed the identification of marker polynucleotides and polypeptides whose expression levels were found to be highly correlated with drug sensitivity. In addition, the treatment of untreated cells with drug also provided gene expression signatures predictive of resistance to the compounds. Subsequent data analysis allowed the identification of marker polynucleotides and polypeptides whose expression levels were found to be highly correlated with drug resistance. Thus, in one of its embodiments, the present invention provides these polynucleotides and polypeptides, or “markers”, or predictors, which show utility in predicting drug response upon treatment or exposure of cells to drug. In particular, the marker or predictor polynucleotides and polypeptides are src biomarker polynucleotides and polypeptides encoding src biomarker proteins/polypeptides.

The means of performing the gene expression and marker gene identification analyses embraced by the present invention is described in further detail and without limitation herein below.

### IC₅₀ Determination and Phenotype Classification Based on Sensitivity of Thirty-One Colon Cell Lines to src Kinase Inhibitor Compounds

Thirty-one colon cell lines were treated with each of four src tyrosine kinase inhibitor compounds (BMS-A, BMS-B, BMS-C and BMS-D) to determine the IC₅₀ value for each cell line. The average IC₅₀ values, along with standard deviations, were calculated from 2 to 5 individual determinations for each cell line. As shown in Table 1, a large variation in the IC₅₀ values (>1000-fold) was observed for these compounds among the thirty-one cell lines.

The IC₅₀ value for each cell line was log₁₀ transformed. The mean of log₁₀(IC₅₀) across the thirty-one colon cell lines was calculated for each compound. The value of log₁₀(IC₅₀) for each cell line was compared to the mean value of log₁₀(IC₅₀) across the thirty-one colon cell lines for each drug. The cell lines with a log₁₀(IC₅₀) below the mean of log₁₀(IC₅₀) were classified as sensitive to the compound, and those with an log₁₀(IC₅₀) above the mean of log₁₀(IC₅₀) were classified as resistant. Table 2 represents the resistance/sensitivity classifications of the thirty-one colon cell lines for BMS-A, BMS-B, BMS-C and BMS-D, respectively.

As demonstrated in Table 2, the drug sensitivity classification for the thirty-one colon cell lines was the same for BMS-A and BMS-D even though the IC₅₀ for these two compounds was not identical for each cell line. It was also demonstrated that most of the cell lines (26 out of 31) had the same resistance/sensitivity classification for all four of the src kinase inhibitor compounds tested. Five cell lines appeared to have different classifications for the four src kinase inhibitor compounds as indicated in the Table 2.

### Identifying Genes that Significantly Correlated with Drug Resistance/Sensitivity Classification

Expression profiling data of 12,558 polynucleotides and polypeptides represented on the HG-U95Av2 array for thirty-one untreated colon cell lines were obtained and preprocessed as described in Example 1, Methods. The preprocessed data were analyzed using the K-mean Nearest Neighborhood (KNN) algorithm to identify polynucleotides and polypeptides whose expression patterns were strongly correlated with the drug resistance/sensitivity classification. (Table 2). An “idealized expression pattern” corresponds to a gene that is uniformly high in one class (e.g., sensitive) and uniformly low in the other class (e.g., resistant). Initially, a KNN analysis was performed in which a correlation coefficient was obtained for each gene. The correlation coefficient, which is a measure of relative classification separation, is obtained using the following formula:
P(g,c)=(μ1−μ2)/(σ1+σ2).

In the above formula, for P(g,c), P represents correlation coefficient; g represents gene expression; and c represents classification.

μ1 represents the mean gene expression level of samples in class 1;

μ2 represents the mean gene expression level of samples in class 2;

σ1 represents the standard deviation of gene expression for samples in class 1; and σ2 represents the standard deviation of gene expression for samples in class 2

Large values of P(g,c) indicate a strong correlation between gene expression and resistance/sensitivity classification. When the correlation is compared with that in a random permutation test (randomly assigned classification), a significance measurement is obtained. Then, the polynucleotides and polypeptides can be ranked according to the correlation coefficient obtained from this analysis, with the highest value indicating the best correlation of gene expression level with the resistance/sensitivity classification to the src kinase inhibitor compounds in the thirty-one colon cell lines.

The KNN analysis demonstrated that many polynucleotides and polypeptides correlated with the drug resistance/sensitivity classification for all four of the test compounds. Therefore, for greater stringency, two different methods were applied to select a smaller subset of polynucleotides and polypeptides that correlated with the drug resistance/sensitive classification for all of the compounds:

First, a permutation test was performed to calculate the significance of the correlation coefficients obtained in the above-described KNN analysis for the top 200 polynucleotides and polypeptides. Those polynucleotides and polypeptides whose ‘p’ value was less than or equal to 0.05 were selected. Second, a T-test was performed and those polynucleotides and polypeptides with a ‘p’ value that was equal to or less than 0.05 were selected.

Gene lists from the two analysis methods were obtained for each compound. When these analyses were performed, it was observed that there were 123 polynucleotides and polypeptides as listed in Table 3 to be correlated with the drug resistance/sensitivity classification for compound BMS-A or BMS-D as shown in FIG. 1. Of the 123 polynucleotides and polypeptides, 60 were highly expressed in the cell lines that were classified as sensitive to BMS-A or BMS-D, and 63 polynucleotides and polypeptides were highly expressed in the cell lines that were classified as resistant to BMS-A or BMS-D. The same approach was used to select polynucleotides and polypeptides (are listed in Tables 4 and 5) correlated with the drug resistance/sensitivity classification for BMS-B and BMS-C, respectively. The expression patterns of the polynucleotides and polypeptides listed in Tables 3-5 are presented in FIGS. 1-3, which showed correlation with drug resistance/sensitivity classifications for the compound BMS-A/BMS-D, BMS-B and BMS-C, respectively.

Tables 3-5 also show that 73 polynucleotides and polypeptides selected from the above-described analyses are in common among all of the four test compounds (common polynucleotides and polypeptides are shown in Table 6). Thirty-one of the common polynucleotides and polypeptides are highly expressed in cell lines that are classified as sensitive, and 42 of the polynucleotides and polypeptides are highly expressed in cell lines that are classified as resistant. Because these common polynucleotides and polypeptides correlate with drug sensitivity and resistance classifications, they can be used to build predictors for other biological systems as described below.

As used herein, the terms “agent” or “compounds” are meant to encompass any composition capable of modulating a protein tyrosine kinase of the present invention including Src, Fgr, Fyn, Yes, Blk, Hck, Lck and Lyn, as well as other protein tyrosine kinases, including, Bcr-abl, Jak, PDGFR, c-kit and Ephr, either directly or indirectly, and includes small molecule compounds, antisense reagents, antibodies, and the like.

As used herein, the terms “modulate” or “modulates” refer to an increase or decrease in the amount, quality or effect of a particular activity, DNA, RNA, or protein. The definition of “modulate” or “modulates” as used herein is meant to encompass agonists and/or antagonists of a particular activity, DNA, RNA, or protein. The term “modulate” or “modulates” is also meant to encompass an increase or decrease in cellular activity, which necessarily includes a cells ability to differentiate, proliferate, mobilize, metastasize, and/or any other activity that may be associated with a cells transformation into a proliferative and/or oncogenic state.

### Utility of Highly Correlated Polynucleotides and Polypeptides to Make Predictions

Genes that correlate to a specific property of a biological system can be used to make predictions about that biological system and other biological systems. The Genecluster software can be used to select polynucleotides and polypeptides and combinations of polynucleotides and polypeptides that can predict properties using a “weighted-voting cross-validation algorithm” (T. R. Golub et al., 1999, Science, 286:531-537). In particular, the Genecluster software was used to build predictors that demonstrate the utility of polynucleotides and polypeptides that correlate to drug sensitivity and resistance.

As used herein, the terms “predictor” or “predictor sets” are used as follows: a predictor refers to a single gene, or combination of polynucleotides and polypeptides, whose expression pattern or properties can be used to make predictions, with different error rates, about a property or characteristic of any given biological system.

The ability of gene expression patterns to predict a resistance/sensitive classification was further investigated using a Weighted Voting algorithm which uses a cross-validation strategy as described by T. R. Golub et al., 1999, Science, 286:531-537. The program was formatted to select the optimal number of polynucleotides and polypeptides whose expression pattern could be used to predict, with optimal accuracy, the classification of a cell line based on resistance or sensitivity toward a src tyrosine kinase inhibitor compound, e.g., BMS-A, BMS-B, BMS-C or BMS-D. A brief description of the cross-validation strategy of the program is described.

Based on the leave one out cross-validation strategy, a total of thirty-one prediction analyses (i.e., the number of cell lines in the data set) were performed in an iterative manner and the results of all thirty-one prediction analyses were combined to select the optimal number of polynucleotides and polypeptides that had optimal predictive accuracy. In each separate prediction analysis, one cell line was withheld from the data set, and an optimal number gene predictor was built based on the remaining thirty cell lines and subsequently used to predict the class of the withheld sample.

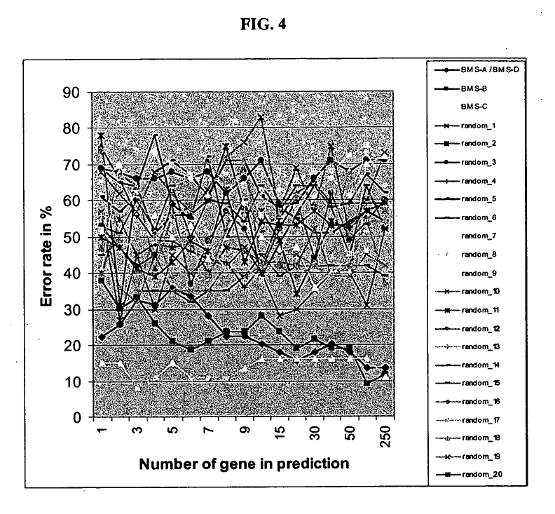

FIG. 4 shows the real error rates using different numbers of polynucleotides and polypeptides in the predictor set for predicting resistant and sensitive classes to BMS-A, BMS-B, BMS-C and BMS-D in the colon cell lines. The real error rates were compared with the real error rates using the same number of polynucleotides and polypeptides as the predictor set in 20 cases, in which classification for the colon cell lines was randomly assigned. For example, when each predictor set contained 20 polynucleotides and polypeptides, the real error rate for BMS-A or BMS-D was 15.7%; for BMS-B and BMS-C, the real error rates were 19% and 16.2%, respectively. This result demonstrated that these error rate values are significantly lower than the real error rates obtained when random phenotype classifications are used for the cell lines (i.e., in a range of from 30% to 70%).

Table 7 presents a true resistance/sensitivity prediction of the 31 colon cell lines for BMS-A or BMS-D, BMS-B and BMS-C using 10 markers as a predictor set (as listed in Table 10). For BMS-A or BMS-D, twenty-eight out of thirty-one cell lines were correctly predicted using the optimal 10-gene predictor set. Two resistant cell lines, CX-1 and SW-403, were predicted to be sensitive to BMS-A or BMS-D, while one sensitive cell lines, HCT-15, were predicted to be resistant to BMS-A or BMS-D. This resulted in a 10%% real error rate (the real error rate is calculated by taking the average of the error rate in each class), calculated as follows:

$$\frac{( 2 / 22 resistant + 1 / 9 sensitive )}{2} \times 100 \% \text{)}$$

Different real error rates were obtained for BMS-B and for BMS-C. For BMS-B, the optimal 10-gene predictor correctly predicted the sensitivity or resistance of 28 cell lines. The predictor made three errors. Two wrong predictions were made in the sensitive classes (calling them resistant). This resulted in an 11.6% real error rate calculated as follows:

$$\frac{( 1 / 20 resistant + 2 / 11 sensitive )}{2} \times 100 \% \text{)}$$

For BMS-C, the optimal 10-gene predictor set predicted 29 cell lines correctly. The predictor only made 2 errors in the sensitive classes. This resulted in an 8.3% real error rate calculated as follows:

$$\frac{( 0 / 19 resistant + 2 / 12 sensitive )}{2} \times 100 \% \text{)}$$

In addition, a confidence score for each prediction made on a cell line by the predictor set can be obtained from the Genecluster software. The confidence score ranges from 0 to 1, measuring the margin of victory in each prediction using weighted-voting algorithms (see T. R. Golub et al., 1999, Science, 286:531-537). The confidence score values for each cell line using the optimal 10-gene predictor set obtained as described are shown in Tables 7.

It will be appreciated that the exact number of polynucleotides and polypeptides that should comprise an optimal predictor set is not definitely established or defined. It is unlikely in the real world that any predictor set can be obtained with 100% accuracy. This is due to the fact that there is a trade-off between the amount of additional information and robustness that are gained by adding more polynucleotides and polypeptides, and the amount of noise that is concomitantly added. In accordance with the present invention, different numbers of polynucleotides and polypeptides were tested in the predictor sets; data were obtained, analyzed and presented for a predictor set comprising 10, or 15 or 25 predictor or marker polynucleotides and polypeptides as demonstrated in Table 7-9. The selection of marker polynucleotides and polypeptides for use in the prediction set was well within the total number of polynucleotides and polypeptides that strongly correlated with the sensitivity class distinction (Tables 3-6). As shown in Table 8, when a predictor set comprising 15 of marker polynucleotides and polypeptides (as listed in Table 11), the error rate for prediction sensitivity of BMS-A/BMS-D, BMS-B and BMS-C was 12.4%, 7% and 4%, respectively. Again, different error rates were obtained when a predictor set comprising 25 of marker polynucleotides and polypeptides as shown in Table 9 and Table 12.

Thus, in accordance with the present invention, an approach has been developed in which polynucleotides and polypeptides and combinations of polynucleotides and polypeptides have been discovered, whose expression pattern in a subset of cell lines correlates with, and can be used as a predictor of, in vitro response to treatment with a series of compounds that inhibit the function of src tyrosine kinases.

### Predictor Sets, Error Rates and Algorithms used to Demonstrate Utility

The number of polynucleotides and polypeptides in any given predictor may influence the error rate of the predictor set in cross-validation experiments and with other mathematical algorithms. The data show that the error rate of a predictor is somewhat dependent on the number of polynucleotides and polypeptides in the predictor set and the contribution of each individual gene in the given predictor set and the number of cell lines that are tested in the cross validation experiment. For example, in a given predictor set, one gene may contribute more significantly than the other polynucleotides and polypeptides to the prediction.

It is very likely that if a gene significantly contributes to a predictor set, then it can be used in different combinations with other polynucleotides and polypeptides to achieve different error rates in different predictor sets, e.g., gene A alone gives an error rate of 30%. In combination with polynucleotides and polypeptides, B, C and D, the error rate becomes 10%; in combination with polynucleotides and polypeptides B, D and E, the error rate becomes 12%; while a combination of gene A with polynucleotides and polypeptides E-X gives an error rate of 8%, and so on. The error rates as described herein apply to the set of cell lines used in the cross-validation experiment. If a different set is used, or more cell lines are added to the original set tested, then different error rates may be obtained as described and understood by the skilled practitioner. Importantly, different combinations of polynucleotides and polypeptides that correlate to drug sensitivity can be used to build predictors with different prediction accuracy.

### Applications of Predictor Sets

Predictor sets with different error rates may be used in different applications. Predictor sets can be built from any combination of the polynucleotides and polypeptides listed in Tables 3-6, or the predictor gene subsets of 25, 15, and 7 polynucleotides and polypeptides, as presented in Tables 7, 8, 9, 10, 11, and 12, respectively, to make predictions about the likely effect of either src tyrosine inhibitor compounds or compounds that affect the src tyrosine kinase signaling pathway in different biological systems. The various predictor sets described herein, or the combination of these predictor sets with other polynucleotides and polypeptides or other co-variants of these polynucleotides and polypeptides, are likely to have broad utility. For example, they can be used as diagnostic or prognostic indicators in disease management; they can be used to predict how patients with cancer might respond to therapeutic intervention with compounds that modulate the src tyrosine kinase family; and they can be used to predict how patients might respond to therapeutic intervention that modulate signaling through the entire src tyrosine kinase regulatory pathway.

While the data described herein were generated in cell lines that are routinely used to screen and identify compounds that have potential utility for cancer therapy, the predictors may have both diagnostic and prognostic value in other diseases areas in which signaling through protein tyrosine kinases, particularly src tyrosine kinase or the src tyrosine kinase pathway is of importance, e.g., in immunology, or in cancers or tumors in which cell signaling and/or proliferation controls have gone awry. Such protein tyrosine kinases and their pathways comprise, for example, members of the Src family of tyrosine kinases, for example, Src, Fgr, Fyn, Yes, Blk, Hck, Lck and Lyn, as well as other protein tyrosine kinases, including, Bcr-abl, Jak, PDGFR, c-kit and Ephr.

Further, although the data described herein have been generated using the particularly exemplified src tyrosine kinase inhibitor compounds, namely, BMS-A, BMS-B, BMS-C and BMS-D, the predictors may have both diagnostic and prognostic value related to any molecules or therapeutic interventions that affect src tyrosine kinases or the src tyrosine kinase signaling pathways.

Those having skill in the pertinent art will appreciate that protein tyrosine kinase pathways, e.g., the Src tyrosine kinase pathway, is used and functional in cell types other than cell lines of colon tissue. Therefore, the described predictor set of polynucleotides and polypeptides, or combinations of polynucleotides and polypeptides within the predictor set, may show utility for predicting drug sensitivity or resistance to compounds that interact with or inhibit the src tyrosine kinase activity in cells from other tissues or organs associated with a disease state, or cancers or tumors derived from other tissue types. Non-limiting examples of such cells, tissues and organs include colon, breast, lung, prostate, testes, ovaries, cervix, esophagus, pancreas, spleen, liver, kidney, stomach, lymphocytic and brain, thereby providing a broad and advantageous applicability to the predictor gene sets described herein. Cells for analysis can be obtained by conventional procedures as known in the art, for example, tissue biopsy, aspiration, sloughed cells, e.g., colonocytes, clinical or medical tissue or cell sampling procedures.

### Functionality of Polynucleotides and Polypeptides that Make Up a Predictor Set

The use of a predictor, or predictor set, (e.g., predictor polynucleotides and polypeptides, or a predictor set of polynucleotides and polypeptides) is simply for predicting an outcome prior to having any knowledge about a biological system. Essentially, a predictor can be considered to be a statistical tool. The predictor is useful primarily in predicting the phenotype that is used to classify the biological system. In the specific embodiment provided by the present invention, the classification as “resistant” or “sensitive” is based on the log₁₀(IC₅₀) value of each cell line to a compound (e.g., the src kinase inhibitor compounds BMS-A, BMS-B, BMS-C or BMS-D as exemplified herein), relative to the mean log₁₀(IC₅₀) value of the cell line panel (e.g., a thirty-one colon cell line panel, as exemplified herein).

A number of the polynucleotides and polypeptides as described herein (Tables 3-6) are known to be substrates for the src tyrosine kinase family, e.g., caveolin-1, caveolin-2, phosphoinositide 3-kinase, etc., (M. T. Brown and J. A. Cooper, 1996, Biochemica et Biophysica Acta, 1287:121-149). This is expected, since polynucleotides and polypeptides that contribute to a high predictor accuracy are likely to play a functional role in the pathway that is being modulated. For example, Herceptin therapy (i.e., antibody that binds to the Her2 receptor and prevents function via internalization) is indicated when the Her2 gene is overexpressed. It is unlikely that a therapy will have any therapeutic effect if the target enzyme is not expressed.

However, although the complete function of all of the polynucleotides and polypeptides and their functional products (proteins and mRNAs) that make up a predictor set are not currently known, some of the polynucleotides and polypeptides are likely to be directly involved in the src tyrosine signaling pathway. In addition, some of the polynucleotides and polypeptides in the predictor set may be indirectly related to src signaling pathways. In addition, some of the polynucleotides and polypeptides in the predictor set may function in the metabolic or other resistance pathways specific to the compounds tested. Notwithstanding, a knowledge about the function of the polynucleotides and polypeptides is not a requisite for determining the accuracy of a predictor according to the practice of the present invention.

It has been demonstrated that different predictor sets are necessary to achieve the lowest error rate for the different compounds as tested herein. This is due to the subset of the cell lines that show different responses to the different compounds. Therefore, in the discovery process of building a predictor, the classification of a cell as either resistant or sensitive to a particular compound, or series of compounds, will impact the final set of polynucleotides and polypeptides that comprise the best predictor/predictor set. Because different combinations of resistant and sensitive cells were used for each compound, different predictor sets were obtained. In addition, obtaining different predictor sets for different compounds can be avoided if those cell lines having common resistant or sensitive classifications of gene marker expression are use (see, e.g., the 26 cell lines presented in Table 2).

The data presented herein also reveal that there are common polynucleotides and polypeptides for the four different compounds (see, e.g., Table 6). It is likely that these polynucleotides and polypeptides will have some role, whether direct or indirect, in the src tyrosine kinase pathway. Alternatively, these polynucleotides and polypeptides can be important in intrinsically determining the sensitivity of a cell to src signaling or inhibition.

As described herein, polynucleotides and polypeptides have been discovered that correlate to the relative intrinsic sensitivity or resistance of colon cell lines to treatment with compounds that interact with and inhibit src tyrosine kinases. These polynucleotides and polypeptides have been shown, through a weighted voting cross validation program, to have utility in predicting the intrinsic resistance and sensitivity of colon cell lines to these compounds.

An embodiment of the present invention relates to a method of determining or predicting if an individual requiring drug or chemotherapeutic treatment or therapy for a disease, for example, a cancer or tumor of a particular type, will be likely to successfully respond or not respond to the drug or chemotherapeutic agent prior to subjecting the individual to such treatment or chemotherapy. Preferably, the drug or chemotherapeutic agent is one that modulates protein tyrosine kinases, particularly src activity or src family tyrosine kinases activity or signaling involving src or src family tyrosine kinases. In accordance with the method of the invention, cells from a tissue or organ associated with disease, e.g., a patient biopsy of a tumor or cancer, preferably a colon cancer or tumor biopsy, are subjected to an in vitro assay as described herein, to determine their marker gene expression pattern (polynucleotides and polypeptides from Table 3-6) prior to their treatment with the compound or drug, particularly a protein tyrosine kinase inhibitor, preferably a src kinase inhibitor. The resulting gene expression profile of the cells before drug treatment is compared with the gene expression pattern of the same polynucleotides and polypeptides in cells that are either resistant or sensitive to the drug or compound, as provided by the present invention, i.e., FIGS. 1-3.

Success or failure of treatment of a patient's cancer or tumor with the drug can be determined based on the gene expression pattern of the patient's cells being tested, compared with the gene expression pattern of the predictor polynucleotides and polypeptides in the resistant or sensitive panel of that have been exposed to the drug or compound and subjected to the predictor gene analysis detailed herein. Thus, if, following exposure to the drug, the test cells show a gene expression pattern corresponding to that of the predictor gene set of the control panel of cells that are sensitive to the drug or compound, it is highly likely or predicted that the individual's cancer or tumor will respond favorably to treatment with the drug or compound. By contrast, if, after drug exposure, the test cells show a gene expression pattern corresponding to that of the predictor gene set of the control panel of cells that are resistant to the drug or compound, it is highly likely or predicted that the individual's cancer or tumor will not respond to treatment with the drug or compound.

As a related and more particular embodiment, the present invention relates to a method of determining or predicting if an individual requiring drug or chemotherapeutic treatment or therapy for a disease, for example, a breast cancer or a breast tumor, will be likely to successfully respond or not respond to the drug or chemotherapeutic agent prior to subjecting the individual to such treatment or chemotherapy. In this embodiment, the drug or chemotherapeutic agent is preferably one that modulates src tyrosine kinase activity or signaling involving src tyrosine kinase. In accordance with the method of the invention, cells from a tissue or organ associated with disease, e.g., a patient biopsy of a tumor or cancer, preferably a colon cancer or tumor biopsy, are subjected to an in vitro assay as described herein, to determine their marker gene expression pattern (polynucleotides and polypeptides from Tables 3 thru 6 and/or the predictor gene subsets of Tables 10 thru 12) prior to their treatment with the src tyrosine kinase inhibitor compound or drug. The resulting gene expression profile of the cells before drug treatment is compared with the gene expression pattern of the same polynucleotides and polypeptides in cells that are either resistant or sensitive to the drug or compound, as provided by the present invention.

In another related embodiment, the present invention includes a method of predicting, prognosing, diagnosing, and/or determining whether an individual requiring drug therapy for a disease state or chemotherapeutic for cancer (e.g., colon cancer) will or will not respond to treatment prior to administration of treatment. The treatment or therapy preferably involves a protein tyrosine kinase modulating agent, compound, or drug, for example, an inhibitor of the protein tyrosine kinase activity. Protein tyrosine kinases include, without limitation, members of the Src family of tyrosine kinases, for example, Src, Fgr, Fyn, Yes, Blk, Hck, Lck and Lyn, as well as other protein tyrosine kinases, including, Bcr-abl, Jak, PDGFR, c-kit and Ephr. Preferred is src tyrosine kinase and inhibitors thereof. In accordance with this embodiment, cells from a patient's tissue sample, e.g., a colon tumor or cancer biopsy, are assayed to determine their gene expression pattern prior to treatment with the protein tyrosine kinase modulating agent, compound, or drug. The resulting gene expression profile of the test cells before exposure to the compound or drug is compared with that of one or more of the predictor subsets of polynucleotides and polypeptides comprising either 25, 15, or 10 polynucleotides and polypeptides as described herein and shown in Tables 10 thru 12, respectively.

In a related embodiment, screening assays are provided for determining if a patient's cancer or tumor is or will be susceptible or resistant to treatment with a drug or compound, particularly, a drug or compound directly or indirectly involved in src or src family tyrosine kinases activity or the src kinase pathway.

Also provided are monitoring assays to monitor the progress of a drug treatment involving drugs or compounds that interact with or inhibit protein tyrosine kinases, particularly src or src family tyrosine kinases activity. Protein tyrosine kinases encompassed by these monitoring assays include members of the Src family of tyrosine kinases, for example, Src, Fgr, Fyn, Yes, Blk, Hck, Lck and Lyn, as well as other protein tyrosine kinases, including, Bcr-abl, Jak, PDGFR, c-kit and Ephr. Such in vitro assays are capable of monitoring the treatment of a patient having a disease treatable by a compound or agent that modulates or interacts with a src tyrosine kinase by comparing the resistance or sensitivity gene expression pattern of cells from a patient tissue sample, e.g., a tumor or cancer biopsy, preferably a colon cancer or tumor sample, prior to treatment with a drug or compound that inhibits src or src family tyrosine kinases activity and again following treatment with the drug or compound with the expression pattern of one or more of the predictor gene sets described, or combinations thereof. Isolated cells from the patient are assayed to determine their gene expression pattern before and after exposure to a compound or drug, preferably a src or src family tyrosine kinases inhibitor, to determine if a change of the gene expression profile has occurred so as to warrant treatment with another drug or agent, or discontinuing current treatment. The resulting gene expression profile of the cells tested before and after treatment is compared with the gene expression pattern of the predictor set of polynucleotides and polypeptides that have been described and shown herein to be highly expressed in cells that are either resistant or sensitive to the drug or compound. Alternatively, a patient's progress related to drug treatment or therapy can be monitored by obtaining a gene expression profile as described above, only after the patient has undergone treatment with a given drug or therapeutic compound. In this way, there is no need to test a patient sample prior to treatment with the drug or compound.

Such a monitoring process can indicate success or failure of a patient's treatment with a drug or compound based on the gene expression pattern of the cells isolated from the patient's sample, e.g., a tumor or cancer biopsy, as being relatively the same as or different from the gene expression pattern of the predictor gene set of the resistant or sensitive control panel of cells that have been exposed to the drug or compound and assessed for their gene expression profile following exposure. Thus, if, after treatment with a drug or compound, the test cells show a change in their gene expression profile from that seen prior to treatment to one which corresponds to that of the predictor gene set of the control panel of cells that are resistant to the drug or compound, it can serve as an indicator that the current treatment should be modified, changed, or even discontinued. Also, should a patient's response be one that shows sensitivity to treatment by a src or src family tyrosine kinases inhibitor compound, based on correlation of the expression profile of the predictor polynucleotides and polypeptides of cells showing drug sensitivity with the gene expression profile from cells from a patient undergoing treatment, the patient's treatment prognosis can be qualified as favorable and treatment can continue. Further, if a patient has not been tested prior to drug treatment, the results obtained after treatment can be used to determine the resistance or sensitivity of the cells to the drug based on the gene expression profile compared with the predictor gene set.

In a related embodiment, the present invention embraces a method of monitoring the treatment of a patient having a disease treatable by a compound or agent that modulates a protein tyrosine kinase, i.e., colon cancer. Protein tyrosine kinases encompassed by such treatment monitoring assays include members of the Src family of tyrosine kinases, for example, Src, Fgr, Fyn, Yes, Blk, Hck, Lck and Lyn, as well as other protein tyrosine kinases, including, Bcr-abl, Jak, PDGFR, c-kit and Ephr. For these assays, test cells from the patient are assayed to determine their gene expression pattern before and after exposure to a protein tyrosine kinase inhibitor compound or drug. The resulting gene expression profile of the cells tested before and after treatment is compared with the gene expression pattern of the predictor set of polynucleotides and polypeptides that have been described and shown herein to be highly expressed in cells that are either resistant or sensitive to the drug or compound. Thus, if a patient's response is or becomes one that is sensitive to treatment by a protein tyrosine kinase inhibitor compound, based on correlation of the expression profile of the predictor polynucleotides and polypeptides, the patient's treatment prognosis can be qualified as favorable and treatment can continue. Also, if after treatment with a drug or compound, the test cells do not exhibit a change in their gene expression profile to a profile that corresponds to that of the control panel of cells that are sensitive to the drug or compound, this serves as an indicator that the current treatment should be modified, changed, or even discontinued. Such monitoring processes can be repeated as necessary or desired and can indicate success or failure of a patient's treatment with a drug or compound, based on the gene expression pattern of the cells isolated from the patient's sample. The monitoring of a patient's response to a given drug treatment can also involve testing the patient's cells in the assay as described, only after treatment, rather than before and after treatment, with drug or active compound.

In a preferred embodiment, the present invention embraces a method of monitoring the treatment of a patient having a disease treatable by a compound or agent that modulates a src tyrosine kinase, i.e., colon cancer. The test cells from the patient are assayed to determine their gene expression pattern before and after exposure to a src tyrosine kinase inhibitor compound or drug. The resulting gene expression profile of the cells tested before and after treatment is compared with the gene expression pattern of the predictor set of polynucleotides and polypeptides that have been described and shown herein to be highly expressed in cells that are either resistant or sensitive to the drug or compound. Thus, if a patient's response is or becomes one that is sensitive to treatment by a src tyrosine kinase inhibitor compound, based on correlation of the expression profile of the predictor polynucleotides and polypeptides, the patient's treatment prognosis can be qualified as favorable and treatment can continue. Also, if after treatment with a drug or compound, the test cells do not exhibit a change in their gene expression profile to a profile that corresponds to that of the control panel of cells that are sensitive to the drug or compound, this serves as an indicator that the current treatment should be modified, changed, or even discontinued. Such monitoring processes can be repeated as necessary or desired and can indicate success or failure of a patient's treatment with a drug or compound, based on the gene expression pattern of the cells isolated from the patient's sample. The monitoring of a patient's response to a given drug treatment can also involve testing the patient's cells in the assay as described only after treatment, rather than before and after treatment, with drug or active compound.

In another embodiment, the present invention encompasses a method of classifying whether a biological system, preferably cells from a tissue, organ, tumor or cancer of an afflicted individual, will be resistant or sensitive to a compound that modulates the system. In a preferred aspect of this invention, the sensitivity or resistance of cells, e.g., those obtained from a tumor or cancer, to a src tyrosine kinase inhibitor compound, or series of compounds, is determined. According to the method, a resistance/sensitivity profile of the cells after exposure to the src kinase inhibitor drug or compound can be determined via gene expression profiling protocols set forth herein. Such resistance/sensitivity profile of the cells reflects an IC₅₀ value of the cells to the compound(s) as determined using a suitable assay, such as an in vitro cytotoxicity assay as described in Example 1. A procedure of this sort can be performed using a variety of cell types and compounds that interact with src tyrosine kinase, or affect its activity in the src or src family tyrosine kinases signaling pathway.

In another of its embodiments, the present invention contemplates the preparation of one or more specialized microarrays (e.g., oligonucleotide microarrays or cDNA microarrays) comprising all of the polynucleotides and polypeptides in the Tables 3-5, or combinations thereof, of the predictor gene sets described herein that have been demonstrated to be most highly correlated with sensitivity (or resistance) to src or src family tyrosine kinases modulators, particularly inhibitors of src tyrosine kinase. Preferably, the predictor gene sets are common for predicting sensitivity among more than one src kinase modulator, e.g. a src kinase inhibitor, as demonstrated herein. In accordance with this aspect of the invention, the oligonucleotide sequences or cDNA sequences include any of the predictor polynucleotides and polypeptides or gene combinations as described herein, which are highly expressed in resistant or sensitive cells, and are contained on a microarray, e.g., a oligonucleotide microarray or cDNA microarray in association with, or introduced onto, any supporting materials, such as glass slides, nylon membrane filters, glass or polymer beads, or other types of suitable substrate material.

Cellular nucleic acid, e.g., RNA, is isolated either from cells undergoing testing after exposure to a drug or compound that interacts with src tyrosine kinase, or its signaling pathway, or from cells being tested to obtain an initial determination or prediction of cells' sensitivity to the drug or compound, and, ultimately, a prediction of treatment outcome with the drug or compound. The isolated nucleic acid is appropriately labeled and applied to one or more of the specialized microarrays. The resulting pattern of gene expression on the specialized microarray is analyzed as described herein and known in the art. A pattern of gene expression correlating with either sensitivity or resistance to the drug or compound is able to be determined, e.g., via comparison with the gene expression patterns as shown in FIGS. 1-3 for the panel of cells exposed to the src kinase inhibitors assayed herein.

In accordance with the specialized microarray embodiment of this invention, the microarray contains the polynucleotides and polypeptides of one or more of the predictor gene sets, or a combination thereof, or all of the gene in the Tables 3-5, that are highly correlated with drug sensitivity or resistance by a cell type. (See, for example, Table 1 for colon cells). If the nucleic acid target isolated from test cells, such as tumor or cancer cells, preferably colon cancer or tumor cells, shows a high level of detectable binding to the polynucleotides and polypeptides of the predictor set for drug sensitivity relative to control, then it can be predicted that a patient's cells will respond to the drug, or a series of drugs, and that the patient's response to the drug, or a series of drugs, will be favorable.

Such a result predicts that the cells of a tumor or cancer are good candidates for the successful treatment or therapy utilizing the drug, or series of drugs. Alternatively, if the nucleic acid target isolated from test cells shows a high level of detectable binding to the polynucleotides and polypeptides of the predictor set for drug resistance, relative to control, then it can be predicted that a patient is likely not to respond to the drug, or a series of drugs, and that the patient's response to the drug, or a series of drugs, is not likely to be favorable. Such a result predicts that the cells of a tumor or cancer are not good candidates for treatment or therapy utilizing the drug, or series of drugs.

The utilization of microarray technology is known practiced in the art. Briefly, to determine gene expression using microarray technology, polynucleotides, e.g., RNA, DNA, cDNA, preferably RNA, are isolated from a biological sample, e.g., cells, as described herein for colon cells. The isolated nucleic acid is detectably labeled, e.g., fluorescent, enzyme, or chemiluminescent label, and applied to a microarray, e.g., the specialized microarrays provided by this invention. The array is then washed to remove unbound material and visualized by staining or fluorescence, or other means known in the art depending on the type of label utilized.

In another embodiment of this invention, the predictor gene sets, or subsets of polynucleotides and polypeptides comprising the predictor gene sets, can be used as biomarkers for cells that are resistant or sensitive to src kinase inhibitor compounds. With the predictor polynucleotides and polypeptides in hand, screening and detection assays can be carried out to determine whether or not a given compound, preferably a src kinase inhibitor compound, elicits a sensitive or a resistant phenotype following exposure of cells, e.g., a tumor or cancer biopsy sample, such as a colon cancer cell sample, to the compound. Thus, methods of screening, monitoring, detecting, and/or diagnosing to determine the resistance or sensitivity of cells to a drug or compound that interacts with src tyrosine kinase, or the src kinase pathway, preferably an inhibitor compound, and to which the cells are exposed, are encompassed by the present invention.

Such methods embrace a variety of methods and assays to determine and assess the expression of polynucleotides and polypeptides, in particular, the predictor or src biomarker polynucleotides and polypeptides as described herein (Tables 3-6), in cells that have been exposed to drugs or compounds that interact with or effect a protein tyrosine kinase, or a protein tyrosine kinase pathway, wherein the protein tyrosine kinases include members of the Src family of tyrosine kinases, for example, Src, Fgr, Fyn, Yes, Blk, Hck, Lck and Lyn, as well as other protein tyrosine kinases, including, Bcr-abl, Jak, PDGFR, c-kit and Ephr. Suitable methods include detection and evaluation of gene activation or expression at the level of nucleic acid, e.g., DNA, RNA, MRNA, and detection and evaluation of encoded protein. For example, PCR assays as known and practiced in the art can be employed to quantify RNA in cells being assayed for susceptibility to drug treatment, for example, src kinase inhibitors. (see Example 2, RT-PCR).

In another embodiment, the present invention is directed to a method of identifying cells, tissues, and/or patients that are predicted to be resistant to either protein tyrosine inhibitor compounds or compounds that affect protein tyrosine kinase signaling pathways, e.g., Src tyrosine kinase, or that are resistant in different biological systems to those compounds. The method comprises the step(s) of (i) analyzing the expression of only those polynucleotides and polypeptides listed in Tables 3 thru 6, or any combination thereof, that have been shown to be correlative to predicting resistant responses to such compounds; (ii) comparing the observed expression levels of those correlative resistant polynucleotides and polypeptides in the test cells, tissues, and/or patients to the expression levels of those same polynucleotides and polypeptides in a cell line that is known to be resistant to the compounds; and (iii) predicting whether the cells, tissues, and/or patients are resistant to the compounds based upon the overall similarity of the observed expression of those polynucleotides and polypeptides in step (ii).

In another embodiment, the present invention is directed to a method of identifying cells, tissues, and/or patients that are predicted to be sensitive to either protein tyrosine inhibitor compounds or compounds that affect protein tyrosine kinase signaling pathways, e.g., the Src tyrosine kinase, or that are sensitive in different biological systems to those compounds. The method involves the step(s) of (i) analyzing the expression of only those polynucleotides and polypeptides listed in Tables 3 thru 6, or any combination thereof, that have been shown to be correlative to predicting sensitive responses to such compounds; (ii) comparing the observed expression levels of those correlative sensitive polynucleotides and polypeptides in the test cells, tissues, and/or patients to the expression levels of those same polynucleotides and polypeptides in a cell line that is known to be sensitive to the compounds; and (iii) predicting whether the cells, tissues, and/or patients are sensitive to the compounds based upon the overall similarity of the observed expression of those polynucleotides and polypeptides in step (ii).

The present invention further encompasses the detection and/or quantification of one or more of the protein tyrosine kinase biomarker proteins of the present invention using antibody-based assays (e.g., immunoassays) and/or detection systems. As mentioned herein, protein tyrosine kinase biomarkers encompass members of the Src family of tyrosine kinases, for example, Src, Fgr, Fyn, Yes, Blk, Hck, Lck and Lyn, as well as other protein tyrosine kinases, including, Bcr-abl, Jak, PDGFR, c-kit and Ephr. Such assays include the following non-limiting examples, ELISA, immunofluorescence, FACS, Western Blots, etc., as further described herein.

In another embodiment, the human protein tyrosine kinase biomarker polypeptides and/or peptides of the present invention, or immunogenic fragments or oligopeptides thereof, can be used for screening therapeutic drugs or compounds in a variety of drug screening techniques. The fragment employed in such a screening assay can be free in solution, affixed to a solid support, borne on a cell surface, or located intracellularly. The reduction or abolition of activity of the formation of binding complexes between the biomarker protein and the agent being tested can be measured. Thus, the present invention provides a method for screening or assessing a plurality of compounds for their specific binding affinity with a protein kinase biomarker polypeptide, or a bindable peptide fragment thereof, of this invention. The method comprises the steps of providing a plurality of compounds; combining the protein kinase biomarker polypeptide, or a bindable peptide fragment thereof, with each of the plurality of compounds, for a time sufficient to allow binding under suitable conditions; and detecting binding of the biomarker polypeptide or peptide to each of the plurality of test compounds, thereby identifying the compounds that specifically bind to the biomarker polypeptide or peptide. More specifically, the biomarker polypeptide or peptide is that of a Src tyrosine kinase.

Methods of identifying compounds that modulate the activity of the human protein tyrosine kinase biomarker polypeptides and/or peptides are provided by the present invention and comprise combining a potential or candidate compound or drug modulator of protein kinase biological activity with an protein kinase biomarker polypeptide or peptide, for example, the Src tyrosine kinase biomarker amino acid sequences as set forth in Table 2, and measuring an effect of the candidate compound or drug modulator on the biological activity of the protein kinase biomarker polypeptide or peptide. Such measurable effects include, for example, a physical binding interaction; the ability to cleave a suitable protein kinase substrate; effects on a native and cloned protein kinase biomarker-expressing cell line; and effects of modulators or other protein kinase-mediated physiological measures.

Another method of identifying compounds that modulate the biological activity of the novel protein tyrosine kinase biomarker polypeptides of the present invention comprises combining a potential or candidate compound or drug modulator of a protein tyrosine kinase biological activity, e.g., a Src tyrosine kinase, with a host cell that expresses the protein tyrosine kinase biomarker polypeptide and measuring an effect of the candidate compound or drug modulator on the biological activity of the protein tyrosine kinase biomarker polypeptide. The host cell can also be capable of being induced to express the protein tyrosine kinase biomarker polypeptide, e.g., via inducible expression. Physiological effects of a given modulator candidate on the protein tyrosine kinase biomarker polypeptide can also be measured. Thus, cellular assays for particular protein tyrosine kinase modulators, e.g., a src kinase modulator, can be either direct measurement or quantification of the physical biological activity of the protein tyrosine kinase biomarker polypeptide, or they may be measurement or quantification of a physiological effect. Such methods preferably employ a protein tyrosine kinase biomarker polypeptide as described herein, or an overexpressed recombinant protein tyrosine kinase biomarker polypeptide in suitable host cells containing an expression vector as described herein, wherein the protein tyrosine kinase biomarker polypeptide is expressed, overexpressed, or undergoes up-regulated expression.

Another aspect of the present invention embraces a method of screening for a compound that is capable of modulating the biological activity of a protein tyrosine kinase biomarker polypeptide, e.g., a Src kinase biomarker polypeptide. The method comprises providing a host cell containing an expression vector harboring a nucleic acid sequence encoding a protein tyrosine kinase biomarker polypeptide, or a functional peptide or portion thereof (e.g., the src polypeptide, protein, peptide, or fragment sequences as set forth in Tables 3 thru 12, or the Sequence Listing herein); determining the biological activity of the expressed protein tyrosine kinase biomarker polypeptide in the absence of a modulator compound; contacting the cell with the modulator compound and determining the biological activity of the expressed protein tyrosine kinase biomarker polypeptide in the presence of the modulator compound. In such a method, a difference between the activity of the protein tyrosine kinase biomarker polypeptide in the presence of the modulator compound and in the absence of the modulator compound indicates a modulating effect of the compound.

Essentially any chemical compound can be employed as a potential modulator or ligand in the assays according to the present invention. Compounds tested as protein tyrosine kinase modulators can be any small chemical compound, or biological entity (e.g., protein, sugar, nucleic acid, or lipid). Test compounds are typically small chemical molecules and peptides. Generally, the compounds used as potential modulators can be dissolved in aqueous or organic (e.g., DMSO-based) solutions. The assays are designed to screen large chemical libraries by automating the assay steps and providing compounds from any convenient source. Assays are typically run in parallel, for example, in microtiter formats on microtiter plates in robotic assays. There are many suppliers of chemical compounds, including, for example, Sigma (St. Louis, Mo.), Aldrich (St. Louis, Mo.), Sigma-Aldrich (St. Louis, Mo.), Fluka Chemika-Biochemica Analytika (Buchs, Switzerland). Also, compounds can be synthesized by methods known in the art.

High throughput screening methodologies are particularly envisioned for the detection of modulators of the novel protein tyrosine kinase biomarker, e.g., src biomarker, polynucleotides and polypeptides described herein. Such high throughput screening methods typically. involve providing a combinatorial chemical or peptide library containing a large number of potential therapeutic compounds (e.g., ligand or modulator compounds). The combinatorial chemical libraries or ligand libraries are then screened in one or more assays to identify those library members (e.g., particular chemical species or subclasses) that display a desired characteristic activity. The compounds so identified can serve as conventional lead compounds, or can themselves be used as potential or actual therapeutics.

A combinatorial chemical library is a collection of diverse chemical compounds generated either by chemical synthesis or biological synthesis, prepared by combining a number of chemical building blocks (i.e., reagents such as amino acids). As an example, a linear combinatorial library, e.g., a polypeptide or peptide library, is formed by combining a set of chemical building blocks in every possible way for a given compound length (i.e., the number of amino acids in a polypeptide or peptide compound). Millions of chemical compounds can be synthesized through such combinatorial mixing of chemical building blocks.

The preparation and screening of combinatorial chemical libraries is well known to those having skill in the pertinent art. Combinatorial libraries include, without limitation, peptide libraries (e.g. U.S. Pat. No. 5,010,175; Furka, 1991, Int. J. Pept. Prot. Res., 37:487-493; and Houghton et al., 1991, Nature, 354:84-88). Other chemistries for generating chemical diversity libraries can also be used. Nonlimiting examples of chemical diversity library chemistries include, peptoids (PCT Publication No. WO 91/019735), encoded peptides (PCT Publication No. WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., 1993, Proc. Natl. Acad. Sci. USA, 90:6909-6913), vinylogous polypeptides (Hagihara et al., 1992, J. Amer. Chem. Soc., 114:6568), nonpeptidal peptidomimetics with glucose scaffolding (Hirschmann et al., 1992, J. Amer. Chem. Soc., 114:9217-9218), analogous organic synthesis of small compound libraries (Chen et al., 1994, J. Amer. Chem. Soc., 116:2661), oligocarbamates (Cho et al., 1993, Science, 261:1303), and/or peptidyl phosphonates (Campbell et al., 1994, J. Org. Chem., 59:658), nucleic acid libraries (see Ausubel, Berger and Sambrook, all supra), peptide nucleic acid libraries (U.S. Pat. No. 5,539,083), antibody libraries (e.g., Vaughn et al., 1996, Nature Biotechnology, 14(3):309-314) and PCT/US96/10287), carbohydrate libraries (e.g., Liang et al., 1996, Science, 274-1520-1522) and U.S. Pat. No. 5,593,853), small organic molecule libraries (e.g., benzodiazepines, Baum C&EN, Jan. 18, 1993, page 33; and U.S. Pat. No. 5,288,514; isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); and the like.

Devices for the preparation of combinatorial libraries are commercially available (e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, a large number of combinatorial libraries are commercially available (e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., and the like).

In one aspect, the invention provides solid phase-based in vitro assays in a high throughput format, where the cell or tissue expressing a tyrosine kinase protein/polypeptide/peptide is attached to a solid phase substrate. In such high throughput assays, it is possible to screen up to several thousand different modulators or ligands in a single day. In particular, each well of a microtiter plate can be used to perform a separate assay against a selected potential modulator, or, if concentration or incubation time effects are to be observed, every 5-10 wells can be used to test a single modulator. Thus, a single standard microtiter plate can be used in to assay about 96 modulators. If 1536 well plates are used, then a single plate can easily assay from about 100 to about 1500 different compounds. It is possible to assay several different plates per day; thus, for example, assay screens for up to about 6,000-20,000 different compounds are possible using the described integrated systems.

In another of its aspects, the present invention encompasses screening and small molecule (e.g., drug) detection assays which involve the detection or identification of small molecules that can bind to a given protein, i.e., a tyrosine kinase biomarker polypeptide or peptide, such as a Src tyrosine kinase biomarker polypeptide or peptide. Particularly preferred are assays suitable for high throughput screening methodologies.

In such binding-based detection, identification, or screening assays, a functional assay is not typically required. All that is needed, in general, is a target protein, preferably substantially purified, and a library or panel of compounds (e.g., ligands, drugs, or small molecules), or biological entities to be screened or assayed for binding to the protein target. Preferably, most small molecules that bind to the target protein modulate the target's activity in some manner due to preferential, higher affinity binding to functional areas or sites on the protein.

An example of such an assay is the fluorescence based thermal shift assay (3-Dimensional Pharmaceuticals, Inc., 3DP, Exton, Pa.) as described in U.S. Pat. Nos. 6,020,141 and 6,036,920 to Pantoliano et al. (See also, J. Zimmerman, 2000, Gen. Eng. News, 20(8)). The assay allows the detection of small molecules (e.g., drugs, ligands) that bind to expressed, and preferably purified, tyrosine kinase biomarker proteins/polypeptides/peptides, such as the Src tyrosine kinase, based on affinity of binding determinations by analyzing thermal unfolding curves of protein-drug or ligand complexes. The drugs or binding molecules determined by this technique can be further assayed, if desired, by methods such as those described herein to determine if the molecules affect or modulate function or activity of the target protein.

To purify a tyrosine kinase biomarker polypeptide or peptide, e.g., Src tyrosine kinase, to measure a biological binding or ligand binding activity, the source may be a whole cell lysate that can be prepared by successive freeze-thaw cycles (e.g., one to three) in the presence of standard protease inhibitors. The tyrosine kinase biomarker polypeptide can be partially or completely purified by standard protein purification methods, e.g., affinity chromatography using specific antibody(ies) described herein, or by ligands specific for an epitope tag engineered into the recombinant tyrosine kinase biomarker polypeptide molecule, also as described herein. Binding activity can then be measured as described.

Compounds which are identified according to the methods provided herein, and which modulate or regulate the biological activity or physiology of the tyrosine kinase biomarker polypeptides according to the present invention, are a preferred embodiment of this invention. It is contemplated that such modulatory compounds can be employed in treatment and therapeutic methods for treating a condition that is mediated by the tyrosine kinase biomarker polypeptides, e.g., Src tyrosine kinase biomarker polypeptides, by administering to an individual in need of such treatment a therapeutically effective amount of the compound identified by the methods described herein.

In addition, the present invention provides methods for treating an individual in need of such treatment for a disease, disorder, or condition that is mediated by the tyrosine kinase biomarker polypeptides of the invention, comprising administering to the individual a therapeutically effective amount of the tyrosine kinase biomarker-modulating compound identified by a method provided herein. In accordance with this invention, the tyrosine kinase biomarker polypeptides or proteins encompassed by the methods include members of the Src family of tyrosine kinases, for example, Src, Fgr, Fyn, Yes, Blk, Hck, Lck and Lyn, as well as other protein tyrosine kinases, including, Bcr-abl, Jak, PDGFR, c-kit and Ephr.

The present invention particularly provides methods for treating an individual in need of such treatment for a disease, disorder, or condition that is mediated by Src biomarker polypeptides of the invention, comprising administering to the individual a therapeutically effective amount of the Src biomarker-modulating compound identified by a method provided herein.

Antibodies directed against the src biomarker proteins of the present invention, or antigenic or immunogenic epitopes thereof, can be, for example, polyclonal or monoclonal antibodies. The present invention also includes chimeric, single chain, and humanized antibodies, as well as Fab, F(ab′)₂, or Fv fragments, or the product of an Fab expression library. Various procedures known in the art may be used for the production of such antibodies and antibody fragments.

Antibodies generated against the polypeptides or peptides corresponding to one or more of the src biomarker sequences of the present invention can be obtained by direct injection of the polypeptides or peptides into an animal, or by administering the polypeptides or peptides to an animal, preferably a nonhuman animal. The antibodies so obtained will then bind to the polypeptides or peptides. In this manner, even a sequence encoding only a fragment of a polypeptide can be used to generate antibodies binding to the whole native polypeptide. Such antibodies can be used, for example, to isolate the polypeptide from tissue expressing that polypeptide.

For the preparation of monoclonal antibodies, any technique which provides antibodies produced by continuous cell line cultures can be used. Examples include the hybridoma technique (Kohler and Milstein, 1975, Nature, 256:495-497), the trioma technique, the human B-cell hybridoma technique (Kozbor et al., 1983, Immunol. Today, 4:72), and the EBV-hybridoma technique to produce human monoclonal antibodies (Cole et al., 1985. In: Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to immunogenic polypeptide products of this invention. Also, transgenic mice may be used to express humanized antibodies to immunogenic polypeptide products of this invention.

An ELISA assay initially involves preparing an antibody specific to antigens of the src biomarker proteins or polypeptides, preferably a monoclonal antibody. In addition, a reporter antibody is used which recognizes and binds to the monoclonal antibody. To the reporter antibody is attached a detectable reagent such as a radioactive isotope, a fluorescent moiety, or, in this example, an enzyme, such as horseradish peroxidase.

To carry out the ELISA assay, a sample is removed from a host, e.g., a patient sample, and incubated on a solid support, e.g., wells of a microtiter plate, or a polystyrene dish, to which the proteins in the sample can bind. Any free protein binding sites on the dish are then blocked by incubating with a non-specific protein such as bovine serum albumin. The monoclonal antibody is then added to the solid support, e.g., the wells or the dish, and allowed to incubate. During the incubation time, the monoclonal antibodies attach to any src biomarker proteins or polypeptides that have attached to the polystyrene dish. All unbound monoclonal antibody is washed away using an appropriate buffer solution. The reporter antibody, e.g., linked to horseradish peroxidase, is added to the support, thereby resulting in the binding of the reporter antibody to any monoclonal antibody which has bound to src biomarker proteins or polypeptides that are present in the sample. Unattached reporter antibody is then washed away. Peroxidase substrate is added to the support and the amount of color developed in a given time period provides a measurement of the amount of src biomarker proteins or polypeptides that are present in a given volume of patient sample when compared against a standard curve.

The present invention encompasses polypeptides comprising, or alternatively, consisting of, an epitope of the polypeptide having an amino acid sequence of one or more of the src biomarker amino acid sequences as set forth in Tables 3-6. The present invention further encompasses polynucleotide sequences encoding an epitope of a polypeptide sequence of src biomarkers of the invention.

The term “epitopes” as used herein, refers to portions of a polypeptide having antigenic or immunogenic activity in an animal, preferably a mammal, and most preferably a human. In a preferred embodiment, the present invention encompasses a polypeptide comprising an epitope, as well as the polynucleotide encoding this polypeptide. An “immunogenic epitope” as used herein, refers to a portion of a protein that elicits an antibody response in an animal, as determined by any method known in the art, for example, by the methods for generating antibodies described infra. (See, for example, Geysen et al., 1983, Proc. Natl. Acad. Sci. USA, 81:3998-4002). The term “antigenic epitope” as used herein refers to a portion of a protein to which an antibody can immunospecifically bind to its antigen as determined by any method well known in the art, for example, by the immunoassays described herein. Immunospecific binding excludes non-specific binding, but does not necessarily exclude cross-reactivity with other antigens. Antigenic epitopes need not necessarily be immunogenic. Either the full-length protein or an antigenic peptide fragment can be used. Antibodies are preferably prepared from these regions or from discrete fragments in regions of the src biomarker nucleic acid and protein sequences comprising an epitope.

Moreover, antibodies can also be prepared from any region of the polypeptides and peptides of the src biomarkers as described herein. A preferred fragment generates the production of an antibody that diminishes or completely prevents ligand binding. In addition, antibodies can be developed against an entire receptor or portions of the receptor, for example, the intracellular carboxy terminal domain, the amino terminal extracellular domain, the entire transmembrane domain, specific transmembrane segments, any of the intracellular or extracellular loops, or any portions of these regions. Antibodies can also be developed against specific functional sites, such as -the site of ligand binding, or sites that are glycosylated, phosphorylated, myristylated, or amidated, for example.

Polypeptide or peptide fragments that function as epitopes may be produced by any conventional means. (See, e.g., Houghten, 1985, Proc. Natl. Acad. Sci. USA, 82:5131-5135; and as described in U.S. Pat. No. 4,631,211).

In the present invention, antigenic epitopes preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Additional non-exclusive preferred antigenic epitopes include the antigenic epitopes disclosed herein, as well as portions thereof, as well as any combination of two, three, four, five or more of these antigenic epitopes. Antigenic epitopes are useful, for example, to raise antibodies, including monoclonal antibodies, that specifically bind the epitope. In addition, antigenic epitopes can be used as the target molecules in immunoassays. (See, for instance, Wilson et al., 1984, Cell, 37:767-778; and Sutcliffe et al., 1983, Science, 219:660-666). Such fragments as described herein are not to be construed, however, as encompassing any fragments which may be disclosed prior to the invention.

Similarly, immunogenic epitopes can be used, for example, to induce antibodies according to methods well known in the art. (See, for instance, Sutcliffe et al., supra; Wilson et al., supra; Chow et al., 1985, Proc. Natl. Acad. Sci. USA, 82:910-914; and Bittle et al., 1985, J. Gen. Virol., 66:2347-2354). Preferred immunogenic epitopes include the immunogenic epitopes disclosed herein, as well as any combination of two, three, four, five or more of these immunogenic epitopes.

Src biomarker polypeptides comprising one or more immunogenic epitopes which elicit an antibody response can be introduction together with a carrier protein, such as an albumin, to an animal system (such as rabbit or mouse). Alternatively, if the polypeptide is of sufficient length (e.g., at least about 25 amino acids), the polypeptide can be presented without a carrier. However, immunogenic epitopes comprising as few as 5 to 10 amino acids have been shown to be sufficient to raise antibodies capable of binding to, at the very least, linear epitopes in a denatured polypeptide (e.g., in Western blotting).

Epitope-bearing polypeptides of the present invention may be used to induce antibodies according to methods well known in the art including, but not limited to, in vivo immunization, in vitro immunization, and phage display methods. See, e.g., Sutcliffe et al., supra; Wilson et al., supra; and Bittle et al., supra). If in vivo immunization is used, animals can be immunized with free peptide; however, the anti-peptide antibody titer may be boosted by coupling the peptide to a macromolecular carrier, such as keyhole limpet hemacyanin (KLH), or tetanus toxoid (TT). For instance, peptides containing cysteine residues can be coupled to a carrier using a linker such as maleimidobenzoyl-N-hydroxysuccinimide ester (MBS), while other peptides may be coupled to carriers using a more general linking agent, such as glutaraldehyde.

Epitope bearing peptides of the invention may also be synthesized as multiple antigen peptides (MAPs), first described by J. P. Tam et al., 1995, Biomed. Pept., Proteins, Nucleic Acids, 199, 1(3):123-32; and Calvo, et al., 1993, J. Immunol., 150(4):1403-12), which are hereby incorporated by reference in their entirety herein. MAPs contain multiple copies of a specific peptide attached to a non-immunogenic lysine core. MAP peptides usually contain four or eight copies of the peptide, which are often referred to as MAP4 or MAP8 peptides. By way of non-limiting example, MAPs can be synthesized onto a lysine core matrix attached to a polyethylene glycol-polystyrene (PEG-PS) support. The peptide of interest is synthesized onto the lysine residues using 9-fluorenylmethoxycarbonyl (Fmoc) chemistry. For example, Applied Biosystems (Foster City, Calif.) offers commercially available MAP resins, such as, for example, the Fmoc Resin 4 Branch and the Fmoc Resin 8 Branch which can be used to synthesize MAPs. Cleavage of MAPs from the resin is performed with standard trifloroacetic acid (TFA)-based cocktails known in the art. Purification of MAPs, except for desalting, is not generally necessary. MAP peptides can be used in immunizing vaccines which elicit antibodies that recognize both the MAP and the native protein from which the peptide was derived.

Epitope-bearing peptides of the invention can also be incorporated into a coat protein of a virus, which can then be used as an immunogen or a vaccine with which to immunize animals, including humans, in order stimulate the production of anti-epitope antibodies. For example, the V3 loop of the gp120 glycoprotein of the human immunodeficiency virus type 1 (HIV-1) has been engineered to be expressed on the surface of rhinovirus. Immunization with rhinovirus displaying the V3 loop peptide yielded apparently effective mimics of the HIV-1 immunogens (as measured by their ability to be neutralized by anti-HIV-1 antibodies as well as by their ability to elicit the production of antibodies capable of neutralizing HIV-1 in cell culture). This techniques of using engineered viral particles as immunogens is described in more detail in Smith et al., 1997, Behring Inst Mitt Feb, (98):229-39; Smith et al., 1998, J. Virol., 72:651-659; and Zhang et al., 1999, Biol. Chem., 380:365-74), which are hereby incorporated by reference herein in their entireties.

Epitope bearing polypeptides of the invention can be modified, for example, by the addition of amino acids at the amino- and/or carboxy-terminus of the peptide. Such modifications are performed, for example, to alter the conformation of the epitope bearing polypeptide such that the epitope will have a conformation more closely related to the structure of the epitope in the native protein. An example of a modified epitope-bearing polypeptide of the invention is a polypeptide in which one or more cysteine residues have been added to the polypeptide to allow for the formation of a disulfide bond between two cysteines, thus resulting in a stable loop structure of the epitope-bearing polypeptide under non-reducing conditions. Disulfide bonds can form between a cysteine residue added to the polypeptide and a cysteine residue of the naturally-occurring epitope, or between two cysteines which have both been added to the naturally-occurring epitope-bearing polypeptide.

In addition, it is possible to modify one or more amino acid residues of the naturally-occurring epitope-bearing polypeptide by substitution with cysteines to promote the formation of disulfide bonded loop structures. Cyclic thioether molecules of synthetic peptides can be routinely generated using techniques known in the art, e.g., as described in PCT publication WO 97/46251, incorporated in its entirety by reference herein. Other modifications of epitope-bearing polypeptides contemplated by this invention include biotinylation.

For the production of antibodies in vivo, host animals, such as rabbits, rats, mice, sheep, or goats, are immunized with either free or carrier-coupled peptides or MAP peptides, for example, by intraperitoneal and/or intradermal injection. Injection material is typically an emulsion containing about 100 μg of peptide or carrier protein and Freund's adjuvant, or any other adjuvant known for stimulating an immune response. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal can be increased by selection of anti-peptide antibodies, e.g., by adsorption of the peptide onto a solid support and elution of the selected antibodies according to methods well known in the art.

As one having skill in the art will appreciate, and as discussed above, the src biomarker polypeptides of the present invention, which include the following: e.g., members of the Src.family of tyrosine kinases, such as Src, Fgr, Fyn, Yes, Blk, Hck, Lck and Lyn, as well as other protein tyrosine kinases, including, Bcr-abl, Jak, PDGFR, c-kit and Ephr, which comprise an immunogenic or antigenic epitope, can be fused to other polypeptide sequences. For example, the polypeptides of the present invention can be fused with the constant domain of immunoglobulins (IgA, IgE, IgG, IgD, or IgM), or portions thereof, e.g., CH1, CH2, CH3, or any combination thereof, and portions thereof, or with albumin (including, but not limited to, recombinant human albumin, or fragments or variants thereof (see, e.g., U.S. Pat. No. 5,876,969; EP Patent No. 0 413 622; and U.S. Pat. No. 5,766,883, incorporated by reference in their entirety herein), thereby resulting in chimeric polypeptides. Such fusion proteins may facilitate purification and may increase half-life in vivo. This has been shown for chimeric proteins containing the first two domains of the human CD4-polypeptide and various domains of the constant regions of the heavy or light chains of mammalian immunoglobulins. See, e.g., Traunecker et al., 1988, Nature, 331:84-86).

Enhanced delivery of an antigen across the epithelial barrier to the immune system has been demonstrated for antigens (e.g., insulin) conjugated to an FcRn binding partner, such as IgG or Fc fragments (see, e.g., PCT Publications WO 96/22024 and WO 99/04813). IgG fusion proteins that have a disulfide-linked dimeric structure due to the IgG portion disulfide bonds have also been found to be more efficient in binding and neutralizing other molecules than are monomeric polypeptides, or fragments thereof, alone. See, e.g., Fountoulakis et al., 1995, J. Biochem., 270:3958-3964).

Nucleic acids encoding epitopes can also be recombined with a gene of interest as an epitope tag (e.g., the hemagglutinin (“HA”) tag or flag tag) to aid in detection and purification of the expressed polypeptide. For example, a system for the ready purification of non-denatured fusion proteins expressed in human cell lines has been described by Janknecht et al., (1991, Proc. Natl. Acad. Sci. USA, 88:8972-897). In this system, the gene of interest is subcloned into a vaccinia recombination plasmid such that the open reading frame of the gene is translationally fused to an amino-terminal tag having six histidine residues. The tag serves as a matrix binding domain for the fusion protein. Extracts from cells infected with the recombinant vaccinia virus are loaded onto an Ni²⁺ nitriloacetic acid-agarose column and histidine-tagged proteins are selectively eluted with imidazole-containing buffers.

Additional fusion proteins of the invention can be generated by employing the techniques of gene-shuffling, motif-shuffling, exon-shuffling, and/or codon-shuffling (collectively referred to as “DNA shuffling”). DNA shuffling can be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., 1997, Curr. Opinion Biotechnol., 8:724-33; Harayama, 1998, Trends Biotechnol., 16(2):76-82; Hansson, et al., 1999, J. Mol. Biol., 287:265-76; and Lorenzo and Blasco, 1998, Biotechniques, 24(2):308-313, the contents of each of which are hereby incorporated by reference in its entirety).

In an embodiment of the invention, alteration of polynucleotides corresponding to one or more of the src biomarker polynucleotide sequences as set forth in Tables 3-6, and the polypeptides encoded by these polynucleotides, can be achieved by DNA shuffling. DNA shuffling involves the assembly of two or more DNA segments by homologous or site-specific recombination to generate variation in the polynucleotide sequence. In another embodiment, polynucleotides of the invention, or their encoded polypeptides, may be altered by being subjected to random mutapolynucleotides and polypeptidesis by error-prone PCR, random nucleotide insertion, or other methods, prior to recombination. In another embodiment, one or more components, motifs, sections, parts, domains, fragments, etc., of a polynucleotide encoding a polypeptide of this invention may be recombined with one or more components, motifs, sections, parts, domains, fragments, etc. of one or more heterologous molecules.

Another aspect of the present invention relates to antibodies and T-cell antigen receptors (TCRs), which immunospecifically bind to a polypeptide, polypeptide fragment, or variant one or more of the src biomarker amino acid sequences as set forth in Tables 3-6, and/or an epitope thereof, of the present invention (as determined by immunoassays well known in the art for assaying specific antibody-antigen binding).

The basic antibody structural unit of an antibody or immunoglobulin is known to comprise a tetramer. Each tetramer is composed of two identical pairs of polypeptide chains, each pair having one “light” (about 25 kDa) and one “heavy” chain (about 50-70 kDa). The amino terminal portion of each chain includes a variable region of about 100 to 110 or more amino acids; the variable region is primarily responsible for antigen recognition. The carboxy terminal portion of each chain defines a constant region that is primarily responsible for immunoglobulin effector function. Immunoglobulin light chains, including human light chains, are of the kappa and lambda types. Immunoglobulin heavy chain isotypes include IgM, IgD, IgG, IgA, and IgE. (See, generally, Fundamental Immunology, Ch. 7, Paul, W., Ed., 2nd Ed. Raven Press, N.Y. (1989), incorporated herein by reference in its entirety). The variable regions of each light/heavy chain pair form the antibody or immunoglobulin binding site. Thus, for example, an intact IgG antibody has two binding sites. Except in bifunctional or bispecific antibodies, the two binding sites are the same.

The chains of an immunoglobulin molecule exhibit the same general structure of relatively conserved framework regions (FR) joined by three hypervariable regions, also called complementarity determining regions or CDRs. The CDRs of the heavy and the light chains of each pair are aligned by the framework regions, thus enabling binding to a specific epitope. From N-terminus to C-terminus, both the light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)); Chothia & Lesk, 1987, J. Mol. Biol., 196:901-917; or Chothia et al., 1989, Nature, 342:878-883.

A bispecific or bifunctional antibody is an artificial hybrid antibody having two different heavy/light chain pairs and two different binding sites. Bispecific antibodies can be produced by a variety of methods, including fusion of hybridomas or linking of Fab′ fragments. (See, e. g., Songsivilai & Lachmann , 1990, Clin. Exp. Immunol., 79:315-321; Kostelny et al., 1992, J. Immunol., 148:1547 1553). In addition, bispecific antibodies can be formed as “diabodies” (See, Holliger et al., 1993, Proc. Natl. Acad. Sci. USA, 90:6444-6448), or “Janusins” (See, Traunecker et al., 1991, EMBO J., 10:3655-3659 and Traunecker et al., 1992, Int. J. Cancer Suppl. 7:51-52-127).

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), intracellularly made antibodies (i.e., intrabodies), and epitope-binding fragments of any of the above. The term “antibody”, as used herein, refers to immunoglobulin molecules and immunologically active portions or fragments of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class or subclass (e.g., IgG1, IgG2, IgG3, IgG4, IgA1 and IgA2) of immunoglobulin molecule. In a preferred embodiment, the immunoglobulin is an IgG1 isotype. In another preferred embodiment, the immunoglobulin is an IgG2 isotype. In another preferred embodiment, the immunoglobulin is an IgG4 isotype.

Immunoglobulins may have both a heavy and a light chain. An array of IgG, IgE, IgM, IgD, IgA, and IgY heavy chains can be paired with a light chain of the kappa or lambda types. Most preferably, the antibodies of the present invention are human antigen-binding antibodies and antibody fragments and include, but are not limited to, Fab, Fab′ F(ab′) 2, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, can comprise the variable region(s) alone or in combination with the entirety or a portion of the following: hinge region, and CH1, CH2, and CH3 domains. Also included in connection with the invention are antigen-binding fragments also comprising any combination of variable region(s) with a hinge region, and CH1, CH2, and CH3 domains. The antibodies of the invention may be from any animal origin including birds and mammals. Preferably, the antibodies are of human, murine (e.g., mouse and rat), donkey, sheep, rabbit, goat, guinea pig, camel, horse, or chicken origin. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example, in U.S. Pat. No. 5,939,598.

The antibodies of the present invention can be mono specific, bispecific, trispecific, or of greater multispecificity. Multispecific antibodies can be specific for different epitopes of a polypeptide of the present invention, or can be specific for both a polypeptide of the present invention, and a heterologous epitope, such as a heterologous polypeptide or solid support material. (See, e.g., PCT publications WO 93/17715; WO 92/08802; WO 91/00360; WO 92/05793; Tutt et al., 1991, J. Immunol., 147:60-69; U.S. Pat. Nos. 4,474,893; 4,714,681; 4,925,648; 5,573,920; 5,601,819; and Kostelny et al., 1992, J. Immunol., 148:1547-1553).

Antibodies of the present invention can be described or specified in terms of the epitope(s) or portion(s) of a polypeptide of the present invention which they recognize or specifically bind. The epitope(s) or polypeptide portion(s) can be specified, e.g., by N-terminal and C-terminal positions, by size in contiguous amino acid residues, or as presented in the sequences defined in Tables 3-6 herein. Further included in accordance with the present invention are antibodies which bind to polypeptides encoded by polynucleotides which hybridize to a polynucleotide of the present invention under stringent, or moderately stringent, hybridization conditions as described herein.

The antibodies of the invention (including molecules comprising, or alternatively consisting of, antibody fragments or variants thereof) can bind immunospecifically to a polypeptide or polypeptide fragment or variant human src biomarker protein as set forth in Tables 3-6 and/or monkey src biomarker protein.

By way of non-limiting example, an antibody can be considered to bind to a first antigen preferentially if it binds to the first antigen with a dissociation constant (Kd) that is less than the antibody's Kd for the second antigen. In another non-limiting embodiment, an antibody can be considered to bind to a first antigen preferentially if it binds to the first antigen with an affinity that is at least one order of magnitude less than the antibody's Ka for the second antigen. In another non-limiting embodiment, an antibody can be considered to bind to a first antigen preferentially if it binds to the first antigen with an affinity that is at least two orders of magnitude less than the antibody's Kd for the second antigen.

In another nonlimiting embodiment, an antibody may be considered to bind to a first antigen preferentially if it binds to the first antigen with an off rate (koff) that is less than the antibody's koff for the second antigen. In another nonlimiting embodiment, an antibody can be considered to bind to a first antigen preferentially if it binds to the first antigen with an affinity that is at least one order of magnitude less than the antibody's koff for the second antigen. In another nonlimiting embodiment, an antibody can be considered to bind to a first antigen preferentially if it binds to the first antigen with an affinity that is at least two orders of magnitude less than the antibody's koff for the second antigen.

Antibodies of the present invention can also be described or specified in terms of their binding affinity to a src biomarker polypeptide of the present invention, e.g., members of the Src family of tyrosine kinases, for example, Src, Fgr, Fyn, Yes, Blk, Hck, Lck and Lyn, as well as other protein tyrosine kinases, including, Bcr-abl, Jak, PDGFR, c-kit and Ephr. Preferred binding affinities include those with a dissociation constant or Kd of less than 5×10⁻² M, 1×10⁻² M, 5×10⁻³ M, 1×10⁻³ M, 5×10⁻⁴ M, or 1×10⁻⁴ M. More preferred binding affinities include those with a dissociation constant or Kd less than 5×10⁻⁵ M, 1×10⁻⁵ M, 5×10⁻⁶ M, 1×10⁻⁶ M, 5×10⁻⁷ M, 1×10⁻⁷ M, 5×10⁻⁸ M, or 1×10⁻⁸ M. Even more preferred antibody binding affinities include those with a dissociation constant or Kd of less than 5×10⁻⁹ M, 1×10⁻⁹ M, 5×10⁻¹⁰ M, 1×10⁻¹⁰ M, 5×10⁻¹¹ M, 1×10⁻¹¹ M, 5×10⁻¹² M, 1×10⁻¹² M, 5×10⁻¹³ M, 1×10⁻¹³ M, 5×10⁻¹⁴ M, 1×10⁻¹⁴ M, 5×10⁻¹⁵ M, or 1×10⁻¹⁵ M.

In specific embodiments, antibodies of the invention bind to src biomarker polypeptides, or fragments, or variants thereof, with an off rate (koff) of less than or equal to about 5×10⁻² sec⁻¹, 1×10⁻² sec⁻¹, 5×10⁻³ sec⁻¹, or 1×10⁻³ sec⁻¹. More preferably, antibodies of the invention bind to src biomarker protein polypeptides or fragments or variants thereof with an off rate (koff) of less than or equal to about 5×10⁻⁴ sec⁻¹, 1×10⁻⁴ sec⁻¹, 5×10⁻⁵ sec⁻¹, 5×10⁻⁶ sec⁻¹, 1×10⁻⁶ sec⁻¹, 5×10⁻⁷ sec⁻¹, or 1×10⁻⁷ sec¹.

In other embodiments, antibodies of the invention bind to src biomarker polypeptides or fragments or variants thereof with an on rate (kon) of greater than or equal to 1×10³ M⁻¹ sec⁻¹, 5×10³ M⁻¹ sec⁻¹, 1×10⁴ M⁻¹ sec⁻¹. More preferably, antibodies of the invention bind to src biomarker polypeptides or fragments or variants thereof with an on rate greater than or equal to 1×10⁵ M⁻¹ sec⁻¹, 5×10⁵ M⁻¹ sec⁻¹, 1×10⁶ M⁻¹ sec-1, 5×10⁻⁶ M⁻¹ sec⁻¹, or 1×10⁻⁷ M⁻¹ sec⁻¹.

The present invention also provides antibodies that competitively inhibit the binding of an antibody to an epitope of the invention as determined by any method known in the art for determining competitive binding, for example, the immunoassays as described herein. In preferred embodiments, the antibody competitively inhibits binding to an epitope by at least 95%, at least 90%, at least 85%, at least 80%, at least 75%, at least 70%, at least 60%, or at least 50%.

Antibodies of the present invention may act as agonists or antagonists of the src biomarker polypeptides of the present invention. For example, the present invention includes antibodies which disrupt receptor/ligand interactions with polypeptides of the invention either partially or fully. The invention includes both receptor-specific antibodies and ligand-specific antibodies. The invention also includes receptor-specific antibodies which do not prevent ligand binding, but do prevent receptor activation. Receptor activation (i.e., signaling) can be determined by techniques described herein or as otherwise known in the art. For example, receptor activation can be determined by detecting the phosphorylation (e.g., on tyrosine or serine/threonine) of the receptor or its substrate by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in the absence of the antibody.

In another embodiment of the present invention, antibodies that immunospecifically bind to a src biomarker protein or a fragment or variant thereof, comprise a polypeptide having the amino acid sequence of any one of the heavy chains expressed by an anti-src biomarker protein antibody-expressing cell line of the invention, and/or any one of the light chains expressed by an anti-src biomarker protein antibody-expressing cell line of the invention. In another embodiment of the present invention, antibodies that immunospecifically bind to a src biomarker protein or a fragment or variant thereof, comprise a polypeptide having the amino acid sequence of any one of the VH domains of a heavy chain expressed by an anti-src biomarker protein antibody-expressing cell line, and/or any one of the V_{L} domains of a light chain expressed by an anti-src biomarker protein antibody-expressing cell line. In preferred embodiments, antibodies of the present invention comprise the amino acid sequence of a V_{H} domain and V_{L} domain expressed by a single anti-src biomarker protein antibody-expressing cell line. In alternative embodiments, antibodies of the present invention comprise the amino acid sequence of a V_{H} domain and a V_{L} domain expressed by two different anti-src biomarker protein antibody-expressing cell lines.

Molecules comprising, or alternatively consisting of, antibody fragments or variants of the V_{H} and/or V_{L} domains expressed by an anti-src biomarker protein antibody-expressing cell line that immunospecifically bind to a src biomarker protein are also encompassed by the invention, as are nucleic acid molecules encoding these V_{H} and V_{L} domains, molecules, fragments and/or variants.

The present invention also provides antibodies that immunospecificially bind to a polypeptide, or polypeptide fragment or variant of a src biomarker protein, wherein said antibodies comprise, or alternatively consist of, a polypeptide having an amino acid sequence of any one, two, three, or more of the V_{H} CDRs contained in a heavy chain expressed by one or more anti-src biomarker protein antibody expressing cell lines. In particular, the invention provides antibodies that immunospecifically bind to a src biomarker protein, comprising, or alternatively consisting of, a polypeptide having the amino acid sequence of a V_{H} CDR1 contained in a heavy chain expressed by one or more anti-src biomarker protein antibody expressing cell lines. In another embodiment, antibodies that immunospecifically bind to a src biomarker protein, comprise, or alternatively consist of, a polypeptide having the amino acid sequence of a V_{H} CDR2 contained in a heavy chain expressed by one or more anti-src biomarker protein antibody expressing cell lines. In a preferred embodiment, antibodies that immunospecifically bind to a src biomarker protein, comprise, or alternatively consist of, a polypeptide having the amino acid sequence of a V_{H} CDR3 contained in a heavy chain expressed by one or more anti-src biomarker protein antibody expressing cell line of the invention. Molecules comprising, or alternatively consisting of, these antibodies, or antibody fragments or variants thereof, that immunospecifically bind to a src biomarker protein or a Src biomarker protein fragment or variant thereof are also encompassed by the invention, as are nucleic acid molecules encoding these antibodies, molecules, fragments and/or variants.

The present invention also provides antibodies that immunospecificially bind to a polypeptide, or polypeptide fragment or variant of a src biomarker protein, wherein said antibodies comprise, or alternatively consist of, a polypeptide having an amino acid sequence of any one, two, three, or more of the V_{L} CDRs contained in a heavy chain expressed by one or more anti-src biomarker protein antibody expressing cell lines of the invention. In particular, the invention provides antibodies that immunospecifically bind to a src biomarker protein, comprising, or alternatively consisting of, a polypeptide having the amino acid sequence of a V_{L} CDR1 contained in a heavy chain expressed by one or more anti-src biomarker protein antibody-expressing cell lines of the invention. In another embodiment, antibodies that immunospecifically bind to a src biomarker protein, comprise, or alternatively consist of, a polypeptide having the amino acid sequence of a V_{L} CDR2 contained in a heavy chain expressed by one or more anti-src biomarker protein antibody-expressing cell lines of the invention. In a preferred embodiment, antibodies that immunospecifically bind to a src biomarker protein, comprise, or alternatively consist of a polypeptide having the amino acid sequence of a V_{L} CDR3 contained in a heavy chain expressed by one or more anti-src biomarker protein antibody-expressing -cell lines of the invention. Molecules comprising, or alternatively consisting of, these antibodies, or antibody fragments or variants thereof, that immunospecifically bind to a src biomarker protein or a src biomarker protein fragment or variant thereof are also encompassed by the invention, as are nucleic acid molecules encoding these antibodies, molecules, fragments and/or variants.

The present invention also provides antibodies (including molecules comprising, or alternatively consisting of, antibody fragments or variants) that immunospecifically bind to a src biomarker protein polypeptide or polypeptide fragment or variant of a src biomarker protein, wherein the antibodies comprise, or alternatively consist of, one, two, three, or more V_{H} CDRS, and one, two, three or more V_{L} CDRS, as contained in a heavy chain or light chain expressed by one or more anti-src biomarker protein antibody-expressing cell lines of the invention. In particular, the invention provides antibodies that immunospecifically bind to a polypeptide or polypeptide fragment or variant of a src biomarker protein, wherein the antibodies comprise, or alternatively consist of, a V_{H} CDR1 and a V_{L} CDR1, a V_{H} CDR1 and a V_{L} CDR2, a V_{H} CDR1 and a V_{L} CDR3, a V_{H} CDR2 and a V_{L} CDR1, V_{H} CDR2 and V_{L} CDR2, a V_{H} CDR2 and a V_{L} CDR3, a V_{H} CDR3 and a V_{H} CDR1, a V_{H} CDR3 and a V_{L} CDR2, a V_{H} CDR3 and a V_{L} CDR3, or any combination thereof, of the V_{H} CDRs and V_{L} CDRs contained in a heavy chain or light chain immunoglobulin molecule expressed by one or more anti-src biomarker protein antibody-expressing cell lines of the invention. In a preferred embodiment, one or more of these combinations are from a single anti-src biomarker protein antibody-expressing cell line of the invention. Molecules comprising, or alternatively consisting of, fragments or variants of these antibodies that immunospecifically bind to the src biomarker proteins are also encompassed by the invention, as are nucleic acid molecules encoding these antibodies, molecules, fragments or variants.

The present invention also provides nucleic acid molecules, generally isolated, encoding an, antibody of the invention (including molecules comprising, or alternatively consisting of, antibody fragments or variants thereof). In a specific embodiment, a nucleic acid molecule of the invention encodes an antibody (including molecules comprising, or alternatively consisting of, antibody fragments or variants thereof), comprising, or alternatively consisting of, a V_{H} domain having an amino acid sequence of any one of the V_{H} domains of a heavy chain expressed by an anti-src biomarker protein antibody-expressing cell line of the invention and a V_{L} domain having an amino acid sequence of a light chain expressed by an anti-src biomarker protein antibody-expressing cell line of the invention. In another embodiment, a nucleic acid molecule of the invention encodes an antibody (including molecules comprising, or alternatively consisting of, antibody fragments or variants thereof), comprising, or alternatively consisting of, a V_{H} domain having an amino acid sequence of any one of the V_{H} domains of a heavy chain expressed by an anti-src biomarker protein antibody-expressing cell line of the invention, or a V_{L} domain having an amino acid sequence of a light chain expressed by an anti-Src biomarker protein antibody-expressing cell line of the invention.

The present invention also provides antibodies that comprise, or alternatively consist of, variants (including derivatives) of the antibody molecules (e.g., the V_{H} domains and/or V_{L} domains) described herein, which antibodies immunospecifically bind to a src biomarker protein or fragment or variant thereof.

Standard techniques known to those of skill in the art can be used to introduce mutations in the nucleotide sequence encoding a molecule of the invention, including, for example, site-directed mutapolynucleotides and polypeptidesis and PCR-mediated mutapolynucleotides and polypeptidesis which result in amino acid substitutions. Preferably the molecules are immunoglobulin molecules. Also, preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions, relative to the reference V_{H} domain, V_{H} CDR1, V_{H} CDR2, V_{H} CDR3, V_{L} domain, V_{L} CDR1, V_{L} CDR2, or V_{L} CDR3.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutapolynucleotides and polypeptidesis. The resultant mutants can be screened for biological activity to identify mutants that retain activity.

For example, it is possible to introduce mutations only in framework regions or only in CDR regions of an antibody molecule. Introduced mutations can be silent or neutral missense mutations, i.e., have no, or little, effect on an antibody's ability to bind antigen. These types of mutations can be useful to optimize codon usage, or to improve hybridoma antibody production. Alternatively, non-neutral missense mutations can alter an antibody's ability to bind antigen. The location of most silent and neutral missense mutations is likely to be in the framework regions, while the location of most non-neutral missense mutations is likely to be in the CDRS, although this is not an absolute requirement. One of skill in the art is able to design and test mutant molecules with desired properties, such as no alteration in antigen binding activity or alteration in binding activity (e.g., improvements in antigen binding activity or change in antibody specificity). Following mutapolynucleotides and polypeptidesis, the encoded protein may routinely be expressed and the functional and/or biological activity of the encoded protein can be determined using techniques described herein or by routinely modifying techniques known and practiced in the art.

In a specific embodiment, an antibody of the invention (including a molecule comprising, or alternatively consisting of, an antibody fragment or variant thereof), that immunospecifically binds to src biomarker polypeptides or fragments or variants thereof, comprises, or alternatively consists of, an amino acid sequence encoded by a nucleotide sequence that hybridizes to a nucleotide sequence that is complementary to that encoding one of the V_{H} or V_{L} domains expressed by one or more anti-src biomarker protein antibody-expressing cell lines of the invention, preferably under stringent conditions, e.g., hybridization to filter-bound DNA in 6× sodium chloride/sodium citrate (SSC) at about 45° C. followed by one or more washes in 0.2×SSC/0.1% SDS at about 50-65° C., preferably under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3). Nucleic acid molecules encoding these antibodies are also encompassed by the invention.

It is well known within the art that polypeptides, or fragments or variants thereof, with similar amino acid sequences often have similar structure and many of the same biological activities. Thus, in one embodiment, an antibody (including a molecule comprising, or alternatively consisting of, an antibody fragment or variant thereof), that immunospecifically binds to a src biomarker polypeptide or fragments or variants of a src biomarker polypeptide, comprises, or alternatively consists of, a V_{H} domain having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a V_{H} domain of a heavy chain expressed by an anti-src biomarker protein antibody-expressing cell line of the invention.

In another embodiment, an antibody (including a molecule comprising, or alternatively consisting of, an antibody fragment or variant thereof), that immunospecifically binds to a src biomarker polypeptide or fragments or variants of a src biomarker protein polypeptide, comprises, or alternatively consists of, a V_{L} domain having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a V_{L} domain of a light chain expressed by an anti-src biomarker protein antibody-expressing cell line of the invention.

The present invention also provides antibodies (including molecules comprising, or alternatively consisting of, antibody fragments or variants thereof), that down-regulate the cell-surface expression of a src biomarker protein, as determined by any method known in the art such as, for example, FACS analysis or immunofluorescence assays. By way of a non-limiting hypothesis, such down-regulation may be the result of antibody induced internalization of src biomarker protein. Such antibodies can comprise, or alternatively consist of, a portion (e.g., V_{H} CDR1, V_{H} CDR2, V_{H} CDR3, V_{L} CDR1, V_{L} CDR2, or V_{L} CDR3) of a V_{H} or V_{L} domain having an amino acid sequence of an antibody of the invention, or a fragment or variant thereof.

In another embodiment, an antibody that down-regulates the cell-surface expression of a src biomarker protein comprises, or alternatively consists of, a polypeptide having the amino acid sequence of a V_{H} domain of an antibody of the invention, or a fragment or variant thereof and a V_{L} domain of an antibody of the invention, or a fragment or variant thereof. In another embodiment, an antibody that down-regulates the cell-surface expression of a src biomarker protein comprises, or alternatively consists of, a polypeptide having the amino acid sequence of a V_{H} domain and a V_{L} domain from a single antibody (or scFv or Fab fragment) of the invention, or fragments or variants thereof. In another embodiment, an antibody that down-regulates the cell-surface expression of a src biomarker protein comprises, or alternatively consists of, a polypeptide having the amino acid sequence of a V_{H} domain of an antibody of the invention, or a fragment or variant thereof. In another embodiment, an antibody that down-regulates the cell-surface expression of a src biomarker protein comprises, or alternatively consists of, a polypeptide having the amino acid sequence of a V_{L} domain of an antibody of the invention, or a fragment or variant thereof.

In a preferred embodiment, an antibody that down-regulates the cell-surface expression of a src biomarker protein comprises, or alternatively consists of, a polypeptide having the amino acid sequence of a V_{H} CDR3 of an antibody of the invention, or a fragment or variant thereof. In another preferred embodiment, an antibody that down-regulates the cell-surface expression of a src biomarker protein comprises, or alternatively consists of, a polypeptide having the amino acid sequence of a V_{L} CDR3 of an antibody of the invention, or a fragment or variant thereof. Nucleic acid molecules encoding these antibodies are also encompassed by the invention.

In another preferred embodiment, an antibody that enhances the activity of a src biomarker protein, or a fragment or variant thereof, comprises, or alternatively consists of, a polypeptide having the amino acid sequence of a V_{L} CDR3 of an antibody of the invention, or a fragment or variant thereof. Nucleic acid molecules encoding these antibodies are also encompassed by the invention.

As nonlimiting examples, antibodies of the present invention can be used to purify, detect, and target the polypeptides of the present invention, including both in vitro and in vivo diagnostic, detection, screening, and/or therapeutic methods. For example, the antibodies have use in immunoassays for qualitatively and quantitatively measuring levels of the src biomarker polypeptides of the present invention in biological samples. (See, e.g., Harlow et al., Antibodies: A Laboratory Manual, Cold Spring Harbor Laboratory Press, 2nd Ed. 1988, which is incorporated by reference herein in its entirety).

By way of another nonlimiting example, antibodies of the invention can be administered to individuals as a form of passive immunization. Alternatively, antibodies of the present invention can be used for epitope mapping to identify the epitope(s) that are bound by the antibody. Epitopes identified in this way can, in turn, for example, be used as vaccine candidates, i.e., to immunize an individual to elicit antibodies against the naturally-occurring forms of one or more src biomarker proteins.

As discussed in more detail below, the antibodies of the present invention can be used either alone or in combination with other compositions. The antibodies can further be recombinantly fused to a heterologous polypeptide at the N- or C-terminus, or chemically conjugated (including covalent and non-covalent conjugations) to polypeptides or other compositions. For example, antibodies of the present invention can be recombinantly fused or conjugated to molecules that are useful as labels in detection assays and to effector molecules such as heterologous polypeptides, drugs, radionuclides, or toxins. See, e.g., PCT publications WO 92/08495; WO 91/14438; WO 89/12624; U.S. Pat. No. 5,314,995 and EP 396, 387.

The antibodies of the invention include derivatives that are modified, i.e., by the covalent attachment of any type of molecule to the antibody. For example, without limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative can contain one or more non-classical amino acids.

The antibodies of the present invention may be generated by any suitable method known in the art. Polyclonal antibodies directed against an antigen or immunogen of interest can be produced by various procedures well known in the art. For example, a src biomarker polypeptide or peptide of the invention can be administered to various host animals as elucidated above to induce the production of sera containing polyclonal antibodies specific for the antigen. Various adjuvants may be used to increase the immunological response, depending on the host species; adjuvants include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art, including the use of hybridoma, recombinant and phage display technologies, or a combination thereof. For example, monoclonal antibodies can be produced using hybridoma techniques as known and practiced in the art and as taught, for example, in Harlow et al., Antibodies: A Laboratory Manual, (Cold Spring Harbor Laboratory Press, 2nd Ed. 1988; Hammerling, et al., In: Monoclonal Antibodies and T-Cell Hybridomas, Elsevier, N.Y., pages 563-681, 1981, the contents of which are incorporated herein by reference in their entireties. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In a nonlimiting example, mice can be immunized with a polypeptide or peptide of the invention, or with a cell expressing the polypeptide or peptide. Once an immune response is detected, e.g., antibodies specific for the antigen are detected in the sera of immunized mice, the spleen is harvested and splenocytes are isolated. The splenocytes are then fused by well known techniques to any suitable myeloma cells, for example cells from cell line SP2/0 or P3X63-AG8.653 available from the ATCC. Hybridomas are selected and cloned by limiting dilution techniques. The hybridoma clones are then assayed by methods known in the art to determine and select those cells that secrete antibodies capable of binding to a polypeptide of the invention. Ascites fluid, which generally contains high levels of antibodies, can be generated by immunizing mice with positive hybridoma clones.

Accordingly, the present invention encompasses methods of generating monoclonal antibodies, as well as the antibodies produced by these methods, comprising culturing a hybridoma cell secreting an antibody of the invention wherein, preferably, the hybridoma is generated by fusing splenocytes isolated from a mouse immunized with a src biomarker polypeptide or peptide antigen of the invention with myeloma cells and then screening the hybridomas resulting from the fusion for hybridoma clones that secrete an antibody that binds to a polypeptide of the invention.

Another well known method for producing both polyclonal and monoclonal human B cell lines is transformation using Epstein Barr Virus (EBV). Protocols for generating EBV-transformed B cell lines are commonly known in the art, such as, for example, the protocol outlined in Chapter 7.22 of Current Protocols in Immunology, Coligan et al., Eds., 1994, John Wiley & Sons, NY, which is hereby incorporated by reference herein in its entirety. The source of B cells for transformation is commonly human peripheral blood, but B cells for transformation can also be obtained from other sources including, but not limited to, lymph node, tonsil, spleen, tumor tissue, and infected tissues. Tissues are generally prepared as single cell suspensions prior to EBV transformation. In addition, T cells that may be present in the B cell samples can be either physically removed or inactivated (e.g., by treatment with cyclosporin A). The removal of T cells is often advantageous, because T cells from individuals seropositive for anti-EBV antibodies can suppress B cell immortalization by EBV. In general, a sample containing human B cells is innoculated with EBV and cultured for 3-4 weeks. A typical source of EBV is the culture supernatant of the B95-8 cell line (ATCC; VR-1492). Physical signs of EBV transformation can generally be seen toward the end of the 3-4 week culture period.

By phase-contrast microscopy, transformed cells appear large, clear and “hairy”; they tend to aggregate in tight clusters of cells. Initially, EBV lines are generally polyclonal. However, over prolonged periods of cell culture, EBV lines can become monoclonal as a result of the selective outgrowth of particular B cell clones. Alternatively, polyclonal EBV transformed lines can be subcloned (e.g., by limiting dilution) or fused with a suitable fusion partner and plated at limiting dilution to obtain monoclonal B cell lines. Suitable fusion partners for EBV transformed cell lines include mouse myeloma cell lines (e.g., SP2/0, X63-Ag8.653), heteromyeloma cell lines (human x mouse; e.g., SPAM-8, SBC-H20, and CB-F7), and human cell lines (e.g., GM 1500, SKO-007, RPMI 8226, and KR-4). Thus, the present invention also includes a method of generating polyclonal or monoclonal human antibodies against polypeptides of the invention or fragments thereof, comprising EBV-transformation of human B cells.

Antibody fragments that recognize specific epitopes can be generated by known techniques. For example, Fab and F(ab′)2 fragments of the invention may be produced by proteolytic cleavage of immunoglobulin molecules, using enzymes such as papain (to produce Fab fragments) or pepsin (to produce F (ab′) 2 fragments). F(ab′)2 fragments contain the variable region, the light chain constant region and the CH1 domain of the heavy chain.

Antibodies encompassed by the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds to the antigen of interest can be selected or identified with antigen, e.g., using labeled antigen or antigen bound or captured onto a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., 1995, J. Immunol. Methods, 182:41-50; Ames et al., 1995, J. Immunol. Methods, 184:177-186; Kettleborough et al., 1994, Eur. J. Immunol., 24:952-958; Persic et al., 1997, Gene, 187:9-18; Burton et al., 1994, Advances in Immunology, 57:191-280; PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., 1992, BioTechniques, 12(6):864-869; Sawai et al., 1995, AJRI, 34:2634; and Better et al., 1988, Science, 240:1041-1043, which are hereby incorporated by reference herein in their entireties.

Examples of techniques that can be used to produce single-chain Fvs and antibodies include those described in U.S. Pat. Nos. 4,946,778 and 5,258,498; Huston et al., 1991, Methods in Enzymology, 203:46-88; Shu et al., 1993, Proc. Natl. Acad. Sci. USA, 90:7995-7999; and Skerra et al., 1988, Science, 240:1038-1040. For some uses, including the in vivo use of antibodies in humans and in in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. (See, e.g., Morrison, 1985, Science, 229:1202; Oi et al., 1986, BioTechniques, 4:214; Gillies et al., 1989, J. Immunol. Methods, 125:191-202; and U.S. Pat. Nos. 5,807,715; 4,816,567; and 4,816,397, which are incorporated herein by reference in their entirety).

Humanized antibodies are antibody molecules from non-human species antibody that bind to the desired antigen and have one or more complementarity determining regions (CDRs) from the nonhuman species and framework regions from a human immunoglobulin molecule. Often, framework residues in the human framework regions are substituted with the corresponding residues from the CDR donor antibody to alter, preferably improve, antigen binding. These framework substitutions are identified by methods well known in the art, e.g., by modeling of the interactions of the CDR and framework residues to identify framework residues important for antigen binding, and by sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties). Antibodies can be humanized using a variety of techniques known in the art, including, for example, CDR-grafting (EP 239,400; PCT publication WO 91/09967; U.S. Pat. Nos. 5,225,539; 5,530,101; and 5,585,089); veneering or resurfacing (EP 592,106; EP 519,596; Padlan, 1991, Molecular Immunology, 28(4/5):489-498; Studnicka et al., 1994, Protein Engineering, 7(6):805-814; Roguska et al., 1994, Proc. Natl. Acad. Sci. USA, 91:969-973; and chain shuffling (U.S. Pat. No. 5,565,332).

Completely human antibodies can be made by a variety of methods known in the art, including the phage display methods described above, using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Completely human antibodies are particularly desirable for therapeutic treatment of human patients, so as to avoid or alleviate immune reaction to foreign protein. Human antibodies can be made by a variety of methods known in the art, including the phage display methods described above, using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin polynucleotides and polypeptides. For example, the human heavy and light chain immunoglobulin gene complexes can be introduced randomly, or by homologous recombination, into mouse embryonic stem cells. Alternatively, the human variable region, constant region, and diversity region may be introduced into mouse embryonic stem cells, in addition to the human heavy and light chain polynucleotides and polypeptides. The mouse heavy and light chain immunoglobulin polynucleotides and polypeptides can be rendered nonfunctional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention.

Monoclonal antibodies directed against the antigen can be obtained from the immunized transgenic mice using conventional hybridoma technology. The human immunoglobulin transpolynucleotides and polypeptides harbored by the transgenic mice rearrange during B cell differentiation, and subsequently undergo class switching and somatic mutation.

Thus, using such a technique, it is possible to produce useful human IgG, IgA, IgM and IgE antibodies. For an overview of the technology for producing human antibodies, see Lonberg and Huszar, 1995, Intl. Rev. Immunol., 13:65-93. For a detailed discussion of the technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; 5,939,598; 6,075,181; and 6,114,598, which are incorporated by reference herein in their entirety. In addition, companies such as Abgenix, Inc. (Fremont, Calif.) and Genpharm (San Jose, Calif.) can be engaged to provide human antibodies directed against a selected antigen using technology similar to the above described technologies.

Completely human antibodies which recognize a selected epitope can be generated using a technique referred to as “guided selection”. In this approach, a selected non-human monoclonal antibody, e.g., a mouse antibody, is used to guide the selection of a completely human antibody recognizing the same epitope. (Jespers et al., 1988, BioTechnology, 12:899-903).

Further, antibodies to the polypeptides of the invention can, in turn, be utilized to generate anti-idiotypic antibodies that “mimic” src biomarker polypeptides of the invention using techniques well known to those skilled in the art. (See, e.g., Greenspan and Bona, 1989, FASEB J., 7(5):437-444 and Nissinoff, 1991, J. Immunol., 147(8):2429-2438). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize the polypeptide and/or its ligand, e.g., in therapeutic regimens. Such neutralizing anti-idiotypes or Fab fragments of such anti-idiotypes can be used to neutralize polypeptide ligand. For example, such anti-idiotypic antibodies can be used to bind a polypeptide of the invention and/or to bind its ligands/receptors, and thereby activate or block its biological activity.

Intrabodies are antibodies, often scFvs, that are expressed from a recombinant nucleic acid molecule and are engineered to be retained intracellularly (e.g., retained in the cytoplasm, endoplasmic reticulum, or periplasm of the host cells). Intrabodies can be used, for example, to ablate the function of a protein to which the intrabody binds. The expression of intrabodies can also be regulated through the use of inducible promoters in the nucleic acid expression vector comprising nucleic acid encoding the intrabody. Intrabodies of the invention can be produced using methods known in the art, such as those disclosed and reviewed in Chen et al., 1994, Hum. Gene Ther., 5:595-601; Marasco, W. A., 1997, Gene Ther., 4:11-15; Rondon and Marasco, 1997, Annu. Rev. Microbiol., 51:257-283; Proba et al., 1998, J. Mol. Biol., 275:245-253; Cohen et al., 1998, Oncogene, 17:2445-2456; Ohage and Steipe, 1999, J. Mol. Biol., 291:1119-1128; Ohage et al., 1999, J. Mol. Biol., 291:1129-1134; Wirtz and Steipe, 1999, Protein Sci., 8:2245-2250; Zhu et al., 1999, J. Immunol. Methods, 231:207-222.

XenoMouse Technology Antibodies in accordance with the invention are preferably prepared by the utilization of a transgenic mouse that has a substantial portion of the human antibody producing genome inserted, but that is rendered deficient in the production of endogenous murine antibodies (e.g., XenoMouse strains available from Abgenix Inc., Fremont, Calif.). Such mice are capable of producing human immunoglobulin molecules and antibodies and are virtually deficient in the production of murine immunoglobulin molecules and antibodies. Technologies utilized for achieving the same are disclosed in the patents, applications, and references disclosed herein.

The ability to clone and reconstruct megabase-sized human loci in YACs and to introduce them into the mouse gernline provides a powerful approach to elucidating the functional components of very large or crudely mapped loci, as well as generating useful models of human disease. Furthermore, the utilization of such technology for substitution of mouse loci with their human equivalents could provide unique insights into the expression and regulation of human gene products during development, their communication with other systems, and their involvement in disease induction and progression. An important practical application of such a strategy is the “humanization” of the mouse humoral immune system. Introduction of human immunoglobulin (Ig) loci into mice in which the endogenous Ig polynucleotides and polypeptides have been inactivated offers the opportunity to study the mechanisms underlying programmed expression and assembly of antibodies as well as their role in B cell development. Furthermore, such a strategy could provide an ideal source for the production of fully human monoclonal antibodies (Mabs) an important milestone toward fulfilling the promise of antibody therapy in human disease.

Fully human antibodies are expected to minimize the immunogenic and allergic responses intrinsic to mouse or mouse-derivatized monoclonal antibodies and thus to increase the efficacy and safety of the administered antibodies. The use of fully human antibodies can be expected to provide a substantial advantage in the treatment of chronic and recurring human diseases, such as cancer, which require repeated antibody administrations.

One approach toward this goal was to engineer mouse strains deficient in mouse antibody production to harbor large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human monoclonal antibodies with the desired specificity could be readily produced and selected.

This general strategy was demonstrated in connection with the generation of the first “XenoMouseT” strains as published in 1994. See Green et al., 1994, Nature Genetics, 7:13-21. The XenoMouse strains were engineered with yeast artificial chromosomes (YACS) containing 245 kb and 10 190 kb-sized germline configuration fragments of the human heavy chain locus and kappa light chain locus, respectively, which contained core variable and constant region sequences. Id. The human Ig containing YACs proved to be compatible with the mouse system for both rearrangement and expression of antibodies and were capable of substituting for the inactivated mouse Ig polynucleotides and polypeptides. This was demonstrated by their ability to induce B-cell development, to produce an adult-like human repertoire of fully human antibodies, and to generate antigen-specific human monoclonal antibodies. These results also suggested that introduction of larger portions of the human Ig loci containing greater numbers of V polynucleotides and polypeptides, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through the use of megabase-sized, germline configuration YAC fragments of the human heavy chain loci and kappa light chain loci, respectively, to produce XenoMouse mice. See Mendez et al., 1997, Nature Genetics, 15:146-156; Green and Jakobovits, 1998, J. Exp. Med., 188:483-495; and Green, 1999, Journal of Immunological Methods, 231:11-23, the disclosures of which are hereby incorporated herein by reference.

Human anti-mouse antibody (HAMA) responses have led the industry to prepare chimeric or otherwise humanized antibodies. While chimeric antibodies typically are comprised of a human constant region and a murine variable region, it is expected that certain human anti-chimeric antibody (HACA) responses will be observed, particularly in treatments involving chronic or multi-dose utilizations of the antibody. Thus, it is desirable to provide fully human antibodies against src biomarker polypeptides in order to vitiate concerns and/or effects of HAMA or HACA responses.

Polypeptide antibodies of the invention may be chemically synthesized or produced through the use of recombinant expression systems. Accordingly, the invention further embraces polynucleotides comprising a nucleotide sequence encoding an antibody of the invention and fragments thereof. The invention also encompasses polynucleotides that hybridize under stringent or lower stringency hybridization conditions, e.g., as defined supra, to polynucleotides that encode an antibody, preferably, an antibody that specifically binds to a polypeptide of the invention, preferably, an antibody that binds to a polypeptide having the amino acid sequence of one or more of the src biomarker sequences as set forth in Tables 3-6.

The polynucleotides may be obtained, and the nucleotide sequence of the polynucleotides determined, by any method known in the art. For example, if the nucleotide sequence of the antibody is known, a polynucleotide encoding the antibody can be assembled from chemically synthesized oligonucleotides (e.g., as described in Kutmeier et al., 1994, BioTechniques, 17:242), which, briefly, involves the synthesis of overlapping oligonucleotides containing portions of the sequence encoding the antibody, the annealing and ligating of those oligonucleotides, and then the amplification of the ligated oligonucleotides by PCR.

Alternatively, a polynucleotide encoding an antibody can be generated from nucleic acid from a suitable source. If a clone containing a nucleic acid encoding a particular antibody is not available, but the sequence of the antibody molecule is known, a nucleic acid encoding the immunoglobulin can be chemically synthesized or obtained from a suitable source (e.g., an antibody cDNA library, or a cDNA library generated from, (or a nucleic acid, preferably poly A+ RNA, isolated from), any tissue or cells expressing the antibody, such as hybridoma cells selected to express an antibody of the invention by PCR amplification using synthetic primers hybridizable to the 3′ and 5′ ends of the sequence. Alternatively, cloning using an oligonucleotide probe specific for the particular gene sequence to identify, e.g., a cDNA clone from a cDNA library that encodes the antibody can be employed. Amplified nucleic acids generated by PCR can then be cloned into replicable cloning vectors using any method well known in the art.

Once the nucleotide sequence and corresponding amino acid sequence of the antibody are determined, the nucleotide sequence of the antibody can be manipulated using methods well known in the art for the manipulation of nucleotide sequences, e.g., recombinant DNA techniques, site directed mutapolynucleotides and polypeptidesis, PCR, etc. (see, for example, the techniques described in Sambrook et al., 1990, Molecular Cloning, A Laboratory Manual, 2nd Ed., Cold Spring Harbor Laboratory, Cold Spring Harbor, NY; and Ausubel et al., eds., 1998, Current Protocols in Molecular Biology, John Wiley & Sons, NY, which are both incorporated by reference herein in their entireties), to generate antibodies having a different amino acid sequence, for example, to create amino acid substitutions, deletions, and/or insertions.

In a specific embodiment, the amino acid sequence of the heavy and/or light chain variable domains can be inspected to identify the sequences of the CDRs by methods that are well known in the art, e.g., by comparison to known amino acid sequences of other heavy and light chain variable regions, to determine the regions of sequence hypervariability. Using routine recombinant DNA techniques, one or more of the CDRs can be inserted within framework regions, e.g., into human framework regions, to humanize a non-human antibody, as described supra. The framework regions can be naturally occurring or consensus framework regions, and preferably, are human framework regions (see, e.g., Chothia et al., 1998, J. Mol. Biol., 278:457-479 for a listing of human framework regions).

Preferably, the polynucleotide generated by the combination of the framework regions and CDRs encodes an antibody that specifically binds to a src biomarker polypeptide of the invention. Also preferably, as discussed supra, one or more amino acid substitutions can be made within the framework regions; such amino acid substitutions are performed with the goal of improving binding of the antibody to its antigen. In addition, such methods can be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and are within the skill of the art.

For some uses, such as for in vitro affinity maturation of an antibody of the invention, it is useful to express the V_{H} and V_{L} domains of the heavy and light chains of one or more antibodies of the invention as single chain antibodies, or Fab fragments, in a phage display library using phage display methods as described supra. For example, the cDNAs encoding the V_{H} and V_{L} domains of one or more antibodies of the invention can be expressed in all possible combinations using a phage display library, thereby allowing for the selection of V_{H}/V_{L} combinations that bind to the src biomarker polypeptides according to the present invention with preferred binding characteristics such as improved affinity or improved off rates. In addition, V_{H} and V_{L} segments, particularly, the CDR regions of the V_{H} and V_{L} domains of one or more antibodies of the invention, can be mutated in vitro. Expression of V_{H} and V_{L} domains with “mutant” CDRs in a phage display library allows for the selection of V_{H}/V_{L} combinations that bind to src biomarker proteins which are receptor polypeptides with preferred binding characteristics such as improved affinity or improved off rates.

In phage display methods, functional antibody domains are displayed on the surface of phage particles which carry the polynucleotide sequences encoding them. In particular, DNA sequences encoding the V_{H} and V_{L} domains are amplified from animal cDNA libraries (e.g., human or murine cDNA libraries of lymphoid tissues) or from synthetic cDNA libraries. The DNA encoding the V_{H} and V_{L} domains are joined together by an scFv linker by PCR and cloned into a phagemid vector (e.g., p CANTAB 6 or pComb 3 HSS). The vector is introduced into E. coli via electroporation and the E. coli is infected with helper phage. Phage used in these methods are typically filamentous phage, including fd and M13, and the V_{H} and V_{L} domains are usually recombinantly fused either to the phage gene III or gene VIII. Phage expressing an antigen binding domain that binds to an antigen of interest (i.e., a src biomarker polypeptide or a fragment thereof) can be selected or identified with antigen, e.g., using labeled antigen or antigen bound or captured onto a solid surface or bead.

The antibodies according to the invention can be produced by any method known in the art for the synthesis of antibodies, in particular, by chemical synthesis, by intracellular immunization (i.e., intrabody technology), or preferably, by recombinant expression techniques. Methods of producing antibodies include, but are not limited to, hybridoma technology, EBV transformation, and other methods discussed herein as well as through the use recombinant DNA technology, as discussed below.

Recombinant expression of an antibody of the invention, or fragment, derivative, variant or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule can be produced by recombinant DNA technology using techniques well known in the art. Methods for preparing a protein by expressing a polynucleotide encoding an antibody are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus embraces replicable vectors comprising a nucleotide sequence encoding an antibody molecule of the invention, or a heavy or light chain thereof, or a heavy or light chain variable domain, operably linked to a promoter. Such vectors can include the nucleotide sequence encoding the constant region of the antibody molecule (see, e.g., PCT Publication WO 86/05807; PCT Publication WO 89/01036; and U.S. Pat. No. 5,122,464) and the variable domain of the antibody can be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host expression vector systems can be utilized to express the antibody molecules of the invention. Such expression systems represent vehicles by which the coding sequences of interest can be expressed, their encoded products produced and subsequently purified. These systems also represent cells which can, when transformed or transfected with the appropriate nucleotide coding sequences, express an antibody molecule of the invention in situ. Cell expression systems include, but are not limited, to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces or Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus (CaMV) or tobacco mosaic virus (TMV)), transformed with recombinant plasmid expression vectors (e.g., Ti plasmid) containing antibody coding sequences; or mammalian cell systems (e.g., COS, CHO, BHK, 293, 3T3, NSO cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as E. coli, and more preferably, eukaryotic cells, especially for the expression of whole recombinant antibody molecules, are used for the expression of a recombinant antibody molecule. For example, mammalian cells such as Chinese hamster ovary (CHO) cells, in conjunction with a vector such as the major intermediate early gene promoter element from human cytomegalovirus, is an effective expression system for antibodies (Foecking et al., 1986, Gene, 45:101; Cockett et al., 1990, BioTechnology, 8:2).

In bacterial systems, a number of expression vectors can be advantageously selected depending upon the use intended for the antibody molecule being expressed. For example, when a large quantity of such a protein is to be produced for the generation of pharmaceutical compositions of an antibody molecule, for example, vectors that direct the expression of high levels of fusion protein products that are readily purified are often desirable. Such vectors include, but are not limited to, the E. coli expression vector pUR278 (Ruther et al., 1983, EMBO J., 2:1791), in which the antibody coding sequence can be ligated individually into the vector in-frame with the lacZ coding region so that a fusion protein is produced; pIN vectors (Inouye & Inouye, 1985, Nucleic Acids Res., 13:3101-3109; Van Heeke & Schuster, 1989, J. Biol. Chem., 24:5503-5509; and the like). pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (ACNPV) is used as a vector to express foreign polynucleotides and polypeptides. The virus grows in Spodoptera figuriperda cells. The antibody coding sequence may be cloned individually into non-essential regions (for example the polyhedrin gene) of the virus and placed under control of an AcNPV promoter (for example the polyhedrin promoter).

In mammalian host cells, a number of viral based expression systems can be utilized. In cases in which an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) results in a recombinant virus that is viable and capable of expressing the antibody molecule in infected hosts. (See, e.g., Logan and Shenk, 1984, Proc. Natl. Acad. Sci. USA, 81:355-359). Specific initiation signals can also be required for efficient translation of inserted antibody coding sequences. These signals include the ATG initiation codon and adjacent sequences. Furthermore, the initiation codon must be in-phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see Bittner et al., 1987, Methods in Enzymol., 153:51-544).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein.

Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include, but are not limited to, CHO, VERY, BHK, Hela, COS, MDCK, 293, 3T3, WI38, and in particular, breast cancer cell lines such as, for example, BT483, Hs578T, HTB2, BT20 and T47D, and normal mammary gland cell lines such as, for example, CRL7030 and Hs578Bst.

For long-term, high-yield production of recombinant proteins, stable expression is preferred. For example, cell lines which stably express the antibody molecule can be engineered. Rather than using expression vectors that contain viral origins of replication, host cells can be transformed with DNA controlled by appropriate expression control elements (e.g., promoters, enhancer sequences, transcription terminators, polyadenylation sites, etc.), and a selectable marker. Following the introduction of the foreign DNA, such genetically engineered cells can be allowed to grow for 1-2 days in an enriched medium, and then are typically replated in a selective medium. A selectable marker in the recombinant plasmid confers resistance to the selection and allows cells to stably integrate the plasmid into their chromosomes and grow to form foci which, in turn, can be cloned and expanded into cell lines. This method can advantageously be used to engineer cell lines expressing the antibody molecule. Such engineered cell lines are particularly useful in screening and evaluation of compounds that interact directly or indirectly with the antibody molecule.

A number of selection systems can be used, including but not limited to, herpes simplex virus thymidine kinase (HSV TK), (Wigler et al., 1977, Cell, 11:223), hypoxanthine-guanine phosphoribosyltransferase (HGPRT), (Szybalska and Szybalski, 1992, Proc. Natl. Acad. Sci. USA, 48:202), and adenine phosphoribosyltransferase (Lowy et al., 1980, Cell, 22:817) polynucleotides and polypeptides can be employed in tk-, hgprt-, or aprt-cells (APRT), respectively.

In addition, anti-metabolite resistance can be used as the basis of selection for the following polynucleotides and polypeptides: dhfr, which confers resistance to methotrexate (Wigler et al., 1980, Proc. Natl. Acad. Sci. USA, 77:357; and O'Hare et al., 1981, Proc. Natl. Acad. Sci. USA, 78:1527); gpt, which confers resistance to mycophenolic acid (Mulligan and Berg, 1981, Proc. Natl. Acad. Sci. USA, 78:2072); neo, which confers resistance to the aminoglycoside G418 (Clinical Pharmacy, 12:488-505; Wu and Wu, 1991, Biotherapy, 3:87-95; Tolstoshev, 1993, Ann. Rev. Pharmacol. Toxicol., 32:573-596; Mulligan, 1993, Science, 260:926-932; Anderson, 1993, Ann. Rev. Biochem., 62:191-21; May, 1993, TIB TECH, 11(5):155-215; and hygro, which confers resistance to hygromycin (Santerre et al., 1984, Gene, 30:147). Methods commonly known in the art of recombinant DNA technology can be routinely applied to select the desired recombinant clone, and such methods are described, for example, in Ausubel et al. (eds.), Current Protocols in Molecular Biology, John Wiley & Sons, NY (1993); Kriegler, 1990, Gene Transfer and Expression, A Laboratory Manual, Stockton Press, NY; in Chapters 12 and 13, Dracopoli et al. (eds), Current Protocols in Human Genetics, John Wiley & Sons, NY (1994); Colberre-Garapin et al., 1981. J. Mol. Biol, 150:1, which are incorporated by reference herein in their entireties.

The expression levels of an antibody molecule can be increased by vector amplification (for a review, see Bebbington and Hentschel, The use of vectors based on gene amplification for the expression of cloned polynucleotides and polypeptides in mammalian cells in DNA cloning, Vol. 3. (Academic Press, New York, 1987). When a marker in the vector system expressing an antibody is amplifiable, an increase in the level of inhibitor present in the host cell culture will increase the number of copies of the marker gene. Since the amplified region is associated with the antibody gene, production of the antibody will also increase (Crouse et al., 1983, Mol. Cell. Biol., 3:257).

Vectors which use glutamine synthase (GS) or DHFR as the selectable markers can be amplified in the presence of the drugs methionine sulphoximine or methotrexate, respectively. An advantage of glutamine synthase based vectors are the availability of cell lines (e.g., the murine myeloma cell line, NSO) which are glutamine synthase negative. Glutamine synthase expression systems can also function in glutamine synthase expressing cells (e.g. Chinese Hamster Ovary (CHO) cells) by providing additional inhibitor to prevent the functioning of the endogenous gene.

Vectors that express glutamine synthase as the selectable marker include, but are not limited to, the pEE6 expression vector described in Stephens and Cockett, 1989, Nucl. Acids. Res., 17:7110. A glutamine synthase expression system and components thereof are detailed in PCT publications: W087/04462; W086/05807; W089/01036; W089/10404; and W091/06657 which are incorporated by reference herein in their entireties. In addition, glutamine synthase expression vectors that can be used in accordance with the present invention are commercially available from suppliers, including, for example, Lonza Biologics, Inc. (Portsmouth, NH). The expression and production of monoclonal antibodies using a GS expression system in murine myeloma cells is described in Bebbington et al., 1992, BioTechnology, 10:169 and in Biblia and Robinson, 1995, Biotechnol. Prog., 11:1, which are incorporated by reference herein in their entireties.

A host cell can be co-transfected with two expression vectors of the invention, the first vector encoding a heavy chain derived polypeptide and the second vector encoding a light chain derived polypeptide. The two vectors can contain identical selectable markers which enable equal expression of heavy and light chain polypeptides. Alternatively, a single vector can be used which encodes, and is capable of expressing, both the heavy and light chain polypeptides. In such situations, the light chain should be placed before the heavy chain to avoid an excess of toxic free heavy chain (Proudfoot, 1986, Nature, 322:52; Kohler, 1980, Proc. Natl. Acad. Sci. USA, 77:2197). The coding sequences for the heavy and light chains can comprise cDNA or genomic DNA.

Once an antibody molecule of the invention has been produced by an animal, chemically synthesized, or recombinantly expressed, it can be purified by any method known in the art for the purification of an immunoglobulin or polypeptide molecule, for example, by chromatography (e.g., ion exchange, affinity, particularly by affinity for the specific antigen, Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies that are recombinantly fused or chemically conjugated (including both covalently and non-covalently conjugated) to a polypeptide (or portion thereof, preferably at least 10, 20, 30, 40, 50, 60, 70, 80, 90 or 100 amino acids of the polypeptide) of the present invention to generate fusion proteins. The fusion does not necessarily need to be direct, but can occur through linker sequences. The antibodies can be specific for antigens other than polypeptides (or portions thereof, preferably at least 10, 20, 30, 40, 50, 60, 70, 80, 90 or 100 amino acids of the polypeptide) of the present invention. For example, antibodies can be used to target the polypeptides of the present invention to particular cell types, either in vitro or in vivo, by fusing or conjugating the polypeptides of the present invention to antibodies specific for particular cell surface receptors.

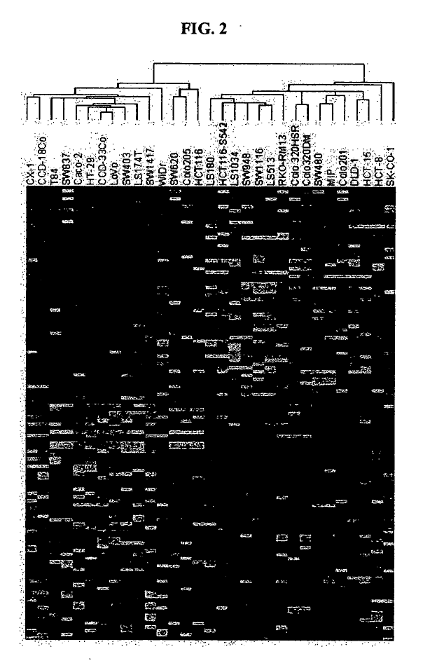

Polypeptides and/or antibodies of the present invention (including fragments or variants thereof) can be fused to either the N-terminal or C-terminal end of the heterologous protein (e.g., immunoglobulin Fc polypeptide or human serum albumin polypeptide). Antibodies of the invention can also be fused to albumin (including, but not limited to, recombinant human serum albumin (see, e.g., U.S. Pat. No. 5,876,969, issued Mar. 2, 1999; EP Patent 0 413 622; and U.S. Pat. No. 5,766,883, issued Jun. 16, 1998, incorporated herein by reference in their entirety), resulting in chimeric polypeptides. In a preferred embodiment, polypeptides and/or antibodies of the present invention (including fragments or variants thereof) are fused with the mature form of human serum albumin (i.e., amino acids 1-585 of human serum albumin as shown in FIGS. 1 and 2 of EP Patent 0 322 094, which is herein incorporated by reference in its entirety). In another preferred embodiment, polypeptides and/or antibodies of the present invention (including fragments or variants thereof) are fused with polypeptide fragments comprising, or alternatively consisting of, amino acid residues 1-z of human serum albumin, where z is an integer from 369 to 419, as described in U.S. Pat. No. 5,766,883 incorporated herein by reference in its entirety.

Polynucleotides encoding src biomarker fusion proteins and antibodies thereto are also encompassed by the invention. Such fusion proteins may, for example, facilitate purification and may increase half-life in vivo. Antibodies fused or conjugated to the polypeptides of the present invention may also be used in in vitro immunoassays and purification methods using methods known in the art. See, e.g., Harbor et al., supra, and PCT publication WO 93/21232; EP 439, 095 ; Naramura et al., 1994, Immunol. Lett., 39:91-99; U.S. Pat. No. 5,474,981; Gillies et al., 1992, Proc. Natl. Acad. Sci. USA, 89:1428-1432; Fell et al., 1991, J. Immunol., 146:2446-2452, which are incorporated by reference herein in their entireties.

The present invention further includes compositions comprising the src biomarker polypeptides of the present invention fused or conjugated to antibody domains other than the variable region domain. For example, the polypeptides of the present invention can be fused or conjugated to an antibody Fc region, or portion thereof. The antibody portion fused to a polypeptide of the present invention can comprise the constant region, hinge region, CH1 domain, CH2 domain, CH3 domain, or any combination of whole domains or portions thereof. The polypeptides can also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. (See, e.g., U.S. Pat. Nos. 5,336,603; 5,622,929; 5,359,046; 5,349,053; 5,447,851; 5,112,946; EP 307,434; EP 367,166; PCT publications WO 96/04388; WO 91/06570; Ashkenazi et al., 1991, Proc. Natl. Acad. Sci. USA, 88:10535-10539; Zheng et al., 1995, J. Immunol., 154:5590-5600; and Vil et al., Proc. Natl. Acad. Sci. USA, 89:11337-11341, which are hereby incorporated by reference herein in their entireties).

As discussed supra, the polypeptides corresponding to a polypeptide, polypeptide fragment, or a variant of one or more of the src biomarker amino acid sequences as set forth in Tables 3-6 can be fused or conjugated to the above antibody portions to increase the in vivo half life of the polypeptides, or for use in immunoassays using methods known in the art. Further, the polypeptides corresponding to one or more of the src biomarker sequences as set forth in Tables 3-6 can be fused or conjugated to the above antibody portions to facilitate purification. For guidance, chimeric proteins having the first two domains of the human CD4 polypeptide and various domains of the constant regions of the heavy or light chains of mammalian immunoglobulins have been described. (EP 394,827; Traunecker et al., 1988, Nature, 331:84-86). The polypeptides of the present invention fused or conjugated to an antibody, or portion thereof, having disulfide-linked dimeric structures (due to the IgG), for example, can also be more efficient in binding and neutralizing other molecules, than the monomeric secreted protein or protein fragment alone. (Fountoulakis et al., 1995, J. Biochem., 270:3958-3964). In many cases, the Fc portion in a fusion protein is beneficial in therapy, diagnosis, and/or screening methods, and thus can result in, for example, improved pharmacokinetic properties. (EP A 232, 262). In drug discovery, for example, human proteins, such as hIL-5, have been fused with Fc portions for the purpose of high-throughput screening assays to identify antagonists of hIL-5. (See, Bennett et al., 1995, J. Molecular Recognition, 8:52-58; and Johanson et al., 1995, J. Biol. Chem., 270:9459-9471). Alternatively, deleting the Fc portion after the fusion protein has been expressed, detected, and purified, may be desired. For example, the Fc portion may hinder therapy and diagnosis if the fusion protein is used as an antigen for immunizations.

Moreover, the antibodies or fragments thereof of the present invention can be fused to marker sequences, such as a peptide, to facilitate their purification. In preferred embodiments, the marker amino acid sequence is a hexa-histidine peptide, such as the tag provided in a pQE vector (QIAGEN, Inc., Chatsworth, Calif), among others, many of which are commercially available. As described in Gentz et al., 1989, Proc. Natl. Acad. Sci. USA, 86:821-824, for instance, hexa histidine provides for convenient purification of the fusion protein. Other peptide tags useful for purification include, but are not limited to, the “HA” tag, which corresponds to an epitope derived from the influenza hemagglutinin (HA) protein (Wilson et al., 1984, Cell, 37:767) and the “flag” tag.

The present invention further encompasses antibodies or fragments thereof conjugated to a diagnostic or therapeutic agent. The antibodies can be used diagnostically to, for example, monitor the development or progression of a tumor as part of a clinical testing procedure, for example, to determine the efficacy of a given treatment regimen. Detection can be facilitated by coupling the antibody to a detectable substance. Nonlimiting examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance can be coupled or conjugated either directly to the antibody (or fragment thereof) or indirectly, through an intermediate (such as, for example, a linker known in the art) using techniques known in the art. (See, for example, U.S. Pat. No. 4,741,900 for metal ions which can be conjugated to antibodies for use as diagnostics according to the present invention).

Nonlimiting examples of suitable enzymes include horseradish peroxidase, alkaline phosphatase, beta-galactosidase, or acetylcholinesterase; Nonlimiting examples of suitable prosthetic group complexes include streptavidin/biotin and avidin/biotin; nonlimiting examples of suitable fluorescent materials include umbelliferone, fluorescein, fluorescein isothiocyanate, rhodamine, dichlorotriazinylamine fluorescein, dansyl chloride or phycoerythrin; a nonlimiting example of a luminescent material includes luminol; nonlimiting examples of bioluminescent materials include luciferase, luciferin, and aequorin; and nonlimiting examples of suitable radioactive material include iodine (¹²⁵I, ¹³¹I), carbon (¹⁴C), sulfur (3sus), tritium (³H), indium (¹¹¹In and other radioactive isotopes of inidium), technetium (⁹⁹Tc, ^{99m}Tc), thallium (20′Ti), gallium (⁶⁸Ga, ⁶⁷Ga), palladium (¹⁰³Pd), molybdenum (⁹⁹Mo), xenon (¹³³Xe), fluorine (¹⁹F), ¹⁵³Sm, ¹⁷⁷Lu, Gd, radioactive Pm, radioactive La, radioactive Yb, ¹⁶⁶Ho, ⁹⁰Y, radioactive Sc, radioactive Re, radioactive Re, ¹⁴²Pr, ¹⁰⁵Rh, and ⁹⁷Ru.

In specific embodiments, the src biomarker polypeptides of the invention are attached to macrocyclic chelators useful for conjugating radiometal ions, including, but not limited to, ¹¹¹In, ¹⁷⁷Lu, ⁹⁰Y, ¹⁶⁶Ho, and ¹⁵³Sm, to polypeptides. In a preferred embodiment, the radiometal ion associated with the macrocyclic chelators attached to the src biomarker polypeptides of the invention is ¹¹¹In. In another preferred embodiment, the radiometal ion associated with the macrocyclic chelator attached to the src biomarker polypeptides. of the invention is ⁹⁰Y. In specific embodiments, the macrocyclic chelator is 1,4,7,10-tetraazacyclododecane-N,N′,N″,N′″-tetraacetic acid (DOTA). In other specific embodiments, the DOTA is attached to the src biomarker polypeptides of the invention via a linker molecule.

Examples of linker molecules useful for conjugating DOTA to a polypeptide are commonly known in the art. (See, for example, DeNardo et al., 1998, Clin. Cancer Res., 4(10):2483-90; Peterson et al., 1999, Bioconjug. Chem., 10(4):553-557; and Zimmerman et al, 1999, Nucl. Med. Biol., 26(8):943-950, which are hereby incorporated by reference in their entirety. In addition, U.S. Pat. Nos. 5,652,361 and 5,756,065, which disclose chelating agents that can be conjugated to antibodies and methods for making and using them, are hereby incorporated by reference in their entireties. Though U.S. Pat. Nos. 5,652,361 and 5,756,065 focus on conjugating chelating agents to antibodies, one skilled in the art can readily adapt the methods disclosed therein in order to conjugate chelating agents to other polypeptides.

Antibodies can also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating therapeutic moieties to antibodies are well known, see, e.g., Arnon et al., “Monoclonal Antibodies For Immunotargeting Of Drugs In Cancer Therapy”, In: Monoclonal Antibodies And Cancer Therapy, Reisfeld et al. (eds.), pp. 243-56, Alan R. Liss, Inc., 1985; Hellstrom et al., “Antibodies For Drug Delivery”, In: Controlled Drug Delivery (2nd Ed.), Robinson et al. (eds.), pp. 623-53, Marcel Deldcer, Inc., 1987; Thorpe, “Antibody Carriers Of Cytotoxic Agents In Cancer Therapy: A Review”, In: Monoclonal Antibodies '84: Biological And Clinical Applications, Pinchera et al. (eds.), pp. 475-506, 1985; “Analysis, Results, And Future Prospective Of The Therapeutic Use Of Radiolabeled Antibody In Cancer Therapy”, In: Monoclonal Antibodies For Cancer Detection And Therapy, Baldwin et al. (eds.), pp. 303-316, Academic Press, 1985; and Thorpe et al., 1982, “The Preparation And Cytotoxic Properties Of Antibody-Toxin Conjugates”, Immunol. Rev., 62:119-158. Alternatively, an antibody can be conjugated to a second antibody to form an antibody heteroconjugate, e.g., as described in U.S. Pat. No. 4,676,980 to Segal, which is incorporated herein by reference in its entirety. An antibody, i.e., an antibody specific for a src biomarker polypeptide of this invention, with or without a therapeutic moiety conjugated to it, and administered alone or in combination with cytotoxic factor(s) and/or cytokine(s), can be used as a therapeutic.

The antibodies of the invention can be utilized for immunophenotyping of cell lines and biological samples. The translation product of the src biomarker-encoding polynucleotides and polypeptides of the present invention can be useful as cell specific marker(s), or more specifically, as cellular marker(s) that are differentially expressed at various stages of differentiation and/or maturation of particular cell types. Monoclonal antibodies directed against a specific epitope, or combination of epitopes, allow for the screening of cellular populations expressing the marker. Various techniques utilizing monoclonal antibodies can be employed to screen for cellular populations expressing the marker(s), including magnetic separation using antibody-coated magnetic beads, “panning” with antibody(ies) attached to a solid matrix (i.e., tissue culture plate), and flow cytometry (See, e.g., U.S. Pat. No. 5,985,660; and Morrison et al., 1999, Cell, 96:737-749).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

Antibodies according to this invention can be assayed for immunospecific binding by any method known in the art. The immunoassays which can be used include, but are not limited to, competitive and non-competitive assay systems using techniques such as BIAcore analysis, FACS (Fluorescence Activated Cell Sorter) analysis, immunofluorescence, immunocytochemistry, Western blots, radioimmunoassays, ELISA (enzyme linked immunosorbent assays), “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays, to name but a few. Such assays are routine and well known and practiced in the art (see, e.g., Ausubel et al, eds, 1994, Current Protocols in Molecular Biology, Vol. 1, John Wiley & Sons, Inc., New York, which is incorporated by reference herein in its entirety). Nonlimiting, exemplary immunoassays are described briefly below.

Immunoprecipitation protocols generally comprise lysing a population of cells in a lysis buffer such as RIPA buffer (i.e., 1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate); adding the antibody of interest to the cell lysate; incubating for a period of time (e.g., 1 to 4 hours) at 4° C.; adding protein A and/or protein G sepharose beads to the cell lysate; incubating for about 60 minutes or more at 4° C.; washing the beads in lysis buffer; and resuspending the beads in SDS/sample buffer. The ability of the antibody of interest to immunoprecipitate a particular antigen can be assessed by, for example, Western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols, see, e.g., Ausubel et al, eds, 1994, Current Protocols in Molecular Biology, Vol. 1, John Wiley & Sons, Inc., New York, at 10.16.1.

Western blot analysis generally comprises preparing protein samples; electrophoresis of the protein samples in a polyacrylamide gel (e.g., 8%-20% SDS PAGE depending on the molecular weight of the antigen); transferring the protein sample from the polyacrylamide gel to a membrane such as nitrocellulose, PVDF or nylon; blocking the membrane in blocking solution (e.g., PBS with 3% BSA or nonfat milk); washing the membrane in washing buffer (e.g., PBS-Tween 20); blocking the membrane with primary antibody (the antibody of interest) diluted in blocking buffer; washing the membrane in washing buffer; blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²p or ¹²⁵I) diluted in blocking buffer; washing the membrane in wash buffer; and detecting the presence of the antigen. One of skill in the art would be knowledgeable as to the parameters that can be modified to increase the signal detected and to reduce the background noise. For further discussion regarding Western blot protocols, see, e.g., Ausubel et al, eds, 1994, Current Protocols in Molecular Biology, Vol. 1, John Wiley & Sons, Inc., New York, at 10.8.1.

ELISAs comprise preparing antigen; coating the wells of a 96 well microtiter plate with antigen; adding to the wells the antibody of interest conjugated to a detectable compound such as an enzymatic substrate (e.g., horseradish peroxidase or alkaline phosphatase); incubating for a period of time; and detecting the presence of the antigen. In ELISAs, the antibody of interest does not have to be conjugated to a detectable compound; instead, a second antibody (which recognizes the antibody of interest) conjugated to a detectable compound can be added to the wells. Further, instead of coating the wells with antigen, the antibody can be first coated onto the well. In this case, a second antibody conjugated to a detectable compound can be added to the antibody-coated wells following the addition of the antigen of interest. One of skill in the art would be knowledgeable as to the parameters that can be modified to increase the signal detected, as well as other variations of ELISAs known in the art. For further discussion regarding ELISAs, see, e.g., Ausubel et al, eds, 1994, Current Protocols in Molecular Biology, Vol. 1, John Wiley & Sons, Inc., New York, at 11.2.1.

The binding affinity of an antibody to an antigen and the off-rate of an antibody-antigen interaction can be determined by competitive binding assays. One example of a competitive binding assay is a radioimmunoassay involving the incubation of labeled antigen (e.g., ³H or ¹²⁵p, or a fragment or variant thereof, with the antibody of interest in the presence of increasing amounts of labeled antigen, and the detection of the antibody bound to the labeled antigen. The affinity of the antibody of interest for a src biomarker protein and the binding off rates can be determined from the data by Scatchard plot analysis. Competition with a second antibody can also be determined using radioimmunoassays. In this case, the src biomarker protein is incubated with antibody of interest conjugated to a labeled compound (e.g., a compound labeled with ³H or ¹²⁵I) in the presence of increasing amounts of an unlabeled second antibody. This kind of competitive assay between two antibodies, may also be used to determine if two antibodies bind to the same or different epitopes.

In a preferred embodiment, BIAcore kinetic analysis is used to determine the binding on and off rates of antibodies (including antibody fragments or variants thereof) to a src biomarker protein, or fragments of a src biomarker protein. Kinetic analysis comprises analyzing the binding and dissociation of antibodies from chips with immobilized src biomarker protein on the chip surface.

It is to be further understood that the above-described techniques for the production, expression, isolation, and manipulation of antibody molecules, for example, by recombinant techniques involving molecular biology, as well as by other techniques related to the analysis of polynucleotides and polypeptides and proteins, are applicable to other polypeptide or peptide molecules of the invention as described herein, in particular, the src biomarker polypeptides or peptides themselves, as applicable or warranted. in accordance with the various embodiments of this invention.

The present invention also embraces a kit for determining or predicting drug susceptibility or resistance by a patient having a disease, particularly a cancer or tumor, preferably, a colon cancer or tumor. Such kits are useful in a clinical setting for use in testing patient's biopsied tumor or cancer samples, for example, to determine or predict if the patient's tumor or cancer will be resistant or sensitive to a given treatment or therapy with a drug, compound, chemotherapy agent, or biological treatment agent. Provided in the kit are the predictor set comprising those polynucleotides and polypeptides correlating with resistance and sensitivity to src or src family tyrosine kinases modulators in a particular biological system, particularly src kinase inhibitors, and preferably comprising a microarray; and, in suitable containers, the modulator compounds for use in testing a cells from patient tissue or patient samples for resistance/sensitivity; and instructions for use. Such kits encompass predictor set comprising those polynucleotides and polypeptides correlating with resistance and sensitivity to modulators of protein tyrosine kinases including members of the Src family of tyrosine kinases, for example, Src, Fgr, Fyn, Yes, Blk, Hck, Lck and Lyn, as well as other protein tyrosine kinases, including, Bcr-abl, Jak, PDGFR, c-kit and Ephr,

Also, as explained above, the kit is not limited to microarrays, but can encompass a variety of methods and systems by which the expression of the predictor/marker polynucleotides and polypeptides can be assayed and/or monitored, both at the level of mRNA and of protein, for example, via PCR assays, e.g., RT-PCR and immunoassay, such as ELISA. In kits for performing PCR, or in situ hybridization, for example, nucleic acid primers or probes from the sequences of one or more of the predictor polynucleotides and polypeptides, such as those described in Tables 3-6 and 13, are supplied, in addition to buffers and reagents as necessary for performing the method, and instructions for use. In kits for performing immunoassays, e.g. ELISAs, immunoblotting assays, and the like, antibodies, or bindable portions thereof, to the src biomarker polypeptides of the invention, or to antigenic or immunogenic peptides thereof, are supplied, in addition to buffers and reagents as necessary for performing the method, and instructions for use.

In another embodiment, the present invention embraces the use of one or more polynucleotides and polypeptides among those of the predictor polynucleotides and polypeptides identified herein that can serve as targets for the development of drug therapies for disease treatment. Such targets may be particularly applicable to treatment of colon disease, such as colon cancers or tumors. Indeed, because these predictor polynucleotides and polypeptides are differently expressed in sensitive and resistant cells, their expression pattern is correlated with relative intrinsic sensitivity of cells to treatment with compounds that interact with and inhibit src tyrosine kinases. Accordingly, the polynucleotides and polypeptides highly expressed in resistant cells may serve as targets for the development of drug therapies for the tumors which are resistant to src tyrosine kinase inhibitor compounds.

### EXAMPLES

The Examples herein are meant to exemplify the various aspects of carrying out the invention and are not intended to limit the scope of the invention in any way. The Examples do not include detailed descriptions for conventional methods employed, such as in the construction of vectors, the insertion of cDNA into such vectors, or the introduction of the resulting vectors into the appropriate host. Such methods are well known to those skilled in the art and are described in numerous publications, for example, Sambrook, Fritsch, and Maniatis, Molecular Cloning: A Laboratory Manual, 2^{nd} Edition, Cold Spring Harbor Laboratory Press, USA, (1989).

### Example 1

### Methods

### IC₅₀ Determination—in Vitro Cytotoxicity Assay

Src tyrosine kinase inhibitor compounds (described in WO 00/62778, published Oct. 26, 2000) were tested for cytotoxicity in vitro against a panel of thirty-one human colon cell lines available from the American Type Culture Collection, ATCC, except CX-1 and MIP, which were obtained from academic investigators. Cytotoxicity was assessed in cells by the MTS (3-(4,5-dimethylthiazol-2-yl)-5-(3-carboxymethoxyphenyl)-2-(4-sulphenyl)-2H-tetrazolium, inner salt) assay (T. L. Riss et al., 1992, Mol. Biol. Cell, 3 (Suppl.):184a).

To carry out the assays, the colon cells were plated at 4,000 cells/well in 96 well microtiter plates and 24 hours later serial diluted drugs were added. The concentration range for the src compounds. used in the cytotoxicity assay was from 5 μg/ml to 0.0016 μg/ml (roughly 10 μM to 0.0032 μM). The cells were incubated at 37° C. for 72 hours at which time the tetrazolium dye, MTS (333 μg/ml final concentration), in combination with the electron coupling agent phenazine methosulfate (25 μM final concentration), was added. A dehydrogenase enzyme in live cells reduces the MTS to a form that absorbs light at 492 nM that can be quantified spectrophotometrically. The greater the absorbency the greater the number of live cells. The results are expressed as an IC₅₀, which is the drug concentration required to inhibit cell proliferation (i.e. absorbance at 450 nM) to 50% of that of untreated control cells. The mean IC₅₀ and standard deviation (SD) from multiple tests for each cell line were calculated.

### Resistant/Sensitive Classification

For each compound, the IC₅₀ for each cell line was log-transformed to log₁₀(IC₅₀), and the log₁₀(IC₅₀) values were then normalized across the 31 colon cell lines. The cell lines with log₁₀(IC₅₀) below the mean log₁₀(IC₅₀) of all 31 cell lines were defined as sensitive to the compound, while those with log₁₀(IC₅₀) above the mean log₁₀(IC₅₀) were considered to be resistant to the compound. The classification of the thirty-one colon cell lines was performed for all four of the src kinase inhibitor compounds. (Table 2). Gene expression profiling

The colon cells were grown to 50-70% confluence, and RNA was isolated using the RNeasyT™ kits (Qiagen, Valencia, Calif.). The quality of the RNA was assessed by measuring the 28s:18s ribosomal RNA ratio by using an Agilent 2100 bioanalyzer (Agilent Technologies, Rockville, Md.). The concentration of total RNA was determined spectrophotometrically. 10 μg of total RNA from each cell line was used to prepare biotinylated probe according to the Affymetrix Manual (Affymetrix Genechip® Technical Manual, 2000). Probes were hybridized to Affymetrix human genome U95Av2 high density oligonucleotide arrays (Affymetrix, Santa Clara, Calif.). The arrays were then washed and stained using the GeneChip® Fluidics station according to the manufacture's instructions (Affymetrix Genechip® Technical Manual, 2000). The HG-U95Av2 array contains approximately 12,000 probe sets which represent approximately 12,000 human gene sequences and ESTs.

### Preprocessing of Microarray Data

Scanned image files were visually inspected for artifacts and analyzed with GeneChip® Expression Analysis software (Affymetrix, Santa Clara, Calif.). The “Absolute Call” (Affymetrix Genechip® Technical Manual, 2000) which is used to determine whether a transcript is detected within one sample, as well as the “Average Difference” (Affymetrix Genechip® Technical Manual, 2000), which serves as a relative indicator of the level of expression of a transcript, were calculated. The hybridization intensity for each sample was scaled to 1,500 (Affymetrix Genechip® Technical Manual, 2000) in order to account for any minor differences in global chip intensity, so that the overall expression level for each cell line was comparable. Affymetrix control sequences were removed prior to analysis.

Of a total of 12,558 represented polynucleotides and polypeptides on the HG-U95Av2 array, 2079 represented polynucleotides and polypeptides were not detected (Absent Call) across all of the thirty-one colon cell lines using the Affymetrix GeneChip® Expression Analysis algorithm; these undetected polynucleotides and polypeptides were excluded from further analysis. The remaining data were transferred to the GeneCluster software (Whitehead Institute; T. R. Golub et al., 1999, Science, 286:531-537). A threshold filter was applied to the gene expression values of the remaining 10,479 represented polynucleotides and polypeptides to remove negative gene expression values and to limit high gene expression values that were not likely to be in the linear range of the Affymetrix fluorescent scanner. The threshold filter converted all gene expression values that were negative, or below 100 units, to 100 units, and all gene expression values that were above 40,000 units to 40,000 units. All represented polynucleotides and polypeptides whose gene expression values were between 100 and 40,000 were not changed.

A second “variation filter” was then applied to the data set to find polynucleotides and polypeptides that were likely to correlate with different properties and features of the panel of thirty-one cell lines. The object of the second filter is to select those polynucleotides and polypeptides whose expression pattern varies across the data set; a gene that does not vary can not provide information about differing properties of the thirty-one cell line panel. For example, if there are two populations of cells within the data set, i.e., fast growing cells and slow growing cells, then a gene whose expression is constant, or whose expression does not change substantially, can not yield information that would correlate to fast or slow cell growth.

The second variation filter was formulated to determine the expression pattern of each gene across the thirty-one cell lines and find polynucleotides and polypeptides that passed the following criteria:

1. The gene must show a three-fold change in absolute expression, i.e., as depicted in the formula:

$$\frac{expression value in any give cell line}{expression value in any other cell line} > 3 or < 0.33$$

2. In addition to 1, the three-fold change must represent an absolute difference of 1000 expression units.

3. In addition, the criteria in #1 and #2 above must be met on three independent occasions within the data set, i.e., Cell line A/B, Cell line E/F and Cell line T/G. (The algorithm does not use a single expression value for one cell line on multiple occasions, i.e., Cell Line A/B, Cell line A/F and Cell line B/F).

The second variation filter reduced the data set to 3008 polynucleotides and polypeptides.

After the second variation filter was applied, each gene was normalized to the mean across all the thirty-one colon cell samples (with the mean set to 0 and standard deviation set to 1) using the following formula:

$$\frac{\begin{matrix}Expression value gene “ Z ” - \\ mean expression value of gene “ Z ” in the 31 cell lines\end{matrix}}{\begin{matrix}Standard deviation of expression \\ value for gene “ Z ” in the 31 cell lines\end{matrix}}$$

This normalized data set was used to select polynucleotides and polypeptides which significantly correlated with the property of sensitivity toward a drug class as described herein.

### Example 2

### PCr Expression Profiling

RNA quantification is performed using the Taqman® real-time-PCR fluorogenic assay. The Taqman® assay is one of the most precise methods for assaying the concentration of nucleic acid templates.

All cell lines are grown using standard cell culture conditions: RPMI 1640 supplemented to contain 10% fetal bovine serum, 100 IU/ml penicillin, 100 mg/ml streptomycin, 2 mM L-glutamine and 10 mM Hepes (all from GibcoBRL, Rockville, Md.). Eighty percent confluent cells are washed twice with phosphate-buffered saline (GibcoBRL) and harvested using 0.25% trypsin (GibcoBRL). RNA is prepared using standard methods, preferably, employing the RNeasy Kit commercially available from Qiagen (Valencia, Calif.).

cDNA template for real-time PCR can be generated using the Superscript™ First Strand Synthesis system for RT-PCR. Representative forward and reverse RT-PCT primers for each of the src biomarker polynucleotides and polypeptides of the present invention are provided in Tables 13 herein.

SYBR Green real-time PCR reactions are prepared as follows: The reaction mix contains 20 ng first strand cDNA; 50 nM Forward Primer (one or more primers selected from SEQ ID NO:391 to 591); 50 nM Reverse Primer (one or more primers selected from SEQ ID NO:592 to 792); 0.75×SYBR Green I (Sigma); 1×SYBR Green PCR Buffer (50 mMTris-HCl pH 8.3, 75 mM KCl); 10% DMSO; 3 mM MgCl₂; 300 μM each dATP, dGTP, dTTP, dCTP; 1 U Platinum® Taq DNA Polymerase High Fidelity (Cat# 11304-029; Life Technologies; Rockville, Md.), 1:50 dilution; ROX (Life Technologies).

Real-time PCR is performed using an Applied Biosystems 5700 Sequence Detection System. Conditions are 95° C. for 10 minutes (denaturation and activation of Platinum® Taq DNA Polymerase), 40 cycles of PCR (95° C. for 15 seconds, 60° C. for 1 minute). PCR products are analyzed for uniform melting using an analysis algorithm built into the 5700 Sequence Detection System.

cDNA quantification used in the normalization of template quantity is performed using Taqman® technology. Taqman® reactions are prepared as follows: The reaction mix comprises 20 ng first strand cDNA; 25 nM GAPDH-F3, Forward Primer; 250 nM GAPDH-R1 Reverse Primer; 200 nM GAPDH-PVIC Taqman® Probe (fluorescent dye labeled oligonucleotide primer); 1× Buffer A (Applied Biosystems); 5.5 mM MgCl₂; 300 μM dATP, dGTP, dTTP, dCTP; 1 U Amplitaq Gold (Applied Biosystems). GAPDH, D-glyceraldehyde-3-phosphate dehydrogenase, is used as a control to normalize mRNA levels.

Real-time PCR is performed using an Applied Biosystems 7700 Sequence Detection System. Conditions are 95° C. for 10 minutes (denaturation and activation of Amplitaq Gold), 40 cycles of PCR (95° C. for 15 seconds, 60° C. for 1 minute).

The sequences for the GAPDH oligonucleotides used in the Taqman® reactions are as follows:

| | |
|---|---|
| GAPDH-F3: | |
| 5′-AGCCGAGCCACATCGCT-3′ | (SEQ ID NO:793) |
| GAPDH-R1: | |
| 5′-GTGACCAGGCGCCCAATAC-3′ | (SEQ ID NO:794) |
| GAPDH-PVIC Taqman ® Probe-VIC- | |
| 5′-CAAATCCGTTGACTCCGACCTTCACCTT-3′ | (SEQ ID NO:795) |
| TAMRA. | |

The Sequence Detection System generates a Ct (threshold cycle) value that is used to calculate a concentration for each input cDNA template. cDNA levels for each gene of interest are normalized to GAPDH cDNA levels to compensate for variations in total cDNA quantity in the input sample. This is done by generating GAPDH Ct values for each cell line. Ct values for the gene of interest and GAPDH are inserted into a modified version of the δδCt equation (Applied Biosystems Prism® 7700 Sequence Detection System User Bulletin #2), which is used to calculate a GAPDH normalized relative cDNA level for each specific cDNA. The δδCt equation is as follows: relative quantity of nucleic acid template=2^{δδCt}=2^{(δCta-δCtb)}, where δCta=Ct target−Ct GAPDH, and δCtb=Ct reference−Ct GAPDH. (No reference cell line is used for the calculation of relative quantity; δCtb is defined as 21).

### Example 3

### Production of an Antibody Directed Against Src Biomarker Polypeptides

Anti-src biomarker polypeptide antibodies of the present invention can be prepared by a variety of methods. As one example of an antibody-production method, cells expressing a polypeptide of the present invention are administered to an animal to induce the production of sera containing polyclonal antibodies directed to the expressed polypeptides. In a preferred method, the expressed protein is prepared, preferably isolated and purified, to render it substantially free of natural contaminants, using techniques commonly practiced in the art. Such a preparation is then introduced into an animal in order to produce polyclonal antisera of greater specific activity for the expressed and isolated polypeptide.

In a most preferred method, the antibodies of the present invention are monoclonal antibodies (or protein binding fragments thereof) and can be prepared using hybridoma technology as detailed hereinabove. Cells expressing the polypeptide can be cultured in any suitable tissue culture medium; however, it is preferable to culture cells in Earle's modified Eagle's medium supplemented to contain 10% fetal bovine serum (inactivated at about 56° C.), and supplemented to contain about 10 g/l nonessential amino acids, about 1,000 U/ml penicillin, and about 100 μg/ml streptomycin.

The splenocytes of immunized (and boosted) mice are extracted and fused with a suitable myeloma cell line. Any suitable myeloma cell line can be employed in accordance with the present invention; however, it is preferable to employ the parent myeloma cell line (SP2/0), available from the ATCC. After fusion, the resulting hybridoma cells are selectively maintained in HAT medium, and then cloned by limiting dilution as described by Wands et al. (1981, Gastroenterology, 80:225-232). The hybridoma cells obtained through such a selection are then assayed to identify those cell clones that secrete antibodies capable of binding to the polypeptide immunogen, or a portion thereof.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an immunized animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce the formation of further protein-specific antibodies.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known and practiced in the art. (See, e.g., for review, Morrison, 1985, Science, 229:1202); Oi et al., 1986, BioTechniques, 4:214; Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., 1984, Nature, 312:643; and Neuberger et al., 1985, Nature, 314:268).

### Example 4

### Immunofluorescence Assats

The following immunofluorescence protocol may be used, for example, to verify src biomarker protein expression on cells, or, for example, to check for the presence of one or more antibodies that bind src biomarker protein expressed on the surface of cells. Briefly, Lab-Tek II chamber slides are coated overnight at 4° C. with 10 micrograms/milliliter (μg/ml) of bovine collagen Type II in DPBS containing calcium and magnesium (DPBS++). The slides are then washed twice with cold DPBS++ and seeded with 8000 CHO-CCR5 or CHO pC4 transfected cells in a total volume of 125 μl and incubated at 37° C. in the presence of 95% oxygen/5% carbon dioxide.

The culture medium is gently removed by aspiration and the adherent cells are washed twice with DPBS++ at ambient temperature. The slides are blocked with DPBS++ containing 0.2% BSA (blocker) at 0-4° C. for one hour. The blocking solution is gently removed by aspiration, and 125 μl of antibody containing solution (an antibody containing solution may be, for example, a hybridoma culture supernatant which is usually used undiluted, or serum/plasma which is usually diluted, e.g., a dilution of about 1/100 dilution). The slides are incubated for 1 hour at 0-4° C. Antibody solutions are then gently removed by aspiration and the cells are washed 5 times with 400 μl of ice cold blocking solution. Next, 125 μl of 1 μg/ml rhodamine labeled secondary antibody (e.g., anti-human IgG) in blocker solution is added to the cells. Again, cells are incubated for 1 hour at 0-4° C.

The secondary antibody solution is then gently removed by aspiration and the cells are washed 3 times with 400 μl of ice cold blocking solution, and 5 times with cold DPBS++. The cells are then fixed with 125 μl of 3.7% formaldehyde in DPBS++ for 15 minutes at ambient temperature. Thereafter, the cells are washed 5 times with 400 μl of DPBS++ at ambient temperature. Finally, the cells are mounted in 50% aqueous glycerol and viewed in a fluorescence microscope using rhodamine filters.

### BRIEF DESCRIPTION OF THE SEQUENCE LISTING

Incorporated herein by reference in its entirety is a Sequence Listing, including SEQ ID NO:1 through SEQ ID NO:390, which include nucleic acid and amino acid sequences of the src biomarkers as presented in Tables 3-6 herein. The Sequence Listing also contains representative primer pairs that may be used in RT-PCR assays for any of the predictor polynucleotides and polypeptides of the present invention, including SEQ ID NO:391 through SEQ ID NO:792. The Sequence Listing is contained on a compact disc, i.e., CD-ROM, three identical copies of which are filed herewith. The Sequence Listing, in IBM/PC MS-DOS format (named “D0185.np.ST25.txt”), PatentIn Version 3.2, was recorded on 17 Jan., 2003, and is 2007 kilobytes in size.

TABLE 1

| | Mean IC50 | | Mean IC50 | | Mean IC50 | | Mean IC50 | |
|---|---|---|---|---|---|---|---|---|
| Cell Lines | (uM) | +/−SD | (uM) | +/−SD | (uM) | +/−SD | (uM) | +/−SD |
| WiDr | 0.041 | 0.015 | 0.007 | 0.004 | 0.185 | 0.096 | 0.040 | 0.010 |
| HT-29 | 0.042 | 0.023 | 0.038 | 0.023 | 0.489 | 0.204 | 0.082 | 0.044 |
| LoVo | 0.055 | 0.041 | 0.005 | 0.002 | 1.723 | 1.496 | 0.028 | 0.016 |
| HCT-15 | 0.153 | 0.066 | 0.326 | 0.095 | 3.409 | 1.418 | 0.221 | 0.029 |
| CCD-18Co | 0.215 | 0.247 | 0.028 | 0.043 | 1.955 | 1.611 | 0.099 | 0.111 |
| Caco-2 | 0.233 | 0.099 | 0.538 | 0.344 | 0.429 | 0.108 | 0.295 | 0.074 |
| CCD-33Co | 0.311 | 0.221 | 0.124 | 0.178 | 1.068 | 0.745 | 0.241 | 0.079 |
| LS174T | 0.505 | 0.199 | 0.060 | 0.039 | 2.279 | 1.354 | 0.356 | 0.191 |
| SW1417 | 0.505 | 0.476 | 0.021 | 0.028 | 0.187 | 0.246 | 0.096 | 0.081 |
| SW837 | 3.318 | 1.276 | 3.985 | 3.055 | 0.460 | 0.263 | 2.110 | 1.588 |
| DLD-1 | 4.506 | 1.002 | 2.121 | 1.613 | 6.272 | 3.415 | 2.545 | 1.709 |
| RKO-RM13 | 4.686 | 1.917 | 2.761 | 0.454 | 3.296 | 0.749 | 5.552 | 1.647 |
| MIP | 6.548 | 2.311 | 5.822 | 1.294 | 10.565 | 1.557 | 6.097 | 3.398 |
| CX-1 | 7.801 | 3.677 | 7.925 | 3.886 | 2.517 | 1.225 | 7.691 | 1.287 |
| HCT116-S542 | 8.726 | 2.109 | 8.353 | 0.286 | 11.261 | 0.000 | 7.799 | 3.225 |
| SK-CO-1 | 9.533 | 0.398 | 9.912 | 0.000 | 9.038 | 4.446 | 8.900 | 0.000 |
| Colo201 | 9.814 | 0.000 | 7.008 | 1.500 | 7.120 | 1.755 | 6.972 | 2.832 |
| Colo205 | 9.814 | 0.000 | 7.952 | 2.414 | 4.374 | 1.613 | 4.891 | 2.474 |
| Colo320DM | 9.814 | 0.000 | 9.912 | 0.000 | 11.261 | 0.000 | 8.926 | 0.000 |
| HCT116 | 9.814 | 0.000 | 7.903 | 2.422 | 11.261 | 0.000 | 8.926 | 0.000 |
| HCT-8 | 9.814 | 0.000 | 5.362 | 3.142 | 7.743 | 3.991 | 7.312 | 1.974 |
| SW403 | 9.814 | 0.000 | 0.123 | 0.171 | 0.206 | 0.185 | 7.868 | 1.937 |
| SW480 | 9.814 | 0.000 | 7.286 | 2.893 | 11.261 | 0.000 | 8.926 | 0.000 |
| SW620 | 9.814 | 0.000 | 7.622 | 0.827 | 11.261 | 0.000 | 8.926 | 0.000 |
| T84 | 9.814 | 0.000 | 0.682 | 0.492 | 0.715 | 0.238 | 4.994 | 3.406 |
| Colo 320HSR | 10.571 | 0.000 | 10.684 | 0.000 | 11.261 | 0.000 | 9.524 | 0.000 |
| LS1034 | 10.571 | 0.000 | 10.684 | 0.000 | 11.261 | 0.000 | 9.524 | 0.000 |
| LS180 | 10.571 | 0.000 | 1.890 | 0.620 | 11.261 | 0.000 | 2.558 | 0.544 |
| LS513 | 10.571 | 0.000 | 2.254 | 1.283 | 11.261 | 0.000 | 9.524 | 0.000 |
| SW1116 | 10.571 | 0.000 | 1.831 | 0.923 | 11.261 | 0.000 | 9.524 | 0.000 |
| SW948 | 10.571 | 0.000 | 1.756 | 1.007 | 11.261 | 0.000 | 9.524 | 0.000 |

TABLE 2

| Cell Lines | BMS-A | BMS-B | BMS-C | BMS-D |
|---|---|---|---|---|
| WiDr | S | S | S | S |
| HT-29 | S | S | S | S |
| Lo Vo | S | S | S | S |
| CCD-18Co | S | S | S | S |
| Caco-2 | S | S | S | S |
| CCD-33Co | S | S | S | S |
| LS174T | S | S | S | S |
| SW1417 | S | S | S | S |
| HCT-15 | S | S | R | S |
| T84 | R | S | S | R |
| SW403 | R | S | S | R |
| SW837 | R | R | S | R |
| CX-1 | R | R | S | R |
| DLD-1 | R | R | R | R |
| RKO-RM13 | R | R | R | R |
| MIP | R | R | R | R |
| HCT116-S542 | R | R | R | R |
| SK-CO-1 | R | R | R | R |
| Colo201 | R | R | R | R |
| Colo205 | R | R | R | R |
| Colo320DM | R | R | R | R |
| HCT116 | R | R | R | R |
| HCT-8 | R | R | R | R |
| SW480 | R | R | R | R |
| SW620 | R | R | R | R |
| Colo 320HSR | R | R | R | R |
| LS1034 | R | R | R | R |
| LS180 | R | R | R | R |
| LS513 | R | R | R | R |
| SW1116 | R | R | R | R |
| SW948 | R | R | R | R |

TABLE 3

| | | Common to all 4 BMS | | | | | | |
|---|---|---|---|---|---|---|---|---|
| | Highly | compounds | | | | | | SEQ ID |
| | Expressed | (BMS-A, BMS- | Common to | Common to | | | SEQ ID | NO: of |
| | Cells (Sensitive | B, BMS-C, | BMS-B | BMS-C | Genbank | | NO: of | Amino |
| Gene # | or Resistent) | BMS-D) | Compound | Compound | Accession # | UniGene Title | DNA | Acid |
| 1 | Sensitive cells | yes | | | AB014558 | cryptochrome 2 (photolyase-like) | 1 | 202 |
| 2 | Sensitive cells | yes | | | NM_006979 | HLA class II region expressed gene | 2 | 203 |
| | | | | | | KE4 | | |
| 3 | Sensitive cells | yes | | | M22489 | bone morphogenetic protein 2 | 3 | 204 |
| 4 | Sensitive cells | yes | | | AB023194 | KIAA0977 protein | 4 | 205 |
| 5 | Sensitive cells | yes | | | U03688 | cytochrome P450, subfamily I | 5 | 206 |
| | | | | | | (dioxin-inducible), polypeptide 1 | | |
| | | | | | | (glaucoma 3, primary infantile) | | |
| 6 | Sensitive cells | yes | | | M88458 | kDEL (Lys-Asp-Glu-Leu) | 6 | 207 |
| | | | | | | endoplasmic reticulum protein | | |
| | | | | | | retention receptor 2 | | |
| 7 | Sensitive cells | yes | | | L13463 | regulator of G-protein signalling 2, | 7 | 208 |
| | | | | | | 24 kD | | |
| 8 | Sensitive cells | | yes | | U21551 | branched chain aminotransferase 1, | 8 | 209 |
| | | | | | | cytosolic | | |
| 9 | Sensitive cells | yes | | | AF000560 | Homo sapiens TTF-I interacting | 9 | 210 |
| | | | | | | peptide 20 mRNA, partial cds | | |
| 10 | Sensitive cells | | | | AF102265 | N-acetylglucosamine-phosphate | 10 | 211 |
| | | | | | | mutase | | |
| 11 | Sensitive cells | | | yes | X06272 | signal recognition particle receptor | 11 | 212 |
| | | | | | | (‘docking protein’) | | |
| 12 | Sensitive cells | yes | | | L40802 | hydroxysteriod (17-beta) | 12 | 213 |
| | | | | | | dehydrogenase 2 | | |
| 13 | Sensitive cells | yes | | | X13916 | low density lipoprotein-related | 13 | 214 |
| | | | | | | protein 1 (alpha-2-macroglobulin | | |
| | | | | | | receptor) | | |
| 14 | Sensitive cells | yes | | | AF009674 | axin | 14 | 215 |
| 15 | Sensitive cells | yes | | | M73077 | glucocorticoid receptor DNA | 15 | 216 |
| | | | | | | binding factor 1 | | |
| 16 | Sensitive cells | yes | | | U15655 | Ets2 repressor factor | 16 | 217 |
| 17 | Sensitive cells | | | | AB014520 | KIAA0620 protein | 17 | 218 |
| 18 | Sensitive cells | yes | | | M58603 | nuclear factor of kappa light | 18 | 219 |
| | | | | | | polypeptide gene enhancer in B- | | |
| | | | | | | cells 1 (p105) | | |
| 19 | Sensitive cells | | | | X76104 | death-associated protein kinase 1 | 19 | 220 |
| 20 | Sensitive cells | yes | | | AI659108 | Homo sapiens, clone | 20 | N/A |
| | | | | | | IMAGE: 3908182, mRNA, partial | | |
| | | | | | | cds | | |
| 21 | Sensitive cells | yes | | | U72649 | BTG family, member 2 | 21 | 221 |
| 22 | Sensitive cells | | yes | | M64571 | microtubule-associated protein 4 | 22 | 222 |
| 23 | Sensitive cells | yes | | | X77909 | nuclear factor of kappa light | 23 | 223 |
| | | | | | | polypeptide gen enhancer in B- | | |
| | | | | | | cells inhibitor-like 1 | | |
| 24 | Sensitive cells | | | | M34064 | cadherin 2, type 1, N-cadherin | 24 | 224 |
| | | | | | | (neuronal) | | |
| 25 | Sensitive cells | | | | AL050345 | chromosome 22 open reading frame 2 | 25 | 225 |
| 26 | Sensitive cells | yes | | | AB006622 | KIAA0284 protein | 26 | 226 |
| 27 | Sensitive cells | yes | | | AB029027 | KIAA1104 protein | 27 | 227 |
| 28 | Sensitive cells | yes | | | U51903 | IQ motif containing GTPase | 28 | 228 |
| | | | | | | activating protein 2 | | |
| 29 | Sensitive cells | | | | AF041259 | zinc finger protein 217 | 29 | 229 |
| 30 | Sensitive cells | yes | | | AB026891 | solute carrier family 7, (cationic | 30 | 230 |
| | | | | | | amino acid transporter, y+ system) | | |
| | | | | | | member 11 | | |
| 31 | Sensitive cells | | | | AB007960 | SH3-domain, GRB2-like, | 31 | 231 |
| | | | | | | endophilin B1 | | |
| 32 | Sensitive cells | | | | D63390 | platelet-activating factor | 32 | 232 |
| | | | | | | acetylhydrolase, isoform Ib, beta | | |
| | | | | | | subunit (30 kD) | | |
| 33 | Sensitive cells | | | | L10678 | profilin 2 | 33 | 233 |
| 34 | Sensitive cells | yes | | | X60708 | dipeptidylpeptidase IV (CD26, | 34 | 234 |
| | | | | | | adenosine deaminase complexing | | |
| | | | | | | protein 2) | | |
| 35 | Sensitive cells | | | | Y15521 | acetylserotonin O- | 35 | 235 |
| | | | | | | methyltransferase-like | 35 | 235 |
| 36 | Sensitive cells | | yes | | AI038821 | v-Ha-ras Harvey rat sarcoma viral | 36 | 236 |
| | | | | | | oncogene homolog | | |
| 37 | Sensitive cells | | | | X84740 | ligase III, DNA, ATP-dependent | 37 | 237 |
| 38 | Sensitive cells | | | | M23115 | ATPase, Ca++ transporting, cardiac | 38 | 238 |
| | | | | | | muscle, slow twitch 2 | | |
| 39 | Sensitive cells | | | | NM017432 | prostate tumor over expressed gene 1 | 39 | 239 |
| 40 | Sensitive cells | | | | Y12781 | (transducin (beta)-like 1 | 40 | 240 |
| 41 | Sensitive cells | yes | | | K03498 | Homo sapiens endogenous | 41 | 241 |
| | | | | | | retrovirus HER V-K104 long | | |
| | | | | | | terminal repeat, complete sequence; | | |
| | | | | | | and Gag protein (gag) and envelope | | |
| | | | | | | protein (env) polynucleotides and | | |
| | | | | | | polypeptides, complete cds | | |
| 42 | Sensitive cells | | | | AF030335 | purinergic receptor P2Y, G-protein | 42 | 242 |
| | | | | | | coupled, 11 | | |
| 43 | Sensitive cells | | | | X93209 | nardilysin (N-arginine dibasic | 43 | 243 |
| | | | | | | convertase) | | |
| 44 | Sensitive cells | | | | AF068744 | double homeobox, 2 | 44 | 244 |
| 45 | Sensitive cells | | | | AF072247 | methyl-CpG binding domain | 45 | 245 |
| | | | | | | protein 3 | | |
| 46 | Sensitive cells | | yes | | U41344 | proline arginine-rich end leucine- | 46 | 246 |
| | | | | | | rich repeat protein | | |
| 47 | Sensitive cells | yes | | | D13413 | heterogeneous nuclear | 47 | 247 |
| | | | | | | ribonucleoprotein U (scaffold | | |
| | | | | | | attachment factor A) | | |
| 48 | Sensitive cells | yes | | | M69023 | tetraspan 3 | 48 | 248 |
| 49 | Sensitive cells | | | | J04599 | biglycan | 49 | 249 |
| 50 | Sensitive cells | | | | U79267 | protein phosphatase 4, regulatory | 50 | 250 |
| | | | | | | subunit 1 | | |
| 51 | Sensitive cells | yes | | | AF155654 | Human putative ribosomal protein | 51 | 251 |
| | | | | | | S1 mRNA | | |
| 52 | Sensitive cells | | | | X12794 | nuclear receptor subfamily 2, group | 52 | 252 |
| | | | | | | F, member 6 | | |
| 53 | Sensitive cells | | | | U51166 | thymine-DNA glycosylase | 53 | 253 |
| 54 | Sensitive cells | yes | | | L07261 | adducin 1 (alpha) | 54 | 254 |
| 55 | Sensitive cells | | | | U97188 | IGF-II mRNA-binding protein 3 | 55 | 255 |
| 56 | Sensitive cells | yes | | | L37033 | FK506-binding protein 8 (38 kD) | 56 | 256 |
| 57 | Sensitive cells | yes | | | Y09846 | SHC (Src homology 2 domain- | 57 | 257 |
| | | | | | | containing) transforming protein 1 | | |
| 58 | Sensitive cells | yes | | | AF093420 | hsp70-interacting protein | 58 | 258 |
| 59 | Sensitive cells | yes | | | U19775 | nitrogen-activated protein kinase 14 | 59 | 259 |
| 60 | Sensitive cells | | | | J04027 | ATPase, Ca++ transporting, plasma | 60 | 260 |
| | | | | | | membrane 1 | | |
| 61 | Resistant cells | yes | | | Y18483 | solute carrier family 7 (cationic | 61 | 261 |
| | | | | | | amino acid transporter, y+ system), | | |
| | | | | | | member 8 | | |
| 62 | Resistant cells | yes | | | U57352 | amiloride-sensitive cation channel | 62 | 262 |
| | | | | | | 1, neuronal (degenerin) | | |
| 63 | Resistant cells | yes | | | U34994 | protein kinase, DNA-activated, | 63 | 263 |
| | | | | | | catalytic polypeptide | | |
| 64 | Resistant cells | yes | | | X79067 | zinc finger protein 36, C3H type- | 64 | 264 |
| | | | | | | like 1 | | |
| 65 | Resistant cells | yes | | | AB011535 | FAT tumor suppressor (Drosophila) | 65 | 265 |
| | | | | | | homolog 2 | | |
| 66 | Resistant cells | yes | | | U90902 | Human clone 23612 mRNA | 66 | N/A |
| | | | | | | sequence | | |
| 67 | Resistant cells | | | | AB009282 | cytochrome b5 outer mitochondrial | 67 | 266 |
| | | | | | | membrane precursor | | |
| 68 | Resistant cells | | yes | | AJ001685 | killer cell lectin-like receptor | 68 | 267 |
| | | | | | | subfamily C, member 3 | | |
| 69 | Resistant cells | yes | | | S37730 | insulin-like growth factor binding | 69 | 268 |
| | | | | | | protein 2 (36 kD) | | |
| 70 | Resistant cells | | yes | | U37518 | tumor necrosis factor (ligand) | 70 | 269 |
| | | | | | | superfamily, member 10 | | |
| 71 | Resistant cells | yes | | | AC005329 | NADH dehydrogenase (ubiquinone) | 71 | 270 |
| | | | | | | Fe—S protein 7 (20 kD) (NADH- | | |
| | | | | | | coenzyme Q reductase) | | |
| 72 | Resistant cells | | yes | | AB009426 | apolipoprotein B mRNA editing | 72 | 271 |
| | | | | | | enzyme, catalytic polypeptide 1 | | |
| 73 | Resistant cells | | yes | | X70340 | transforming growth factor, alpha | 73 | 272 |
| 74 | Resistant cells | yes | | | U81561 | protein tyrosine phosphatase, | 74 | 273 |
| | | | | | | receptor type, N polypeptide 2 | | |
| 75 | Resistant cells | | yes | | X70040 | macrophage stimulating 1 receptor | 75 | 274 |
| | | | | | | (c-met-related tyrosine kinase) | | |
| 76 | Resistant cells | yes | | | AB000449 | vaccinia related kinase 1 | 76 | 275 |
| 77 | Resistant cells | yes | | | D87119 | GS3955 protein | 77 | 276 |
| 78 | Resistant cells | yes | | | X06745 | polymerase (DNA directed), alpha | 78 | 277 |
| 79 | Resistant cells | yes | | | X78817 | Rho GTPase activating protein 4 | 79 | 278 |
| 80 | Resistant cells | yes | | | AF070530 | hypothetical protein, clone 24751 | 80 | 279 |
| 81 | Resistant cells | yes | | | L43821 | enhancer of filamentation 1 (cas- | 81 | 280 |
| | | | | | | like docking; Crk-associated | | |
| | | | | | | substrate related) | | |
| 82 | Resistant cells | yes | | | AF007156 | KIAA0751 gene product | 82 | 281 |
| 83 | Resistant cells | yes | | | AB014566 | KIAA0666 protein | 83 | 282 |
| 84 | Resistant cells | | | yes | U71364 | serine (or cysteine) proteinase | 84 | 283 |
| | | | | | | inhibitor, clade B (ovalbumin), | | |
| | | | | | | member 9 | | |
| 85 | Resistant cells | | | | U93305 | Homo sapiens A4 differentiation- | 85 | 284 |
| | | | | | | dependent protein (A4), triple LIM | | |
| | | | | | | domain protein (LMO6), and | | |
| | | | | | | synaptophysin (SYP) | | |
| | | | | | | polynucleotides and polypeptides, | | |
| | | | | | | complete cds; and calcium channel | | |
| | | | | | | alpha-1 subunit (CACNA1F) gene, | | |
| | | | | | | partial cds | | |
| 86 | Resistant cells | yes | | | AB006626 | histone deacetylase 4 | 86 | 285 |
| 87 | Resistant cells | yes | | | M31682 | inhibin, beta B (activin AB beta | 87 | 286 |
| | | | | | | polypeptide) | | |
| 88 | Resistant cells | yes | | | AF031824 | cystatin F (leukocystatin) | 88 | 287 |
| 89 | Resistant cells | | yes | | AF035299 | docking protein 1, 62 kD | 89 | 288 |
| | | | | | | (downstream of tyrosine kinase 1) | | |
| 90 | Resistant cells | | | | X82207 | ARP1 (actin-related protein 1, | 90 | 289 |
| | | | | | | yeast) homolog B (centractin beta) | | |
| 91 | Resistant cells | yes | | | U84570 | chromosome 21 open reading frame 2 | 91 | 290 |
| 92 | Resistant cells | | | | AA873266 | pyruvate dehydrogenase kinase, | 92 | 291 |
| | | | | | | isoenzyme 3 | | |
| 93 | Resistant cells | yes | | | X90976 | runt-related transcription factor 1 | 93 | 292 |
| | | | | | | (acute myeloid leukemia 1; aml 1 | | |
| | | | | | | oncogene) | | |
| 94 | Resistant cells | yes | | | D89377 | msh (Drosophila) homeo box | 94 | 293 |
| | | | | | | homolog 2 | | |
| 95 | Resistant cells | | | | M57730 | ephrin-A1 | 95 | 294 |
| 96 | Resistant cells | yes | | | U68111 | protein phosphatase 1, regulatory | 96 | 295 |
| | | | | | | (inhibitor) subunit 2 | | |
| 97 | Resistant cells | yes | | | L07540 | replication factor C (activator 1) 5 | 97 | 296 |
| | | | | | | (36.5 kD) | | |
| 98 | Resistant cells | | | | M65066 | protein kinase, cAMP-dependent, | 98 | 297 |
| | | | | | | regulatory, type I, beta | | |
| 99 | Resistant cells | yes | | | M34182 | protein kinase, cAMP-dependent, | 99 | 298 |
| | | | | | | catalytic, gamma | | |
| 100 | Resistant cells | yes | | | L34059 | cadherin 4, type 1, R-cadherin | 100 | 299 |
| | | | | | | (retinal) | | |
| 101 | Resistant cells | | | | L25665 | guanine nucleotide binding protein- | 101 | 300 |
| | | | | | | like 1 | | |
| 102 | Resistant cells | yes | | | AL050290 | spermidine/spermine N1- | 102 | 301 |
| | | | | | | acetyltransferase | | |
| 103 | Resistant cells | | | | X67325 | interferon, alpha-inducible protein 27 | 103 | 302 |
| 104 | Resistant cells | yes | | | AA595596 | ADP-ribosyltransferase (NAD+; | 104 | N/A |
| | | | | | | poly(ADP-ribose) polymerase)-like 2 | | |
| 105 | Resistant cells | yes | | | AF003837 | jagged 1 (Alagille syndrome) | 105 | 303 |
| 106 | Resistant cells | yes | | | M87339 | replication factor C (activator 1) 4 | 106 | 304 |
| | | | | | | (37 kD) | | |
| 107 | Resistant cells | | | | A1813532 | tumor necrosis factor receptor | 107 | N/A |
| | | | | | | superfamily, member 1B | | |
| 108 | Resistant cells | yes | | | AB018306 | KIAA0763 gene product | 108 | 305 |
| 109 | Resistant cells | yes | | | A1761647 | Homo sapiens clone IMAGE 21721 | 109 | N/A |
| 110 | Resistant cells | | | yes | X80507 | Yes-associated protien 1, 65 kDa | 110 | 306 |
| 111 | Resistant cells | | | | Y16241 | nebulette | 111 | 307 |
| 112 | Resistant cells | | | | D67031 | adducin 3 (gamma) | 112 | 308 |
| 113 | Resistant cells | | | | J05581 | mucin 1, transmembrane | 113 | 309 |
| 114 | Resistant cells | yes | | | U19718 | microfibrillar-associated protein 2 | 114 | 310 |
| 115 | Resistant cells | yes | | | U52840 | sema domain, seven | 115 | 311 |
| | | | | | | thrombospondin repeats (type 1 and | | |
| | | | | | | type 1-like), transmembrane domain | | |
| | | | | | | (TM) and short cytoplasmic | | |
| | | | | | | domain, (semaphorin) 5A | | |
| 116 | Resistant cells | yes | | | AB014557 | KIAA0657 protein | 116 | 312 |
| 117 | Resistant cells | yes | | | AF038172 | hypothetical protein FLJ11149 | 117 | N/A |
| 118 | Resistant cells | | | | AB014529 | A kinase (PRKA) anchor protein 11 | 118 | 313 |
| 119 | Resistant cells | yes | | | U65676 | Hermansky-Pudlak syndrome | 119 | 314 |
| 120 | Resistant cells | yes | | | U14971 | ribosomal protein S9 | 120 | 315 |
| 121 | Resistant cells | yes | | | X74331 | primase, polypeptide 2A (58 kD) | 121 | 316 |
| 122 | Resistant cells | yes | | | D16815 | nuclear receptor subfamily 1, group | 122 | 317 |
| | | | | | | D, member 2 | | |
| 123 | Resistant cells | yes | | | M14333 | Homo sapiens cDNA FLJ32137 fis, | 123 | 318 |
| | | | | | | clone PEBLM2000479, highly | | |
| | | | | | | similar to PROTO-ONCOGENE | | |
| | | | | | | TYROSINE-PROTEIN KINASE | | |
| | | | | | | FYN (EC 2.7.1.112) | | |

TABLE 4

| | | Common to | | | | | | |
|---|---|---|---|---|---|---|---|---|
| | | all 4 BMS | | | | | | |
| | | compounds | | | | | | |
| | Highly | (BMS-A, | Common | Common | | | | SEQ ID |
| | Expressed | BMS- | to | to | | | SEQ ID | NO: of |
| Gene | Cells (Sensitive | B, BMS-C, | BMS-B | BMS-C | Genbank | | NO: of | Amino |
| # | or Resistent) | BMS-D) | Compound | Compound | Accession # | UniGene Title | DNA | Acid |
| 1 | Sensitive cells | yes | | | M22489 | bone morphogenetic protein 2 | 3 | 204 |
| 2 | Sensitive cells | yes | | | AB023194 | KIAA0977 protein | 4 | 205 |
| 3 | Sensitive cells | yes | | | AF009674 | axin | 14 | 215 |
| 4 | Sensitive cells | yes | | | NM_006979 | HLA class II region expressed gene | 2 | 203 |
| | | | | | | KE4 | | |
| 5 | Sensitive cells | | | yes | M28668 | cystic fibrosis transmembrane | 124 | 319 |
| | | | | | | conductance regulator, ATP- | | |
| | | | | | | binding cassette (sub-family C, | | |
| | | | | | | member 7) | | |
| 6 | Sensitive cells | yes | | | AF000560 | Homo sapiens TTF-I interacting | 9 | 210 |
| | | | | | | peptide 20 mRNA, partial cds | | |
| 7 | Sensitive cells | yes | | | M88458 | KDEL (Lys-Asp-Glu-Leu) | 6 | 207 |
| | | | | | | endoplasmic reticulum protein | | |
| | | | | | | retention receptor 2 | | |
| 8 | Sensitive cells | yes | | | AB006622 | KIAA0284 protein | 26 | 226 |
| 9 | Sensitive cells | yes | | | AB014558 | cryptochrome 2 (photolyase-like) | 1 | 202 |
| 10 | Sensitive cells | yes | | | M58603 | nuclear factor of kappa light | 18 | 219 |
| | | | | | | polypeptide gene enhancer in B- | | |
| | | | | | | cells 1 (p105) | | |
| 11 | Sensitive cells | | | yes | W29065 | ESTs, Weakly similar to A28996 | 125 | N/A |
| | | | | | | proline-rich protein M14 precursor - | | |
| | | | | | | mouse [M. musculus] | | |
| 12 | Sensitive cells | yes | | | U03688 | cytochrome P450, subfamily I | 5 | 206 |
| | | | | | | (dioxin-inducible), polypeptide I | | |
| | | | | | | (glaucoma 3, primary infantile) | | |
| 13 | Sensitive cells | yes | | | K03498 | Homo sapiens endogenous | 41 | 241 |
| | | | | | | retrovirus HERV-K104 long | | |
| | | | | | | terminal repeat, complete sequence; | | |
| | | | | | | and Gag protein (gag) and envelope | | |
| | | | | | | protein (env) polynucleotides and | | |
| | | | | | | polypeptides, complete cds | | |
| 14 | Sensitive cells | yes | | | M73077 | glucocorticoid receptor DNA | 15 | 216 |
| | | | | | | binding factor 1 | | |
| 15 | Sensitive cells | yes | | | L40802 | hydroxysteroid (17-beta) | 12 | 213 |
| | | | | | | dehydrogenase 2 | | |
| 16 | Sensitive cells | | | yes | AL050025 | ESTs | 126 | 320 |
| 17 | Sensitive cells | yes | | | AB026891 | solute carrier family 7, (cationic | 30 | 230 |
| | | | | | | amino acid transporter, y+ system) | | |
| | | | | | | member 11 | | |
| 18 | Sensitive cells | | | yes | AF00561 | HIV-1 inducer of short transcripts | 127 | 321 |
| | | | | | | binding protein; lymphoma related | | |
| | | | | | | factor | | |
| 19 | Sensitive cells | yes | | | U15655 | Ets2 repressor factor | 16 | 217 |
| 20 | Sensitive cells | | yes | | U41344 | proline arginine-rich end leucine- | 46 | 246 |
| | | | | | | rich repeat protein | | |
| 21 | Sensitive cells | yes | | | M69023 | tetraspan 3 | 47 | 247 |
| 22 | Sensitive cells | | | yes | AB008515 | retinoic acid repressible protein | 48 | 248 |
| 23 | Sensitive cells | | | | AJ011736 | GRB2-related adaptor protein 2 | 129 | 323 |
| 24 | Sensitive cells | yes | | | U72649 | BTG family, member 2 | 21 | 221 |
| 25 | Sensitive cells | | yes | | U21551 | branched chain aminotransferase 1, | 8 | 209 |
| | | | | | | cytosolic | | |
| 26 | Sensitive cells | yes | | | D13413 | heterogeneous nuclear | 47 | 247 |
| | | | | | | ribonucleoprotein U (scaffold | | |
| | | | | | | attachment factor A) | | |
| 27 | Sensitive cells | yes | | | U19775 | mitogen-activated protein kinase 14 | 59 | 259 |
| 28 | Sensitive cells | | | | J00277 | Human (genomic clones lambda- | 130 | 324 |
| | | | | | | [SK2-T2, HS578T]; cDNA clones | | |
| | | | | | | RS-[3,4, | | |
| 29 | Sensitive cells | yes | | | X77909 | c-Ha-ras 1 proto-oncogene | 23 | 223 |
| 30 | Sensitive cells | | | yes | Y10055 | phosphoinositide-3-kinase, | 131 | 325 |
| | | | | | | catalytic, delta polypeptide | | |
| 31 | Sensitive cells | yes | | | AB029027 | KIAA1104 protein | 27 | 227 |
| 32 | Sensitive cells | yes | | | X60708 | dipeptidylpeptidase IV (CD26, | 34 | 234 |
| | | | | | | adenosine deaminase complexing | | |
| | | | | | | protein 2) | | |
| 33 | Sensitive cells | yes | | | X13916 | low density lipoprotein-related | 13 | 214 |
| | | | | | | protein 1 (alpha-2-macroglobulin | | |
| | | | | | | receptor) | | |
| 34 | Sensitive cells | yes | | | L37033 | FK506-binding protein 8 (38 kD) | 56 | 256 |
| 35 | Sensitive cells | yes | | | AF155654 | Human putative ribosomal protein | 51 | 251 |
| | | | | | | S1 mRNA | | |
| 36 | Sensitive cells | yes | | | L13463 | regulator of G-protein signalling 2, | 7 | 208 |
| | | | | | | 24 kD | | |
| 37 | Sensitive cells | yes | | | AI659108 | Homo sapiens, clone | 20 | N/A |
| | | | | | | IMAGE: 3908182, mRNA, partial | | |
| | | | | | | cds | | |
| 38 | Sensitive cells | | | | W26652 | PTEN induced putative kinase 1 | 132 | N/A |
| 39 | Sensitive cells | yes | | | U51903 | IQ motif containing GTPase | 28 | 228 |
| | | | | | | activating protein 2 | | |
| 40 | Sensitive cells | | | yes | M11717 | heat shock 70 kD protein a 1A | 133 | 326 |
| 41 | Sensitive cells | | | yes | L32976 | mitogen-activated protein kinase | 134 | 327 |
| | | | | | | kinase kinase 11 | | |
| 42 | Sensitive cells | yes | | | L07261 | adducin 1 (alpha) | 54 | 254 |
| 43 | Sensitive cells | | | | M29893 | v-ral simian leukernia viral | 135 | 328 |
| | | | | | | oncogene homolog A (ras related) | | |
| 44 | Sensitive cells | | | | S70154 | acetyl-Coenzyme A | 136 | 329 |
| | | | | | | acetyltranferase 2 (acetoacetyl | | |
| | | | | | | Coenzyme A thiolase) | | |
| 45 | Sensitive cells | | | yes | D83542 | cadherin 15, M-cadherin | 137 | 330 |
| | | | | | | (myotubule) | | |
| 46 | Sensitive cells | | | | Z74615 | collagen, type 1, alpha 1 | 138 | 331 |
| 47 | Sensitive cells | | | yes | M96684 | purine-rich element binding protein A | 139 | 332 |
| 48 | Sensitive cells | yes | | | AF093420 | hsp70-interacting protein | 58 | 258 |
| 49 | Sensitive cells | | | yes | D12763 | interleukin 1 receptor-like 1 | 140 | 333 |
| 50 | Sensitive cells | | | | S67070 | heat shock 27 kD protein 2 | 141 | 334 |
| 51 | Sensitive cells | | yes | | M64571 | microtubule-associated protein 4 | 22 | 222 |
| 52 | Sensitive cells | yes | | | Y09846 | SHC (Src homology 2 domain- | 57 | 257 |
| | | | | | | containing) tranforming protein 1 | | |
| 53 | Sensitive cells | | | | X66435 | 3-hydroxy-3-methylglutaryl- | 142 | 335 |
| | | | | | | Coenzyme A synthase 1 (soluble) | | |
| 54 | Sensitive cells | | | | U25138 | potassium large conductance | 143 | 336 |
| | | | | | | calcium-activated channel, | | |
| | | | | | | subfamily M, beta member 1 | | |
| 55 | Sensitive cells | | | yes | D85131 | MYC-associated zinc finger protein | 144 | 337 |
| | | | | | | (purine-binding transcription factor) | | |
| 56 | Resistant cells | yes | | | AC005329 | NADH dehydrogenase (ubiquinone) | 71 | 270 |
| | | | | | | Fe—S protein 7 (20 kD) (NADH- | | |
| | | | | | | coenzyme Q reductase) | | |
| 57 | Resistant | | yes | | AJ001685 | killer cell lectin-like receptor | 68 | 267 |
| | | | | | | subfamily C, member 3 | | |
| 58 | Resistant cells | yes | | | U90902 | Human clone 23612 mRNA | 66 | N/A |
| | | | | | | sequence | | |
| 59 | Resistant cells | | yes | | X70340 | transforming growth factor, alpha | 73 | 272 |
| 60 | Resistant cells | yes | | | X79067 | zinc finger protein 36, C3H type- | 64 | 264 |
| | | | | | | like 1 | | |
| 61 | Resistant cells | yes | | | Y18483 | solute carrier family 7 (cationic | 61 | 261 |
| | | | | | | amino acid transporter, y+ system), | | |
| | | | | | | member 8 | | |
| 62 | Resistant cells | yes | | | U57352 | amiloride-sensitive cation channel | 62 | 262 |
| | | | | | | 1, neuronal (degenerin) | | |
| 63 | Resistant cells | yes | | | S37730 | insulin-like growth factor binding | 69 | 268 |
| | | | | | | protein 2 (36 kD) | | |
| 64 | Resistant cells | yes | | | D87119 | GS3955 protein | 77 | 276 |
| 65 | Resistant cells | | | yes | M36089 | X-rays repair complementing | 145 | 338 |
| | | | | | | defective repair in Chinese hamster | | |
| | | | | | | cells 1 | | |
| 66 | Resistant cells | yes | | | U34994 | protein kinase, DNA-activated, 63 | 263 | |
| | | | | | | catalytic polypeptide | | |
| 67 | Resistant cells | | | yes | AC004472 | Homo sapiens chromosome 9, P1 | 146 | 339 |
| | | | | | | clone 11659 | | |
| 68 | Resistant cells | | | yes | AB009010 | ubiquitin C | 147 | 340 |
| 69 | Resistant cells | | | | D26158 | ELAV (embryonic lethal, abnormal | 148 | 341 |
| | | | | | | vision, Drosophila)-like 3 (Hu | | |
| | | | | | | antigen C) | | |
| 70 | Resistant cells | yes | | | AF007156 | KIAA0751 gene product | 82 | 281 |
| 71 | Resistant cells | yes | | | AF031824 | cystatin F (leukocystatin) | 88 | 287 |
| 72 | Resistant cells | yes | | | AA595596 | ADP-ribosyltransferase (NAD+; | 104 | N/A |
| | | | | | | poly(ADP-ribose) polymerase)-like 2 | | |
| 73 | Resistant cells | | | yes | AL035307 | H. sapiens gene from PAC 42616 | 149 | 342 |
| 74 | Resistant cells | yes | | | AB000449 | vaccinia related kinase 1 | 76 | 275 |
| 75 | Resistant cells | yes | | | AF070530 | hypothetical protein, clone 24751 | 80 | 279 |
| 76 | Resistant cells | yes | | | Ab011535 | FAT tumor suppressor (Drosophila) | 65 | 265 |
| | | | | | | homolog 2 | | |
| 77 | Resistant cells | yes | | | AB014566 | KIAA0666 protein | 83 | 282 |
| 78 | Resistant cells | yes | | | U81561 | protein tyrosine phosphatase, | 74 | 273 |
| | | | | | | receptor type, N polypeptide 2 | | |
| 79 | Resistant cells | yes | | | M31682 | inhibin, beta B (activin AB beta | 87 | 286 |
| | | | | | | polypeptide) | | |
| 80 | Resistant cells | yes | | | X90976 | runt-related transcription factor 1 | 93 | 292 |
| | | | | | | (acute myeloid leukemia 1; aml1 | | |
| | | | | | | oncogene) | | |
| 81 | Resistant cells | yes | | | X06745 | polymerase (DNA directed), alpha | 78 | 277 |
| 82 | Resistant cells | | | yes | AL043470 | hypothetical protein FLJ10335 | 150 | N/A |
| 83 | Resistant cells | yes | | | U84570 | chromosome 21 open reading frame 2 | 91 | 290 |
| 84 | Resistant cells | yes | | | L34059 | cadherin 4, type 1, R-cadherin | 100 | 299 |
| | | | | | | (retinal) | | |
| 85 | Resistant cells | yes | | | U19718 | microfibrillar-associated protein 2 | 114 | 310 |
| 86 | Resistant cells | yes | | | U52840 | sema domain, seven | 115 | 311 |
| | | | | | | thrombospondin repeats (type 1 and | | |
| | | | | | | type 1-like), transmembrane domain | | |
| | | | | | | (TM) and short cytoplasmic | | |
| | | | | | | domain, (semaphorin) 5A | | |
| 87 | Resistant cells | yes | | | L07540 | replication factor C (activator 1) 5 | 97 | 296 |
| | | | | | | (36.5 kD) | | |
| 88 | Resistant cells | | | yes | U67733 | phosphodiesterase 2A, cGMP- | 151 | 343 |
| | | | | | | stimulated | | |
| 89 | Resistant cells | | | | D28118 | zinc finger protein 161 | 152 344 | |
| 90 | Resistant cells | | | yes | AB020661 | KIAA0854 protein | 153 | 345 |
| 91 | Resistant cells | yes | | | U68111 | protein phosphatase 1, regulatory | 96 | 295 |
| | | | | | | (inhibitor) subunit 2 | | |
| 92 | Resistant cells | | | | W72186 | S100 calcium-binding protein A4 | 154 | 346 |
| | | | | | | (calcium protein, calvasculin, | | |
| | | | | | | metastasin, murine placental | | |
| | | | | | | homolog) | | |
| 93 | Resistant cells | yes | | | L43821 | enhancer of filamentation 1 (cas- | 81 | 280 |
| | | | | | | like docking; Crk-associated | | |
| | | | | | | substrate related) | | |
| 94 | Resistant cells | | | | U97067 | catenin (cadherin-associated | 155 | 347 |
| | | | | | | protein), alpha-like 1 | | |
| 95 | Resistant cells | yes | | | AF003837 | jagged 1 (Alagille syndrome) | 105 | 303 |
| 96 | Resistant cells | yes | | | AF038172 | hypothetical protein FLJ11149 | 117 | N/A |
| 97 | Resistant cells | | | | AF061261 | hypothetical protein PRO2032 | 156 | 348 |
| 98 | Resistant cells | yes | | | AB018306 | KIAA0763 gene product | 108 | 305 |
| 99 | Resistant cells | yes | | | M14333 | Homo sapiens cDNA FLJ32137 fis, | 123 | 318 |
| | | | | | | clone PEBLM2000479, highly | | |
| | | | | | | similar to PROTO-ONCOGENE | | |
| | | | | | | TYROSINE-PROTEIN KINASE | | |
| | | | | | | FYN (EC 2.71.1.112) | | |
| 100 | Resistant cells | | | yes | AB007870 | KIAA0410 gene product | 157 | 349 |
| 101 | Resistant cells | yes | | | AB014557 | KIAA0657 protein | 116 | 312 |
| 102 | Resistant cells | | yes | | AB009426 | apolipoprotein B mRNA editing | 72 | 271 |
| | | | | | | enzyme, catalytic polypetide 1 | | |
| 103 | Resistant cells | | yes | | U37518 | tumor necrosis factor (ligand) | 70 | 269 |
| | | | | | | superfamily, member 10 | | |
| 104 | Resistant cells | yes | | | X78817 | Rho GTPase activating protein 4 | 79 | 278 |
| 105 | Resistant cells | yes | | | AB006626 | histone deacetylase 4 | 86 | 285 |
| 106 | Resistant cells | | | | AB014519 | Rho-associated, coiled-coil | 158 | 350 |
| | | | | | | containing protein kinase 2 | | |
| 107 | Resistant cells | yes | | | AL050290 | spermidine/spermine N1- | 102 | 301 |
| | | | | | | acetyltransferase | | |
| 108 | Resistant cells | yes | | | D89377 | msh (Drosophila) homeo box | 94 | 293 |
| | | | | | | homolog 2 | | |
| 109 | Resistant cells | yes | | | X74331 | primase, polypeptide 2A (58 kD) | 121 | 316 |
| 110 | Resistant cells | | yes | | AF035299 | docking protein 1, 62 kD | 89 | 288 |
| | | | | | | (downstream of tyrosine kinase 1) | | |
| 111 | Resistant cells | | yes | | X70040 | macrophage stimulating 1 receptor | 75 | 274 |
| | | | | | | (c-met-related tyrosine kinase) | | |
| 112 | Resistant cells | yes | | | U14971 | ribosomal protein S9 | 120 | 315 |
| 113 | Resistant cells | yes | | | U65676 | Hermansky-Pudlak syndrome | 119 | 314 |
| 114 | Resistant cells | | | | AB011123 | KIAA0551 protein | 159 | 351 |
| 115 | Resistant cells | | | | M34182 | protein kinase, cAMP-dependent, | 99 | 298 |
| | | | | | | catalytic, gamma | | |
| 116 | Resistant cells | yes | | | D16815 | nuclear receptor subfamily 1, group | 122 | 317 |
| | | | | | | D, member 2 | | |
| 117 | Resistant cells | yes | | | AI761647 | Homo sapiens clone IMAGE 21721 | 109 | N/A |
| 118 | Resistant cells | | | | U09578 | mitogen-activated protein kinase- | 160 | 352 |
| | | | | | | activated protein kinase 3 | | |
| 119 | Resistant cells | yes | | | M87339 | replication factor C (activator 1) 4 | 106 | 304 |
| | | | | | | (37 kD) | | |

TABLE 5

| | | Common to all | | | | | | |
|---|---|---|---|---|---|---|---|---|
| | | 4 BMS | | | | | | |
| | Highly | compounds | Common to | | | | | SEQ ID |
| | Expressed | (BMS-A, BMS- | BMS-A/ | Common to | | | SEQ ID | NO: of |
| | Cells (Sensitive | B, BMS-C, | BMS-D | BMS-B | Genbank | | NO: of | Amino |
| Gene # | or Resistent) | BMS-D) | Compound | Compound | Accession # | UniGene Title | DNA | Acid |
| 1 | Sensitive cells | yes | | | M22489 | bone morphogenetic protein 2 | 3 | 204 |
| 2 | Sensitive cells | yes | | | AF009674 | axin | 14 | 215 |
| 3 | Sensitive cells | | | yes | D12763 | interleukin 1 receptor-like 1 | 140 | 333 |
| 4 | Sensitive cells | yes | | | AF000560 | Homo sapiens TTF-I interacting | 9 | 210 |
| | | | | | | peptide 20 mRNA, partial cds | | |
| 5 | Sensitive cells | yes | | | AB014558 | cryptochrome 2 (photolyase-like) | 1 | 202 |
| 6 | Sensitive cells | | | yes | M28668 | cystic fibrosis transmembrane | 124 | 319 |
| | | | | | | conductance regulator, ATP- | | |
| | | | | | | binding cassette (sub-family C, | | |
| | | | | | | member 7) | | |
| 7 | Sensitive cells | yes | | | M88458 | kDEL (Lys-Asp-Glu-Leu) | 6 | 207 |
| | | | | | | endoplasmic reticulum protein | | |
| | | | | | | retention receptor 2 | | |
| 8 | Sensitive cells | | | | Y17711 | calcium binding atopy-related | 161 | 353 |
| | | | | | | autoantigen 1 | | |
| 9 | Sensitve cells | yes | | | M69023 | tetraspan 3 | 48 | 248 |
| 10 | Sensitive cells | | | | LI3972 | sialytransferase 4A (beta- | 162 | 354 |
| | | | | | | galactosidase alpha-2,3- | | |
| | | | | | | sialytransferase) | | |
| 11 | Sensitive cells | yes | | | AB006622 | KIAA0284 protein | 26 | 226 |
| 12 | Sensitive cells | | | | AF055009 | old astrocyte specifically induced | 163 | 355 |
| | | | | | | substance | | |
| 13 | Sensitive cells | | yes | | X06272 | signal recognition particle receptor | 11 | 212 |
| | | | | | | (‘docking protein’) | | |
| 14 | Sensitive cells | | | yes | W29065 | ESTs, Weakly similar to A28996 | 125 | N/A |
| | | | | | | proline-rich protein M14 precursor - | | |
| | | | | | | mouse [M. musculus] | | |
| 15 | Sensitive cells | yes | | | AB023194 | KIAA0977 protein | 4 | 205 |
| 16 | Sensitive cells | | | | AF007155 | Homo sapiens clone 23763 | 164 | 356 |
| | | | | | | unknown mRNA, partial cds | | |
| 17 | Sensitive cells | yes | | | U19775 | mitogen-activated protein kinase 14 | 59 | 259 |
| 18 | Sensitive cells | yes | | | U03688 | cytochrome P450, subfamily I | 5 | 206 |
| | | | | | | (dioxin-inducible), polypeptide 1 | | |
| | | | | | | (glaucoma 3, primary infantile) | | |
| 19 | Sensitive cells | | | | AB018324 | KIAA0781 protein | 165 | 357 |
| 20 | Sensitive cells | | | yes | AF00561 | HIV-1 inducer of short transcripts | 127 | 321 |
| | | | | | | binding protein; lymphoma related | | |
| | | | | | | factor | | |
| 21 | Sensitive cells | | | | AB023154 | KIAA0937 protein | 166 | 358 |
| 22 | Sensitive cells | | | | X78992 | zinc finger protein 36, C3H type- | 167 | 359 |
| | | | | | | like 2 | | |
| 23 | Sensitive cells | yes | | | L40802 | hydroxysteriod (17-beta) | 12 | 213 |
| | | | | | | dehydrogenase 2 | | |
| 24 | Sensitive cells | yes | | | M73077 | glucocorticoid receptor DNA | 15 | 216 |
| | | | | | | binding factor 1 | | |
| 25 | Sensitive cells | yes | | | AF155654 | Human putative ribosomal protein | 51 | 251 |
| | | | | | | S1 mRNA | | |
| 26 | Sensitive cells | yes | | | U15655 | Ets2 repressor factor | 16 | 217 |
| 27 | Sensitive cells | | | | M60299 | collagen, type II, alpha 1 (primary | 168 | 360 |
| | | | | | | osteoarthritis, spondyleopiphyseal | | |
| | | | | | | dysplasia, congenital) | | |
| 28 | Sensitive cells | | | yes | AL050025 | ESTs | 126 | 320 |
| 29 | Sensitive cells | | | | M80482 | paired basic amino acid cleaving | 169 | 361 |
| | | | | | | system 4 | | |
| 30 | Sensitive cells | yes | | | X60708 | dipeptidylpeptidase IV (CD26, | 34 | 234 |
| | | | | | | adenosine deaminase complexing | | |
| | | | | | | protein 2) | | |
| 31 | Sensitive cells | | | | U47025 | ESTs, Moderately similar to | 170 | 362 |
| | | | | | | 1701409A glycogen phosphorylase | | |
| | | | | | | [H. Sapiens] | | |
| 32 | Sensitive cells | | | yes | Y10055 | phosphoinositide-3-kinase, | 131 | 325 |
| | | | | | | catalytic, delta polypeptide | | |
| 33 | Sensitive cells | yes | | | L13463 | regulator of G-protein signalling 2, 24 kD | 7 | 208 |
| 34 | Sensitive cells | | | | Y10032 | serum/glucocorticoid regulated | 171 | 363 |
| | | | | | | kinase | | |
| 35 | Sensitive cells | yes | | | X77909 | nuclear factor of kappa light | 23 | 223 |
| | | | | | | polypeptide gene enhancer in B- | | |
| | | | | | | cells inhibitor-like 1 | | |
| 36 | Sensitive cells | yes | | | U51903 | IQ motif containing GTPase | 28 | 228 |
| | | | | | | activating protein 2 | | |
| 37 | Sensitive cells | | | | U57057 | coronin, actin-binding protein, 2A | 172 | 364 |
| 38 | Sensitive cells | yes | | | K03498 | Homo sapiens endogenous | 41 | 241 |
| | | | | | | retrovirus HER V-K104 long | | |
| | | | | | | terminal repeat, complete sequence; | | |
| | | | | | | and Gag protein (gag) and envelope | | |
| | | | | | | protein (env) polynucleotides and | | |
| | | | | | | polypetides | | |
| 39 | Sensitive cells | | | | X90392 | deoxyribonuclease I-like 1 | 173 | 365 |
| 40 | Sensitive cells | | | yes | L32976 | mitogen-activated protein kinase | 134 | 327 |
| | | | | | | kinase kinase 11 | | |
| 41 | Sensitive cells | | | yes | M96684 | purine-rich element binding protein A | 139 | 332 |
| 42 | Sensitive cells | | | yes | D83542 | cadherin 15, M-cadherin | 137 | 330 |
| | | | | | | (myotubule) | | |
| 43 | Sensitive cells | yes | | | AB026891 | solute carrier family 7, (cationic | 30 | 230 |
| | | | | | | amino acid transporter, y+ system) | | |
| | | | | | | member 11 | | |
| 44 | Sensitive cells | | | | AA418437 | chromosome 1 open reading frame 27 | 174 | N/A |
| 45 | Sensitive cells | | | yes | M11717 | heat shock 70 kD protein 1A | 133 | 326 |
| 46 | Sensitive cells | yes | | | AI659108 | Homo sapiens, clone | 20 | N/A |
| | | | | | | IMAGE: 3908182, mRNA, partial | | |
| | | | | | | cds | | |
| 47 | Sensitive cells | yes | | | L07261 | adducin 1 (alpha) | 54 | 254 |
| 48 | Sensitive cells | yes | | | AF093420 | hsp70-interacting protein | 58 | 258 |
| 49 | Sensitive cells | yes | | | D13413 | heterogeneous nuclear | 47 | 247 |
| | | | | | | ribonucleoprotein U (scaffold | | |
| | | | | | | attachment factor A) | | |
| 50 | Sensitive cells | | | yes | AB008515 | retinoic acid repressible protein | 128 | 322 |
| 51 | Sensitive cells | | | | X84373 | nuclear receptor interacting protein 1 | 175 | 366 |
| 52 | Sensitive cells | | | | XM_212189 | Homo sapiens glutamate receptor, | 176 | 367 |
| | | | | | | ionotropic, N-methyl D-asparate- | | |
| | | | | | | associated protein 1 (glutamate | | |
| | | | | | | binding) (GRINA), mRNA | | |
| 53 | Sensitive cells | yes | | | U72649 | D-asparate-associated protein 1 | 21 | 221 |
| | | | | | | (glutamate binding) (GRINA), | | |
| | | | | | | mRNA | | |
| 54 | Sensitive cells | yes | | | NM_006979 | HLA class II region expressed gene | 2 | 203 |
| | | | | | | KE4 | | |
| 55 | Sensitive cells | yes | | | M58603 | nuclear factor of kappa light | 18 | 219 |
| | | | | | | polypeptide gene enhancer in B- | | |
| | | | | | | cells 1 (p105) | | |
| 56 | Sensitive cells | | | | AF109134 | opioid growth factor receptor | 177 | 368 |
| 57 | Sensitive cells | yes | | | L37033 | FK506-binding protein 8 (38 kD) | 56 | 256 |
| 58 | Sensitive cells | | | | M64788 | RAPI, GTPase activating protein 1 | 178 | 369 |
| 59 | Sensitive cells | | | | U43368 | vascular endothelial growth factor B | 179 | 370 |
| 60 | Sensitive cells | yes | | | AB029027 | KIAA1104 protein | 27 | 227 |
| 61 | Sensitive cells | | | | X13293 | v-myb avain myeloblastosis viral | 180 | 371 |
| | | | | | | oncogene homolog-like 2 | | |
| 62 | Sensitive cells | | | yes | D85131 | MYC-associated zinc finger protein | 144 | 337 |
| | | | | | | (purine-binding transcription factor) | | |
| 63 | Sensitive cells | | | | AB014511 | ATPase, Class II, type 9A | 181 | 372 |
| 64 | Sensitive cells | yes | | | X13916 | low density lipoprotein-related | 13 | 214 |
| | | | | | | protein 1 (alpha-2-macroglobulin | | |
| | | | | | | receptor) | | |
| 65 | Sensitive cells | yes | | | Y09846 | SHC (Src homology 2 domain- | 57 | 257 |
| | | | | | | containing) transforming protein 1 | | |
| 66 | Resistant cells | | | yes | AC004472 | Homo sapiens chromosome 9, P1 | 146 | 339 |
| | | | | | | clone 11659 | | |
| 67 | Resistant cells | yes | | | U90902 | Human clone 23612 mRNA | 66 | N/A |
| | | | | | | sequence | | |
| 68 | Resistant cells | | | | AB014585 | KIAA0685 gene product | 182 | 373 |
| 69 | Resistant cells | yes | | | X06745 | polymerase (DNA directed), alpha | 78 | 277 |
| 70 | Resistant cells | | | | AB011114 | K1AA0542 gene product | 183 | 374 |
| 71 | Resistant cells | | | yes | AL035307 | H. sapiens gene from PAC 42616 | 149 | 342 |
| 72 | Resistant cells | yes | | | S37730 | insulin-like growth factor binding | 69 | 268 |
| | | | | | | protein 2 (36 kD) | | |
| 73 | Resistant cells | | | yes | M36089 | X-ray repair complementing | 145 | 338 |
| | | | | | | defective repair in Chinese hamster | | |
| | | | | | | cells 1 | | |
| 74 | Resistant cells | yes | | | AB000449 | vaccinia related kinase 1 | 76 | 275 |
| 75 | Resistant cells | yes | | | U34994 | protein kinase, DNA-activated, | 63 | 263 |
| | | | | | | catalytic polypeptide | | |
| 76 | Resistant cells | yes | | | AA595596 | ADP-ribosyltransferase (NAD+; | 104 | N/A |
| | | | | | | poly (ADP-ribose) polymerase)-like 2 | | |
| 77 | Resistant cells | | | yes | AB009010 | ubiquitin C | 147 | 340 |
| 78 | Resistant cells | yes | | | Y18483 | solute carrier family 7 (cationic | 61 | 261 |
| | | | | | | amino acid transporter, y+ system), | | |
| | | | | | | member 8 | | |
| 79 | Resistant cells | yes | | | U84570 | chromosome 21 open reading frame 2 | 91 | 290 |
| 80 | Resistant cells | yes | | | AF007156 | KIAA0751 gene product | 82 | 281 |
| 81 | Resistant cells | | | yes | AB007870 | KIAA0410 gene product | 157 | 349 |
| 82 | Resistant cells | | | | U80040 | aconitase 2, mitochondrial | 184 | 375 |
| 83 | Resistant cells | | | | M64174 | Janus kinase 1 (a protein tyrosine | 185 | 376 |
| | | | | | | kinase) | | |
| 84 | Resistant cells | yes | | | AB011535 | FAT tumor suppressor (Drosophila) | 65 | 265 |
| | | | | | | homolog 2 | | |
| 85 | Resistant cells | yes | | | AC005329 | NADH dehydrogenase (ubiquinone) | 71 | 270 |
| | | | | | | Fe—S protein 7 (20 kD) (NADH- | | |
| | | | | | | coenzyme Q reductase) | | |
| 86 | Resistant cells | yes | | | AF038172 | hypothetical protein FLJ11149 | 117 | N/A |
| 87 | Resistant cells | yes | | | U68111 | protein phosphatase 1, regulatory | 96 | 295 |
| | | | | | | (inhibitor) subunit 2 | | |
| 88 | Resistant cells | | yes | | U71364 | serine (or cysteine) proteinase | 84 | 283 |
| | | | | | | inhibitor, clade B (ovalbumin), | | |
| | | | | | | member 9 | | |
| 89 | Resistant cells | yes | | | D89377 | msh (Drosophila) homeo box | 94 | 293 |
| | | | | | | homolog 2 | | |
| 90 | Resistant cells | | | | Y00971 | phosphoribosyl pyrophosphate | 186 | 377 |
| | | | | | | synthetase 2 | | |
| 91 | Resistant cells | | | | AL050065 | DNA DKFZp566M043 (from clone | 187 | N/A |
| | | | | | | DKFZp566M043) | | |
| 92 | Resistant cells | | | | AF029670 | RAD51 (S. cerevisiae) homolog C | 188 | 378 |
| 93 | Resistant cells | yes | | | M31682 | inhibin, beta B (activin AB beta | 87 | 286 |
| | | | | | | polypeptide) | | |
| 94 | Resistant cells | | | | X63629 | cadherin 3, type 1, P-cadherin | 189 | 379 |
| | | | | | | (placental) | | |
| 95 | Resistant cells | | | | AB028957 | KIAA1034 protein | 190 | 380 |
| 96 | Resistant cells | yes | | | D87119 | GS3955 protein | 77 | 276 |
| 97 | Resistant cells | yes | | | M14333 | Homo sapiens cDNA FLJ32137 fis, | 123 | 318 |
| | | | | | | clone PEBLM2000479, highly | | |
| | | | | | | similar to PROTO-ONCOGENE | | |
| | | | | | | TYROSINE-PROTEIN KINASE | | |
| | | | | | | FYN (EC 2.7.1.112) | | |
| 98 | Resistant cells | yes | | | L07540 | replication factor C (activator 1) 5 | 97 | 296 |
| | | | | | | (36.5 kD) | | |
| 99 | Resistant cells | | | | X74837 | mannosidase, alpha, class 1A, | 191 | 381 |
| | | | | | | member 1 | | |
| 100 | Resistant cells | yes | | | X90976 | runt-related transcription factor 1 | 93 | 292 |
| | | | | | | (acute myeloid leukemia 1; aml 1 | | |
| | | | | | | oncogene) | | |
| 101 | Resistant cells | | | | AB018273 | collagen, type 1, alpha 2 | 192 | 382 |
| 102 | Resistant cells | yes | | | AF003837 | jagged 1 (Alagille syndrome) | 105 | 303 |
| 103 | Resistant cells | yes | | | AL050290 | spermidine/spermine N1- | 102 | 301 |
| | | | | | | acetyltransferase | | |
| 104 | Resistant cells | yes | | | AB014566 | KIAA0666 protein | 83 | 282 |
| 105 | Resitant cells | | | | Y15227 | deleted in lymphocytic leukemia, 1 | 193 | 383 |
| 106 | Resistant cells | yes | | | X79067 | zinc finger protein 36, C3H type- | 64 | 264 |
| | | | | | | like 1 | | |
| 107 | Resistant cells | yes | | | AF031824 | cystatin F (leukocystatin) | 88 | 287 |
| 108 | Resistant cells | | | | Y12661 | VGF nerver growth factor inducible | 194 | 384 |
| 109 | Resistant cells | yes | | | X74331 | primase, polypeptide 2A (58 kD) | 121 | 316 |
| 110 | Resistant cells | | | yes | AL043470 | hypothetical protein FLJ10335 | 150 | N/A |
| 111 | Resistant cells | | | yes | U67733 | phosphodiesterase 2A, cGMP- | 151 | 343 |
| | | | | | | stimulated | | |
| 112 | Resistant cells | | | | AL049365 | Homo sapiens mRNA; cDNA | 195 | N/A |
| | | | | | | DKFZp586A0618 (from clone | | |
| | | | | | | DKFZp586A0618) | | |
| 113 | Resistant cells | yes | | | L43821 | enhancer of filamentation 1 (cas- | 81 | 280 |
| | | | | | | like docking; Crk-associated | | |
| | | | | | | substrate related) | | |
| 114 | Resistant cells | | | | Y11395 | LanC (bacterial lantibiotic | 196 | 385 |
| | | | | | | synthetase components C)- like 1 | | |
| 115 | Resistant cells | yes | | | L34059 | cadherin 4, type 1, R-cadherin | 100 | 299 |
| | | | | | | (retinal) | | |
| 116 | Resistant cells | | | | NM_004713 | serologically defined colon cancer | 197 | 386 |
| | | | | | | antigen 1 | | |
| 117 | Resistant cells | yes | | | AB006626 | histone deacetylase 4 | 86 | 285 |
| 118 | Resistant cells | | | | X76029 | neuromedin U | 198 | 387 |
| 119 | Resistant cells | yes | | | U81561 | protein tyrosine phosphatase, | 74 | 273 |
| | | | | | | receptor type, N polypeptide 2 | | |
| 120 | Resistant cells | yes | | | M87339 | replication factor C (activator 1) 4 | 106 | 304 |
| | | | | | | (37 kD) | | |
| 121 | Resistant cells | yes | | | U57352 | amiloride-sensitive cation channel | 62 | 262 |
| | | | | | | 1, neuronal (degenerin) | | |
| 121 | Resistant cells | yes | | | U57352 | amiloride-sensitive cation channel | 62 | 262 |
| | | | | | | 1, neuronal (degenerin) | | |
| 122 | Resistant cells | yes | | | AF070530 | hypothetical protein, clone 24751 | 80 | 279 |
| 123 | Resistant cells | yes | | | U19718 | microfibrillar-associated protein 2 | 114 | 310 |
| 124 | Resistant cells | yes | | | U65676 | Hermansky-Pudlak syndrome | 119 | 314 |
| 125 | Resistant cells | yes | | | U52840 | sema domain, seven | 115 | 311 |
| | | | | | | thrombospondin repeats (type 1 and | | |
| | | | | | | type 1-like), transmembrane domain | | |
| | | | | | | (TM) and short cytoplasmic | | |
| | | | | | | domain, (semaphorin) 5A | | |
| 126 | Resistant cells | | | | AF045583 | tubby like protein 3 | 199 | 388 |
| 127 | Resistant cells | | | | X85545 | protein kinase, X-linked | 200 | 389 |
| 128 | Resistant cells | yes | | | AB014557 | KIAA0657 protein | 116 | 312 |
| 129 | Resistant cells | yes | | | M34182 | protein kinase, cAMP-dependent, | 99 | 298 |
| | | | | | | catalytic, gamma | | |
| 130 | Resistant cells | | | | X12534 | RAP2A, member of RAS oncogene | 201 | 390 |
| | | | | | | family | | |
| 131 | Resistant cells | | | yes | AB020661 | KIAA0854 protein | 153 | 345 |
| 132 | Resistant cells | yes | | | A1761647 | Homo sapiens clone IMAGE 21721 | 109 | N/A |
| 133 | Resistant cells | | yes | | X80507 | Yes-associated protein 1, 65 kDa | 110 | 306 |
| 134 | Resistant cells | yes | | | U14971 | ribosomal protein S9 | 120 | 315 |
| 135 | Resistant cells | yes | | | X78817 | Rho GTPase activating protein 4 | 79 | 278 |
| 136 | Resistant cells | yes | | | D16815 | nuclear receptor subfamily 1, group | 122 | 317 |
| | | | | | | D, member 2 | | |
| 137 | Resistant cells | yes | | | AB018306 | KIAA0763 gene product | 108 | 305 |

TABLE 6

| | Highly | | | | SEQ ID |
|---|---|---|---|---|---|
| | Expressed Cells | | | SEQ ID | NO: of |
| | (Sensitive or | Genbank | | NO: of | Amino |
| Gene # | Resistent | Accession # | UniGene Title | DNA | Acid |
| 1 | Sensitive cells | AB014558 | cryptochrome 2 (photolyase- | 1 | 202 |
| | | | like) | | |
| 2 | Sensitive cells | NM_006979 | HLA class II region expressed | 2 | 203 |
| | | | gene KE4 | | |
| 3 | Sensitive cells | M22489 | bone morphogenetic protein 2 | 3 | 204 |
| 4 | Sensitive cells | AB023194 | KIAA0977 protein | 4 | 205 |
| 5 | Sensitive cells | U03688 | cytochrome P450, subfamily I | 5 | 206 |
| | | | (dioxin-inducible), polypeptide | | |
| | | | 1 (glaucoma 3, primary | | |
| | | | infantile) | | |
| 6 | Sensitive cells | M88458 | KDEL (Lys-Asp-Glu-Leu) | 6 | 207 |
| | | | endoplasmic reticulum protein | | |
| | | | retention receptor 2 | | |
| 7 | Sensitive cells | L13463 | regulator of G-protein signalling | 7 | 208 |
| | | | 2, 24 kD | | |
| 8 | Sensitive cells | AF000560 | Homo sapiens TTF-I interacting | 9 | 210 |
| | | | peptide 20 mRNA, partial cds | | |
| 9 | Sensitive cells | L40802 | hydroxysteroid (17-beta) | 12 | 213 |
| | | | dehydrogenase 2 | | |
| 10 | Sensitive cells | X13916 | low density lipoprotein-related | 13 | 214 |
| | | | protein 1 (alpha-2- | | |
| | | | macroglobulin receptor) | | |
| 11 | Sensitive cells | AF009674 | axin | 14 | 215 |
| 12 | Sensitive cells | M73077 | glucocorticoid receptor DNA | 15 | 216 |
| | | | binding factor 1 | | |
| 13 | Sensitive cells | U15655 | Ets2 repressor factor | 16 | 217 |
| 14 | Sensitive cells | M58603 | nuclear factor of kappa light | 18 | 219 |
| | | | polypeptide gene enhancer in B- | | |
| | | | cells 1 (p105) | | |
| 15 | Sensitive cells | AI659108 | Homo sapiens, clone | 20 | N/A |
| | | | IMAGE:3908182, mRNA, | | |
| | | | partial cds | | |
| 16 | Sensitive cells | U72649 | BTG family, member 2 | 21 | 221 |
| 17 | Sensitive cells | X77909 | nuclear factor of kappa light | 23 | 223 |
| | | | polypeptide gene enhancer in B- | | |
| | | | cells inhibitor-like 1 | | |
| 18 | Sensitive cells | AB006622 | KIAA0284 protein | 26 | 226 |
| 19 | Sensitive cells | AB029027 | KIAA1104 protein | 27 | 227 |
| 20 | Sensitive cells | U51903 | IQ motif containing GTPase | 28 | 228 |
| | | | activating protein 2 | | |
| 21 | Sensitive cells | AB026891 | solute carrier family 7, (cationic | 30 | 230 |
| | | | amino acid transporter, y+ | | |
| | | | system) member 11 | | |
| 22 | Sensitive cells | X60708 | dipeptidylpeptidase IV (CD26, | 34 | 234 |
| | | | adenosine deaminase | | |
| | | | complexing protein 2) | | |
| 23 | Sensitive cells | K03498 | Homo sapiens endogenous | 41 | 241 |
| | | | retrovirus HERV-K104 long | | |
| | | | terminal repeat, complete | | |
| | | | sequence; and Gag protein (gag) | | |
| | | | and envelope protein (env) | | |
| | | | polynucleotides and | | |
| | | | polypeptides, complete cds | | |
| 24 | Sensitive cells | D13413 | heterogeneous nuclear | 47 | 247 |
| | | | ribonucleoprotein U (scaffold | | |
| | | | attachment factor A) | | |
| 25 | Sensitive cells | M69023 | tetraspan 3 | 48 | 248 |
| 26 | Sensitive cells | AF155654 | Human putative ribosomal | 51 | 251 |
| | | | protein S1 mRNA | | |
| 27 | Sensitive cells | L07261 | adducin 1 (alpha) | 54 | 254 |
| 28 | Sensitive cells | L37033 | FK506-binding protein 8 (38 kD) | 56 | 256 |
| 29 | Sensitive cells | Y09846 | SHC (Src homology 2 domain- | 57 | 257 |
| | | | containing) transforming protein 1 | | |
| 30 | Sensitive cells | AF093420 | hsp70-interacting protein | 58 | 258 |
| 31 | Sensitive cells | U19775 | mitogen-activated protein kinase 14 | 59 | 259 |
| 32 | Resistant cells | Y18483 | solute carrier family 7 (cationic | 61 | 261 |
| | | | amino acid transporter, y+ | | |
| | | | system), member 8 | | |
| 33 | Resistant cells | U57352 | amiloride-sensitive cation | 62 | 262 |
| | | | channel 1, neuronal (degenerin) | | |
| 34 | Resistant cells | U34994 | protein kinase, DNA-activated, | 63 | 263 |
| | | | catalytic polypeptide | | |
| 35 | Resistant cells | X79067 | zinc finger protein 36, C3H | 64 | 264 |
| | | | type-like 1 | | |
| 36 | Resistant cells | AB011535 | FAT tumor suppressor | 65 | 265 |
| | | | (Drosophila) homolog 2 | | |
| 37 | Resistant cells | U90902 | Human clone 23612 mRNA | 66 | N/A |
| | | | sequence | | |
| 38 | Resistant cells | S37730 | insulin-like growth factor | 69 | 268 |
| | | | binding protein 2 (36 kD) | | |
| 39 | Resistant cells | AC005329 | NADH dehydrogenase | 71 | 270 |
| | | | (ubiquinone) Fe—S protein 7 | | |
| | | | (20 kD) (NADH-coenzyme Q | | |
| | | | reductase) | | |
| 40 | Resistant cells | U81561 | protein tyrosine phosphatase, | 74 | 273 |
| | | | receptor type, N polypeptide 2 | | |
| 41 | Resistant cells | AB000449 | vaccinia related kinase 1 | 76 | 275 |
| 42 | Resistant cells | D87119 | GS3955 protein | 77 | 276 |
| 43 | Resistant cells | X06745 | polymerase (DNA directed), | 78 | 277 |
| | | | alpha | | |
| 44 | Resistant cells | X78817 | Rho GTPase activating protein 4 | 79 | 278 |
| 45 | Resistant cells | AF070530 | hypothetical protein, clone | 80 | 279 |
| | | | 24751 | | |
| 46 | Resistant cells | L43821 | enhancer of filamentation 1 (case- | 81 | 580 |
| | | | like docking; Crk-associated | | |
| | | | substrate related) | | |
| 47 | Resistant cells | AF007156 | KIAA0751 gene product | 82 | 281 |
| 48 | Resistant cells | AB014566 | KIAA0666 protein | 83 | 282 |
| 49 | Resistant cells | AB006626 | histone deacetylase 4 | 86 | 285 |
| 50 | Resistant cells | M31682 | inhibin, beta B (activin AB beta | 87 | 286 |
| | | | polypeptide) | | |
| 51 | Resistant cells | AF031824 | cystatin F (leukocystatin) | 88 | 287 |
| 52 | Resistant cells | U84570 | chromosome 21 open reading | 91 | 290 |
| | | | frame 2 | | |
| 53 | Resistant cells | X90976 | runt-related transcription factor | 93 | 292 |
| | | | 1 (acute myeloid leukemia 1; | | |
| | | | am11 oncogene) | | |
| 54 | Resistant cells | D89377 | msh (Drosophila) homeo box | 94 | 293 |
| | | | homolog 2 | | |
| 55 | Resistant cells | L07540 | replication factor C (activator 1) | 97 | 296 |
| | | | 5 (36.5 kD) | | |
| 56 | Resistant cells | M34182 | protein kinase, cAMP- | 99 | 298 |
| | | | dependent, catalytic, gamma | | |
| 57 | Resistant cells | L34059 | cadherin 4, type 1, R-cadherin | 100 | 299 |
| | | | (retinal) | | |
| 58 | Resistant cells | AL050290 | spermidine/spermine N1- | 102 | 301 |
| | | | acetyltransferase | | |
| 59 | Resistant cells | AA595596 | ADP-ribosyltransferase (NAD+; | 104 | N/A |
| | | | poly(ADP-ribose) polymerase)- | | |
| | | | like 2 | | |
| 60 | Resistant cells | AF003837 | jagged 1 (Alagille syndrome) | 105 | 303 |
| 61 | Resistant cells | M87339 | replication factor C (activator 1) | 106 | 304 |
| | | | 4 (37 kD) | | |
| 62 | Resistant cells | AB018306 | KIAA0763 gene product | 108 | 305 |
| 63 | Resistant cells | AI761647 | Homo sapiens clone IMAGE | 109 | N/A |
| | | | 21721 | | |
| 64 | Resistant cells | U19718 | microfibrillar-associated protein 2 | 114 | 310 |
| 65 | Resistant cells | U52840 | sema domain, seven | 115 | 311 |
| | | | thrombospondin repeats (type 1 | | |
| | | | and type 1-like), transmembrane | | |
| | | | domain (TM) and short | | |
| | | | cytoplasmic domain, | | |
| | | | (semaphorin) 5A | | |
| 66 | Resistant cells | AB014557 | KIAA0657 protein | 116 | 312 |
| 67 | Resistant cells | AF038172 | hypothetical protein FLJ11149 | 117 | N/A |
| 68 | Resistant cells | U65676 | Hermansky-Pudlak syndrome | 118 | 313 |
| 69 | Resistant cells | U14971 | ribosomal protein S9 | 119 | 314 |
| 70 | Resistant cells | X74331 | primase, polypeptide 2A (58 kD) | 120 | 315 |
| 71 | Resistant cells | D16815 | nuclear receptor subfamily 1, | 121 | 316 |
| | | | group D, member 2 | | |
| 72 | Resistant cells | M14333 | Homo sapiens cDNA FLJ32137 | 122 | 317 |
| | | | fis, clone PEBLM2000479, | | |
| | | | highly similar to PROTO- | | |
| | | | ONCOGENE TYROSINE- | | |
| | | | PROTEIN KINASE FYN (EC | | |
| | | | 2.7.1.112) | | |
| 73 | Resistant cells | U68111 | protein phosphatase 1, | 96 | 295 |
| | | | regulatory (inhibitor) subunit 2 | | |

TABLE 7

| | | | True Class for | Error for | | | | |
|---|---|---|---|---|---|---|---|---|
| | Predicted | Confidence | BMS-A and | BMS-A and | True Class | Error for | True Class | Error for |
| Cell lines | Class | Score | BMS-D | BMS-D | for BMS-B | BMS-B | for BMS-C | BMS-C |
| WiDr | S | 0.174 | S | | S | | S | |
| SW1417 | S | 0.264 | S | | S | | S | |
| SW403 | S | 0.265 | R | * | S | | S | |
| Caco-2 | S | 0.585 | S | | S | | S | |
| SW837 | R | 0.515 | R | | R | | S | * |
| HT29 | S | 0.827 | S | | S | | S | |
| T84 | R | 0.025 | R | | S | * | S | * |
| CCD-33Co | S | 1.000 | S | | S | | S | |
| LOVO | S | 1.000 | S | | S | | S | |
| CCD-18Co | S | 0.572 | S | | S | | S | |
| LS174T | S | 0.905 | S | | S | | S | |
| HCT15 | R | 0.419 | S | * | S | * | R | |
| CX-1 | S | 0.416 | R | * | R | * | S | |
| Colo-205 | R | 0.756 | R | | R | | R | |
| RKO RM13 | R | 0.696 | R | | R | | R | |
| DLD-1 | R | 0.593 | R | | R | | R | |
| Colo-201 | R | 1.000 | R | | R | | R | |
| HCT-8 | R | 0.760 | R | | R | | R | |
| SK-Co-1 | R | 0.898 | R | | R | | R | |
| MIP | R | 0.722 | R | | R | | R | |
| Colo 320hfr | R | 1.000 | R | | R | | R | |
| LS1034 | R | 0.917 | R | | R | | R | |
| Colo320DM | R | 0.950 | R | | R | | R | |
| HCT116 | R | 0.010 | R | | R | | R | |
| HCT116S542 | R | 1.000 | R | | R | | R | |
| LS180 | R | 0.589 | R | | R | | R | |
| LS 513 | R | 0.795 | R | | R | | R | |
| SW1116 | R | 0.695 | R | | R | | R | |
| SW948 | R | 0.523 | R | | R | | R | |
| SW 480 | R | 1.000 | R | | R | | R | |
| SW620 | R | 0.847 | R | | R | | R | |

TABLE 8

| | | | True Class for | Error for | | | | Error |
|---|---|---|---|---|---|---|---|---|
| | Predicted | Confidence | BMS-A and | BMS-A and | True class | Error for | True class | for |
| Cell lines | Class | Score | BMS-D | BMS-D | for BMS-B | BMS-B | for BMS-C | BMS-c |
| WiDr | S | 0.146 | S | | S | | S | |
| SW1417 | S | 0.522 | S | | S | | S | |
| SW403 | S | 0.506 | R | * | S | | S | |
| Caco-2 | S | 0.679 | S | | S | | S | |
| SW837 | R | 0.260 | R | | R | | S | * |
| HT29 | S | 0.920 | S | | S | | S | |
| T84 | S | 0.230 | R | * | S | | S | |
| CCD-33Co | S | 0.979 | S | | S | | S | |
| LOVO | S | 0.969 | S | | S | | S | |
| CCD-18Co | S | 0.488 | S | | S | | S | |
| LS174T | S | 0.619 | S | | S | | S | |
| HCT15 | R | 0.088 | S | * | S | * | R | |
| CX-1 | S | 0.522 | R | * | R | * | S | |
| Colo-205 | R | 0.950 | R | | R | | R | |
| RKO RM13 | R | 0.409 | R | | R | | R | |
| DLD-I | R | 0.755 | R | | R | | R | |
| Colo-201 | R | 0.870 | R | | R | | R | |
| HCT-8 | R | 0.823 | R | | R | | R | |
| SK-Co-1 | R | 0.817 | R | | R | | R | |
| MIP | R | 0.781 | R | | R | | R | |
| Colo 320hfr | R | 0.530 | R | | R | | R | |
| LS1034 | R | 0.815 | R | | R | | R | |
| Colo320DM | R | 0.675 | R | | R | | R | |
| HCT116 | R | 0.261 | R | | R | | R | |
| HCT116S542 | R | 0.782 | R | | R | | R | |
| LS180 | R | 0.499 | R | | R | | R | |
| LS 513 | R | 0.615 | R | | R | | R | |
| SW1116 | R | 0.677 | R | | R | | R | |
| SW 948 | R | 0.500 | R | | R | | R | |
| SW 480 | R | 1.000 | R | | R | | R | |
| SW620 | R | 0.795 | R | | R | | R | |

TABLE 9

| | | | True Class for | Error for | | | | Error |
|---|---|---|---|---|---|---|---|---|
| | Predicted | Confidence | BMS-A and | BMS-A and | True Class for | Error for | True Class | for |
| Cell lines | Class | Score | BMS-D | BMS-D | BMS-B | BMS-B | for BMS-C | BMS-C |
| WiDr | S | 0.020 | S | | S | | S | |
| SW1417 | S | 0.286 | S | | S | | S | |
| SW403 | S | 0.288 | R | * | S | | S | |
| Caco-2 | S | 0.847 | S | | S | | S | |
| SW837 | S | 0.178 | R | * | R | * | S | |
| HT29 | S | 0.876 | S | | S | | S | |
| T84 | S | 0.344 | R | * | S | | S | |
| CCD-33Co | S | 0.870 | S | | S | | S | |
| LOVO | S | 0.908 | S | | S | | S | |
| CCD-18Co | S | 0.333 | S | | S | | S | |
| LS174T | S | 0.468 | S | | S | | S | |
| HCT15 | R | 0.426 | S | * | S | * | R | |
| CX-1 | S | 0.662 | R | * | R | * | S | |
| Colo-205 | R | 0.498 | R | | R | | R | |
| RKO RM13 | R | 0.402 | R | | R | | R | |
| DLD-1 | R | 0.834 | R | | R | | R | |
| Colo-201 | R | 0.695 | R | | R | | R | |
| HCT-8 | R | 0.300 | R | | R | | R | |
| SK-Co-1 | R | 0.525 | R | | R | | R | |
| MIP | R | 0.878 | R | | R | | R | |
| Colo 320hfr | R | 0.474 | R | | R | | R | |
| LS1034 | R | 0.837 | R | | R | | R | |
| Colo320DM | R | 0.436 | R | | R | | R | |
| HCT116 | R | 0.433 | R | | R | | R | |
| HCT116S542 | R | 0.914 | R | | R | | R | |
| LS180 | R | 0.562 | R | | R | | R | |
| LS 513 | R | 0.726 | R | | R | | R | |
| SW1116 | R | 0.589 | R | | R | | R | |
| SW 948 | R | 0.298 | R | | R | | R | |
| SW 480 | R | 0.861 | R | | R | | R | |
| SW620 | R | 0.515 | R | | R | | R | |

TABLE 10

| Accession | | Highly |
|---|---|---|
| # | Unigene Title | Expressed in |
| AB014558 | cryptochrome 2 (photolyase-like) | Sensitive cells |
| AL031228 | ring finger protein 1 | Sensitive cells |
| M22489 | bone morphogenetic protein 2 | Sensitive cells |
| AB023194 | KIAA0977 protein | Sensitive cells |
| U03688 | cytochrome P450, subfamily I (dioxin- | Sensitive cells |
| | inducible), polypeptide 1 (glaucoma 3, | |
| | primary infantile) | |
| AB026891 | solute carrier family 7, (cationic amino acid | Sensitive cells |
| | transporter, y+ system) member 11 | |
| X60708 | dipeptidylpeptidase IV (CD26, adenosine | Sensitive cells |
| | deaminase complexing protein 2) | |
| K03498 | Human endogenous retrovirus HERV-K22 | Sensitive cells |
| | pol and envelope ORF region | |
| D13413 | heterogeneous nuclear ribonucleoprotein U | Sensitive cells |
| | (scaffold attachment factor A) | |
| M69023 | tetraspan 3 | Sensitive cells |

TABLE 11

| Accession | | Highly |
|---|---|---|
| # | Unigene Title | Expressed in |
| AB014558 | cryptochrome 2 (photolyase-like) | Sensitive cells |
| NM_006979 | HLA class II region expressed gene KE4 | Sensitive cells |
| M22489 | bone morphogenetic protein 2 | Sensitive cells |
| AF009674 | axin | Sensitive cells |
| AB006622 | KIAA0284 protein | Sensitive cells |
| AB023194 | KIAA0977 protein | Sensitive cells |
| U03688 | “cytochrome P450, subfamily I (dioxin- | Sensitive cells |
| | inducible), polypeptide 1 (glaucoma 3, | |
| | primary infantile)” | |
| L40802 | hydroxysteroid (17-beta) dehydrogenase 2 | Sensitive cells |
| Y18483 | “solute carrier family 7 (cationic amino | Resistant cells |
| | acid transporter, y+ system), member 8” | |
| U90902 | Human clone 23612 mRNA sequence | Resistant cells |
| S37730 | insulin-like growth factor binding | Resistant cells |
| | protein 2 (36 kD) | |
| X79067 | “zinc finger protein 36, C3H type-like 1” | Resistant cells |
| D87119 | GS3955 protein | Resistant cells |
| M31682 | “inhibin, beta B (activin AB beta | Resistant cells |
| | polypeptide)” | |
| AC005329 | NADH dehydrogenase (ubiquinone) | Resistant cells |
| | Fe—S protein 7 (20 kD) | |
| | (NADH-coenzyme Q reductase) | |

TABLE 12

| Accession | | Highly |
|---|---|---|
| # | Unigene Title | Expressed in |
| AB014558 | cryptochrome 2 (photolyase-like) | Sensitive cells |
| M22489 | bone morphogenetic protein 2 | Sensitive cells |
| AF009674 | axin | Sensitive cells |
| AB006622 | KIAA0284 protein | Sensitive cells |
| AB023194 | KIAA0977 protein | Sensitive cells |
| U03688 | “cytochrome P450, subfamily I (dioxin- | Sensitive cells |
| | inducible), polypeptide 1 (glaucoma 3, | |
| | primary infantile)” | |
| L40802 | hydroxysteroid (17-beta) dehydrogenase 2 | Sensitive cells |
| X77909 | nuclear factor of kappa light polypeptide | Sensitive cells |
| | gene enhancer in B-cells inhibitor-like 1 | |
| U19775 | mitogen-activated protein kinase 14 | Sensitive cells |
| U51903 | IQ motif containing GTPase activating | Sensitive cells |
| | protein 2 | |
| X60708 | “dipeptidylpeptidase IV (CD26, adenosine | Sensitive cells |
| | deaminase complexing protein 2)” | |
| M69023 | tetraspan 3 | Sensitive cells |
| AF155654 | Human putative ribosomal protein S1 | Sensitive cells |
| | mRNA | |
| Y18483 | “solute carrier family 7 (cationic amino acid | Resistant cells |
| | transporter, y+ system), member 8” | |
| U34994 | “protein kinase, DNA-activated, catalytic | Resistant cells |
| | polypeptide” | |
| U90902 | Human clone 23612 mRNA sequence | Resistant cells |
| S37730 | insulin-like growth factor binding protein 2 | Resistant cells |
| | (36 kD) | |
| X79067 | “zinc finger protein 36, C3H type-like 1” | Resistant cells |
| M31682 | “inhibin, beta B (activin AB beta | Resistant cells |
| | polypeptide)” | |
| AC005329 | NADH dehydrogenase (ubiquinone) Fe—S | Resistant cells |
| | protein 7 (20 kD) (NADH-coenzyme | |
| | Q reductase) | |
| M34182 | “protein kinase, cAMP-dependent, catalytic, | Resistant cells |
| | gamma” | |
| AF007156 | KIAA0751 gene product | Resistant cells |
| M14333 | “Homo sapiens cDNA FLJ32137 fis, clone | Resistant cells |
| | PEBLM2000479, highly similar to PROTO- | |
| | ONCOGENE TYROSINE-PROTEIN | |
| | KINASE FYN (EC 2.7.1.112)” | |
| X06745 | “polymerase (DNA directed), alpha” | Resistant cells |
| U84570 | chromosome 21 open reading frame 2 | Resistant cells |

TABLE 13

| | |
|---|---|
| SEQ ID | SEQ ID |

| | | |
|---|---|---|
| DNA | NO: | NO: |

| | | | |
|---|---|---|---|
| Seq | Accession | Forward | Forward |

| | | | | | |
|---|---|---|---|---|---|
| # | # | Forward Primer | Primer | Reverse Primer | Primer |

| | | | | | |
|---|---|---|---|---|---|
| 1 | AB014558 | CTGAACCCTTTGGGAAAGAAC | 391 | AAGCGCTTGTATGTAAGGGGT | 592 |
| 2 | NM_006979 | AGGCTTAGACCTGCGTGTGT | 392 | CTGTCCACTGCTCCTCCTTC | 593 |
| 3 | M22489 | GCAGTTTCCATCACCGAATTA | 393 | ATCAAAACTTTCCCACCTGCT | 594 |
| 4 | AB023194 | AATCCACTGCTTTCATCATGG | 394 | TGTTCAGCTGACAACAGATCG | 595 |
| 5 | U03688 | AACTGTCCATCAGGTGAGGTG | 395 | TTCATTGGGCCCTTTAAGTCT | 596 |
| 6 | M88458 | CTTTGAGGGCTTCTTTGACCT | 396 | TTATGCTGGCAAACTGAGCTT | 597 |
| 7 | L13463 | GCCCAGAAAAGGGTATACAGC | 397 | CTGGGCTCCCTTTTACATTTC | 598 |
| 8 | U21551 | GTGTACCGGAGAAGGAGGATC | 398 | TTATTGGGGTCTGGTTTTTCC | 599 |
| 9 | AF000560 | TATTAGGGCCCGTTCACTTCT | 399 | CCTCTGCAGTTCTCTCCATTG | 600 |
| 10 | AF102265 | TGATCATGTCATGTTTCGCAT | 400 | CATTTGCTAAACAGGTGGCAT | 601 |
| 11 | X06272 | ATGGCACCTCTCCCTAGGATA | 401 | CTGATGCTTTGGGGTAAACAA | 602 |
| 12 | L40802 | ACAAGTGGCATTGGACTCATC | 402 | CAGTTTCCCAGTTTCCCTTTC | 603 |
| 13 | X13916 | GATTGCCTGGACAACAGTGAT | 403 | ACAAGTGGCATTGGACTCATC | 604 |
| 14 | AF009674 | AAGAGCTTCATAAAGGGCTGC | 404 | TGGTCACTACAGACTTTGGGG | 605 |
| 15 | M73077 | GTCGTGACTCCAGAGAAGCC | 405 | AGGTCCAGGTTGTGGTCTTG | 606 |
| 16 | U15655 | TGTAGTGATGGCACGTCAGAG | 406 | GGGATAGACTCGGAAGACACC | 607 |
| 17 | AB014520 | AAGGCCATTCTGAGTATCCGT | 407 | TGGTCTTCCAGATGTGTAGGG | 608 |
| 18 | M58603 | CAGGTCCAGGGTATAGCTTCC | 408 | TTTGTCACAACCTTCAGGGTC | 609 |
| 19 | X76104 | AAGAACCGAGAAGGAGAGACG | 409 | TTACCTCCATCTGACACCGTC | 610 |
| 20 | AI659108 | CACTGTCACAACCCCAATTTC | 410 | ACCACTGTACGGAATGTGAGG | 611 |
| 21 | U72649 | AGAGTGAAAAGGCCTCTCCTG | 411 | CCTTCCATCCTAACCCCAATA | 612 |
| 22 | M64571 | TACGGTATGTCTCCCTGCAAC | 412 | CCTTGGCTAGCTCTAAGGGAA | 613 |
| 23 | X77909 | GTGGGAGCGAAAGTTGTAACA | 413 | TTTGAGATGTGGAACCAGGTC | 614 |
| 24 | M34064 | GGGTAATCCTCCCAAATCAAA | 414 | TCCATACCACAAACATCAGCA | 615 |
| 25 | AL050345 | GGTTGGCAATAGAAGGTGACA | 415 | GAGCACCAAAAAGCTCATCAG | 616 |
| 26 | AB006622 | TACACCTCCACCACTCAGACC | 416 | GAAACCATAAGGGTCAGGCTC | 617 |
| 27 | AB029027 | AAGTATCACGAGAAGCAGGCA | 417 | CAGACAAGAGGCATCTTGAGG | 618 |
| 28 | U51903 | GCTGCAGTGGACCATATCAAT | 418 | CACAGCAATCAAAAGCTCCTC | 619 |
| 29 | AF041259 | AAAAACAAACCGATGTTGCTG | 419 | GGCATCTCCTTAAGCTGCTTT | 620 |
| 30 | AB026891 | ACCTTCCAGAAATCCTCTCCA | 420 | ACCTGGCAAAACTGAGGAAAT | 621 |
| 31 | AB007960 | GGGGCCATTGCTATAAATCAT | 421 | GGGCAAGAACTGTGTGCATAT | 622 |
| 32 | D63390 | TGAAACCAGAAGGGGACTTTT | 422 | CATAATCCACAGAAAGGCCAA | 623 |
| 33 | L10678 | TACCAACGGTTTGACTCTTGG | 423 | ACCTTGACTCTTTGTCCGGAT | 624 |
| 34 | X60708 | AAAAACACAGCAAGGGTGATG | 424 | TCTTTTAACAGGGCAAGCTGA | 625 |
| 35 | Y15521 | AATACTCTCTGCACGGCTTCA | 425 | GGTAGTACGCATCCTGGAACA | 626 |
| 36 | AI038821 | ATCCAATAATTGGGTGGGATC | 426 | AGGCTGTGCACAGACTGTCTT | 627 |
| 37 | X84740 | CAACACGAAGACCCAGATCAT | 427 | AAATGCGACTGAAAAGCTTCA | 628 |
| 38 | M23115 | AACATGAAACAGTTCATCCGC | 428 | GCAGCTGAACAGGAATCAAAG | 629 |
| 39 | U79287 | GCTCATGCTCCTGTACTCGTC | 429 | CCATGCCAGAAACTTGTTGTT | 630 |
| 40 | Y12781 | CTCTTCCTTCTGTTGCTCCCT | 430 | CGTGGGAGATTGTGTCTTCAT | 631 |
| 41 | K03498 | ACAGATGAAGTTGCCATCCAC | 431 | AGCTGCAAGCAGCATACTCTC | 632 |
| 42 | AF030335 | TACTGGTGGTTGAGTTCCTGG | 432 | CAGCTGGACAGAGAAGACCAC | 633 |
| 43 | X93209 | GATGCAATTGACCGTGAAGTT | 433 | TCTAGCAAGGCTTCCAAACAA | 634 |
| 44 | AF068744 | GACAGCGAAGGAGACTCGTTT | 434 | TGAAACAGAATCTGGACCCTG | 635 |
| 45 | AF072247 | TCTGTCCCAGCTCCTTGAGACT | 435 | GACGTGCCTCGACTGTGTTA | 636 |
| 46 | U41344 | TTCCACCCAGTTGAAAGACAC | 436 | GAGAGAAGGGGACACCAAGTC | 637 |
| 47 | D13413 | GGAAGAGGAGGAGGAAGGAAT | 437 | ATCTTCCCCTTCCTGGAAAC | 638 |
| 48 | M69023 | CTGGCATTTGAGCTATTCAGC | 438 | TCAACTATGCATAGGTTCCGC | 639 |
| 49 | J04599 | CTGAAGTCTGTGCCCAAAGAG | 439 | ATCTTGTTGTTCACCAGGACG | 640 |
| 50 | U79267 | GTCAGCGAGATGGTGAAGAAG | 440 | CAGACAAAGACAAAGGCTTGC | 641 |
| 51 | AF155654 | AGAAGGCTGAGGAGAAAGCC | 441 | CTGTGAACCACCGATCTCCT | 642 |
| 52 | X12794 | CAGGACTCTGGCTTCTCTCCT | 442 | CTGTCCTAGGATTGGACCCTC | 643 |
| 53 | U51166 | TTTCAGTGGCATTCCTAATGG | 443 | CTGCAGCATTTAAGCAGAGCT | 644 |
| 54 | L07261 | GTGACTGCATCCAGTTTGGTT | 444 | TGCAGCATAAATTGCAGAGTG | 645 |
| 55 | U97188 | AAACCATGTGATTTGCCTCTG | 445 | GCATTTTCTTTACGGTGGACA | 646 |
| 56 | L37033 | ATCCTCAACCCCATCCAGTAC | 446 | CAGCTCACTCAGGTCATCCTC | 647 |
| 57 | Y09846 | ACCTCATCAGCTACCACATGG | 447 | AGGGCATCTTCTGGAAGAGAG | 648 |
| 58 | AF093420 | ATCTCCTGTCTGGTCCGAGAG | 448 | TGCTGATTTGACCTTGAGCTT | 649 |
| 59 | U19775 | CGAGCTGTTGACTGGAAGAAC | 449 | TCGGCATCTGAGTCAAAGACT | 650 |
| 60 | J04027 | TAAAGCAGTTATGTGGGGACG | 450 | AGGGAAGCGAGTGTATCCATT | 651 |
| 61 | Y18483 | CATCAACTACGTGGGCTTCAT | 451 | CTCTGACCACAGGCTGAAGAC | 652 |
| 62 | U57352 | AGGATGAGTACCTGCCCATCT | 452 | TATGTGAGCCTCTGCTCCTGT | 653 |
| 63 | U34994 | CAGAAACGATCAACACGGAAT | 453 | GGTCTAACATGCCGTTCAAAA | 654 |
| 64 | X79067 | TTGCAAAGGCATCTTCTCAGT | 454 | CTGCCTTTGCTTTTTCTTGTG | 655 |
| 65 | AB011535 | TCTAGAGCAGTGACCCTGGAA | 455 | AGTTGAGCCAGCACAGTCAAT | 656 |
| 66 | U90902 | ACTGTACCCTTCCCTCTTCCA | 456 | GACCAGCCATAGACCAAAACA | 657 |
| 67 | AB009282 | GAACTGTGGCTTGTGATCCAT | 457 | TCGGATGGATATCACCAATGT | 658 |
| 68 | AJ001685 | TCAGGCTTCCTAAAAGTTGCA | 458 | TTTCAACCTCCCTTAGGCATT | 659 |
| 69 | S37730 | TCCCTCGCACATTCAGATAAC | 459 | TAACCACAGCCCTACTCCCTT | 660 |
| 70 | U37518 | AGAGAAGGAAGGGCTTCAGTG | 460 | ATCTGCTTCAGCTCGTTGGTA | 661 |
| 71 | AC005329 | CTCCTTCTGCTGACATTGGAG | 461 | GCAATGTCTGAAAACACGGTT | 662 |
| 72 | AB009426 | TGTGGCTTGGAGATGAATAGG | 462 | TTTTGGGGTACCTTGTGAACA | 663 |
| 73 | X70340 | GAATGACTCAAATGCCCAAAA | 463 | AAGCCTGGTAAATCAATGGCT | 664 |
| 74 | U81561 | GTATGACCGAGGAGTCCCTTC | 464 | ATGTCGATCAGGACGTAGGTG | 665 |
| 75 | X70040 | CACACCCCTGCCTATTCTGTA | 465 | GTGGCACACAGGATTCATCTT | 666 |
| 76 | AB000449 | ATGGCCTTGCTTATCGGTACT | 466 | CCATTGGATCATGCAATAACC | 667 |
| 77 | D87119 | AAGGAACAGTTGGCCAAGAAT | 467 | GTCTGTGTGCACCGAATTTTT | 668 |
| 78 | X06745 | AATGCTACCTGTGGTCGAATG | 468 | TCTTTCCAGGTGTGTTCCAAC | 669 |
| 79 | X78817 | AACAAGACTCTGAAGGCGACA | 469 | GTCTGAGCTGGTGGACTTGAG | 670 |
| 80 | AF070530 | ACCACATGTGGAACCAGAGAG | 470 | TGACCTCATCTTCCACTGTCC | 671 |
| 81 | L43821 | GACAGGCCATGGCTACGTATA | 471 | ACTGAAAACACAGGGCCTTTT | 672 |
| 82 | AF007156 | CCCACCATAGAATTTCTGCAA | 472 | AGACTGAAGCCTGTCCTGTCA | 673 |
| 83 | AB014566 | AGGAGGAAGAAGAACGTCGAG | 473 | GTCAAACACTTCTCCTGAGCG | 674 |
| 84 | U71364 | ATTGTTGATGCCTTCCAACAG | 474 | CCTTCTTCATTCACCTCCACA | 675 |
| 85 | U93305 | TCTACCTGCCACCCCTACTTT | 475 | GCCTGTGCATCTATTCTCTGC | 676 |
| 86 | AB006626 | AGAACGGTCTTGGGACTTGTT | 476 | CAGAGGGCTATGCAGAGAATG | 677 |
| 87 | M31682 | GCAATGACCGTTTGACTGTTT | 477 | ATTTAGCCCCCTCTTCTCTCC | 678 |
| 88 | AF031824 | AAGACCACAGCCATGACAAAC | 478 | TGTTAGGAGGTGCTACCATGC | 679 |
| 89 | AF035299 | GAGGGCTCTACCTGAGAAGGA | 479 | GGATTGTCCTTCCCTTGACTC | 680 |
| 90 | X82207 | TAACCCTTTTTGGTCTTGGCT | 480 | CATGTCCTGTGTAGAGGGGAA | 681 |
| 91 | U84570 | GTTAGGTACTGGCTAACCGGG | 481 | AAATCCTCCCTTTAAGAGCCC | 682 |
| 92 | AA873266 | GGGAAAGTCCAGGTGGTAGAG | 482 | AAGATTGCCTTCTGCAAGTCA | 683 |
| 93 | X90976 | CCATGTCTGACCTGCAAAAAT | 483 | AGGCTGGTTTTGAGTTGGAAT | 684 |
| 94 | D89377 | CTCCAGCTTCAGTCTCCCTTT | 484 | GGTCTTCCTTAGGACAGGTGG | 685 |
| 95 | M57730 | CTGGAACAGTTCAAATCCCAA | 485 | CAGCTGGTACTCCTCATGCTC | 686 |
| 96 | U68111 | CAAGTGACCAACAGCAAAACA | 486 | TGTGAAGAACAAGAAGCAACG | 687 |
| 97 | L07540 | AGTCAGACATTGCCAACATCC | 487 | CTCAATGTCTGCCATTTTGGT | 688 |
| 98 | M65066 | AGAGTGGGTGACCAACATCAG | 488 | GAACTCCTCGTACATCTTGCG | 689 |
| 99 | M34182 | AAGCCCAGATATTTGGAGGAA | 489 | GTTTAAAACAGGCAGAAGGGG | 690 |
| 100 | L34059 | CCATGGAGGTCTTCAGCATTA | 490 | TGTCATTCATGTCGATGACGT | 691 |
| 101 | L25665 | ATCGATACCGACTGCATTTTG | 491 | TCGAGGAAAGTCCAGAACTGA | 692 |
| 102 | AL050290 | GGACTCCGGAAGGTTACAGTC | 492 | AACCAACAATGCTGTGTCCTC | 693 |
| 103 | X67325 | ACTCTCCGGATTGACCAAGTT | 493 | CTGGCATGGTTCTCTTCTCTG | 694 |
| 104 | AA595596 | TCACCACAGCTGAAGGAAATT | 494 | TGGGAGTACAGTGCCATTAGG | 695 |
| 105 | AF003837 | CCTGTAACATAGCCCGAAACA | 495 | AGTTGTCTCCATCCACACAGG | 696 |
| 106 | M87339 | TTCCCTGGGTGGAAAAATATC | 496 | CAGGTGGTCCGTAAAACAAGA | 697 |
| 107 | AI813532 | TCTGACATCTTGATTCCAGGG | 497 | GGCAGGGTGATAAATTGTTGA | 698 |
| 108 | AB018306 | AGCGGAAAATGAAGCTAGAGG | 498 | GATCCGTTCATAGATCCCCAT | 699 |
| 109 | AI761647 | GAAAAGACCCAAGGTTTCTGG | 499 | CCAAAGGCTGGTAGGAGATTC | 700 |
| 110 | X80507 | CAACTGCAGATGGAGAAGGAG | 500 | GACACTGGATTTTGAGTCCCA | 701 |
| 111 | Y16241 | TGGTTTGGCAACAGGTATCTC | 501 | GAAAGTCAGGTGCATGCTCTC | 702 |
| 112 | D67031 | TGAATATGGGTTCCCATCAAA | 502 | GTGCCTAGGCTTCTCTCGAAT | 703 |
| 113 | J05581 | CCAGTCTCCTTTCTTCCTGCT | 503 | CAGTAGAGCTGGGCACTGAAC | 704 |
| 114 | U19718 | CTCTTCCTGCTATTCCTGCCT | 504 | TAGTCTGGGTTGTCGATCTGG | 705 |
| 115 | U52840 | GTTCATTCTGGTGCATGAGGT | 505 | CTGCTTGAGGTCTGATTCAGG | 706 |
| 116 | AB014557 | GTTTGAGTGCAGGACAGAAGG | 506 | ACACTCGGAAGTTATGGCATG | 707 |
| 117 | AF038172 | TTACAGCTTGAGGGAAAAGCA | 507 | GGCCCAAAGCATCTGTAATCT | 708 |
| 118 | AB014529 | CTGAAGCCATTGAAAAAGCTG | 508 | CTAACAGCATGCCCAACATTT | 709 |
| 119 | U65676 | AGGCTTTCTCCAAAAGTGAGC | 509 | TCAGAAAGTTCAGCCGGTAGA | 710 |
| 120 | U14971 | TTGACTTCTCTCTGCGCTCTC | 510 | TGTTTATTTGGCAGGAAAACG | 711 |
| 121 | X74331 | CCAAACCAAGTGTCCAGAAAA | 511 | CAGGCTATTGAGGAAAAAGGG | 712 |
| 122 | D16815 | GTGAATGCAGGAGGTGTGATT | 512 | CCTTCAATATTGGCGAGATCA | 713 |
| 123 | M14333 | CCTCTGTGAAGCATTCGAGAC | 513 | GGATTGTTGGCACTGGAGTAA | 714 |
| 124 | M28668 | ATTATCACCAGCACCAGTTCG | 514 | GGGTTGACATAGGTGCTTGAA | 715 |
| 125 | W29065 | GAGAGAATAACCATCCGGGAC | 515 | AAGATGGGGAGATGTGGAAAC | 716 |
| 126 | AL050025 | CAGGTACGAATTTTGCGGTTA | 516 | TCGCAATCCACTCTCTGACTT | 717 |
| 127 | AF000561 | TACGAGTGCAACATCTGCAAG | 517 | TCTTCAGGTCGTAGTTGTGGG | 718 |
| 128 | AB008515 | ATGGGAATTGGTCTTTCTGCT | 518 | CACAACAGCCAATGACATCAC | 719 |
| 129 | AJ011736 | GAGGTCCTGGATAGCTCCAAC | 519 | AAGCTTCTGTCCCCTGAAGAG | 720 |
| 130 | J00277 | GCTGAAAGGAAAGCAGATGTG | 520 | GGACTTCCCAGTCTTGTCCTC | 721 |
| 131 | Y10055 | AGGCCTCATTGAGGTGGTACT | 521 | CTTGGACTTCAGCCAGTTGAG | 722 |
| 132 | W26652 | TTTTCCGTTGCAGCTGTTAAT | 522 | CTCACTTAGCGAAAGTGACGG | 723 |
| 133 | M11717 | GACGAGTTTGAGCACAAGAGG | 523 | AGGCCCCTAATCTACCTCCTC | 724 |
| 134 | L32976 | ACCCTGAAGATCACCGACTTT | 524 | GAAGGTGGAGGCCTTGATAAC | 725 |
| 135 | M29893 | CTAGATGGGGAGGAAGTCCAG | 525 | GAGAAAACACAGAGGAACCCC | 726 |
| 136 | S70154 | TGAGATGCCACTGACTGACAG | 526 | CTTGCCATTTTGTGGCTACAT | 727 |
| 137 | D83542 | ACTTCATCAATGATGGCTTGG | 527 | CCCCCAGTCTCTGAGGTAGTC | 728 |
| 138 | Z74615 | GTACATCAGCAAGAACCCCAA | 528 | TGGTAGGTGATGTTCTGGGAG | 729 |
| 139 | M96684 | GACTACGGAGTGGAGGAGGAG | 529 | GCATAAACACGCCGTACTTGT | 730 |
| 140 | D12763 | CCTGTGCCATAAAATGTGCTT | 530 | GGGAGGACGAACAATTTAAGC | 731 |
| 141 | S67070 | AGCCATAGTTGAGCCCTGATT | 531 | GATGCTGCTACCTCTGGAGTG | 732 |
| 142 | X66435 | CTCACCTCAGCATTTAGCAGG | 532 | GTGCCACACCAGTTCTTGAAT | 733 |
| 143 | U25138 | AAGTCATTGCCTGCTCAAGAA | 533 | CAGTTGAGTGGGGACAGGTAA | 734 |
| 144 | D85131 | ACTGGTGAGGTTTGTCCAATG | 534 | GGAGCTTGTACCAAGGGACTC | 735 |
| 145 | M36089 | CAGACAAAGATGAGGCAGAGG | 535 | CTGTGACTGGGGATGTCTTGT | 736 |
| 146 | AC004472 | TCTACTCTCAACTCCAGGGCA | 536 | TGGAGCTGACCTGACAGAGAT | 737 |
| 147 | AB009010 | GAATGTCAAGGCAAAGATCCA | 537 | TGATGGTCTTACCAGTCAGGG | 738 |
| 148 | D26158 | CGCTTTGACAAGAGGATTGAG | 538 | TGACTGGTAGAGGTGGGTGAG | 739 |
| 149 | AL035307 | CTTTGTCATCATTGCCCAACT | 539 | TGGTTCATTTTGGCTCTCATC | 740 |
| 150 | AL043470 | TACACTTCCACAGTCAGCACG | 540 | AAGGACAGGTATGATGATGCG | 741 |
| 151 | U67733 | CAAGTACTACCTTCCTGGGCC | 541 | TGCTCAGACAAAGAATGGCTT | 742 |
| 152 | D28118 | TCACCATCACATCTCCAATGA | 542 | TTTACCAAGGCGGTGATGTAG | 743 |
| 153 | AB020661 | CATTAGGACACGTCATGCCTT | 543 | GAGTTGATCATCGTGGCATTT | 744 |
| 154 | W72186 | TATAGCAACAGCGTGTGCAAG | 544 | TTTCATGCACACACACACATG | 745 |
| 155 | U97067 | ATGAGCACAGAGAACGCATCT | 545 | GTTCTTCAGCGATGCTTTTTG | 746 |
| 156 | AF061261 | CCCAAAAGTTGTCAGGTTGAA | 546 | TTTTAAGTGTGTCGGAGGGTG | 747 |
| 157 | AB007870 | ACATGAATATGGGCACAGAGC | 547 | TCTGCAGCCTTCAGAACAAAT | 748 |
| 158 | AB014519 | CTTAGCAATGAGATGCAAGCC | 548 | GCATTTTTGTGCTGAAGAAGC | 749 |
| 159 | AB011123 | CATTGAGTTGAGCTGGCCTAG | 549 | GCTTGTTTCCCAGAAGCTCTT | 750 |
| 160 | U09578 | TTTCATGGCTGATCAGAGCTT | 550 | TAAAACCCAGCAGTGTTGTCC | 751 |
| 161 | Y17711 | TCTGTGTTAGTCCCTGGGATG | 551 | GCTCACTAAGCACAGGGTCAC | 752 |
| 162 | L13972 | TCCTCTCGGTCATCTTCTCAA | 552 | TGGGTTGTTCTCCCAGTAGTG | 753 |
| 163 | AF055009 | CAAGTACCTGAGTGAGGCCTG | 553 | AGCAAGAACTCCTCTTCTGGG | 754 |
| 164 | AF007155 | TTCCAGAACTTCTCCCTCCAT | 554 | GAGGCACTCAGTCTCCCTTCT | 755 |
| 165 | AB018324 | AGCACCTTATCCGAAGGATGT | 555 | AGTCGCAGAACCTGCTCATTA | 756 |
| 166 | AB023154 | ATGAGGTCCACCACAAGACAG | 556 | AAAGCTTTTCTGGCCTCAGTC | 757 |
| 167 | X78992 | ATCCAGAAACATGTCGACCAC | 557 | GCATGTTGTTCAGGTTGAGGT | 758 |
| 168 | M60299 | CTTGCTCCTTTCTTTTGCATG | 558 | ACTCCACTGAACCCCTCTGTT | 759 |
| 169 | M80482 | TAAAAAGTGCGTGGATGAACC | 559 | GTGAATGCACTCTTCTCTGCC | 760 |
| 170 | U47025 | GGTCAGTGACGAGGTGTTCAT | 560 | GGATCCTCTTCACATGCACAT | 761 |
| 171 | Y10032 | AGGATGGGTCTGAACGACTTT | 561 | AAGGGTTGGCATTCATAAGCT | 762 |
| 172 | U57057 | GGGAACTGTTTAAGGGTGAGC | 562 | CACGGAGTCGTAGCAGTTCTC | 763 |
| 173 | X90392 | CTCATTTGTCCCAACAGCATT | 563 | TGAGTTAGGGGTGTTTGATGC | 764 |
| 174 | AA418437 | GAGACAGGGCTGTGTTCTGAG | 564 | AGCAGTAGCAGTGAGAGCGAG | 765 |
| 175 | X84373 | TCCTCCTTGCGTAGAAGTTGA | 565 | ATGGTTTTCTGTGGTGAGTGC | 766 |
| 176 | XM_212189 | TGTTCACTTTTGTTGCGGAG | 566 | CTGCATGGAGAAGATGACGA | 767 |
| 177 | AF109134 | GGGAGGTCGAGGTGTTTAAAA | 567 | GGAAGCGCTTCTGGTAGTTCT | 768 |
| 178 | M64788 | ATTTGGGTTCTGCTGTGTGAC | 568 | AAGGCAGGTGCATGTAATCAC | 769 |
| 179 | U43368 | AAGAGGGGTCACATACCAGCT | 569 | GGGGTCACAGTTCTTGTACCA | 770 |
| 180 | X13293 | ACCAGTCTGTCCTTCCTGGAT | 570 | GCTCCAATGTGTCCTGTTTGT | 771 |
| 181 | AB014511 | CTCAGTATCGTCTCCAGCCAG | 571 | ATAAAGCGGGATCACACCTCT | 772 |
| 182 | AB014585 | CCGTGAGGAGAGGACAGAAG | 572 | CACTTCTGGAACAGGTGGGT | 773 |
| 183 | AB011114 | CGATTCCATCATGTCACTGTG | 573 | CAGCAGCATGTAGTGTTTCCA | 774 |
| 184 | U80040 | CACTTCCGTGTTCCCTTACAA | 574 | TAGTTGGTCATAATGGCAGCC | 775 |
| 185 | M64174 | TGAAGCCTGAGAGTGGAGGTA | 575 | CTCCGTCTTCTGTGCAGATTC | 776 |
| 186 | Y00971 | ACTTGTGGCCAATATGCTGTC | 576 | GCTCCGCATACAAATTATCCA | 777 |
| 187 | AL050065 | CCTTTCATGTCTCCCACTGAA | 577 | GCCCACAGATACTTGACCGTA | 778 |
| 188 | AF029670 | TCCGAGCTTAGCAAAGAAGTG | 578 | CCTGCTCAAGAAGTTCCAGTG | 779 |
| 189 | X63629 | TGTTGAATAAGCCACTGGACC | 579 | GTAAACTTGGGCTTGTGGTCA | 780 |
| 190 | AB028957 | TGTTGTGACGTTGAAAATCCA | 580 | AGGGCATTCTTTGGCTAATGT | 781 |
| 191 | X74837 | TTTCGAAAGAAAGCAGTGGAA | 581 | GGGTTTCCTGATAAGTGGCTC | 782 |
| 192 | AB018273 | ACGACGTTGGATGAAGAAATG | 582 | CCTCCTCAGGCTTCAGAAGTT | 783 |
| 193 | Y15227 | AAATACGGGTCCTGCTTAGGA | 583 | GAGGCTTAAGTGCGATAACCC | 784 |
| 194 | Y12661 | TCTATTTTTCAGTCTCCGGCA | 584 | TACCGGCTCTTTATGCTCAGA | 785 |
| 195 | AL049365 | TTCTTGGTCTTGGAACTCCCT | 585 | TTAAAGTGCCAGTATCGGTGG | 786 |
| 196 | Y11395 | CAGGATCAGCTTCCTCCTTCT | 586 | TTCACAAGAAAGGCAGAGCAT | 787 |
| 197 | NM_004713 | GGTGACTCGAGCAGTGATGA | 587 | TTTGGGGTTGAAGGTGAGAC | 788 |
| 198 | X76029 | TGGAGGAGCTTTGCTTTATGA | 588 | CAACATTTGACTTGCCCAACT | 789 |
| 199 | AF045583 | TCTTCTTGCAGCTAGAAAGCG | 589 | CAGATGCCACGGTCATAAACT | 790 |
| 200 | X85545 | ACCTTTTCTTCACGTGGAGGT | 590 | TGAGAAGCACAGGCTGAAAAT | 791 |
| 201 | X12534 | CAGAGCTTCCAGGACATCAAG | 591 | GAAGTTTCCATAAAGGGGCAG | 792 |

The contents of all patents, patent applications, published PCT applications and articles, books, references, reference manuals, abstracts and internet websites cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

What is claimed is:

1. A predictor set comprising a plurality of polynucleotides whose expression pattern is predictive of the response of cells to treatment with a compound that modulates protein tyrosine kinase activity or members of the protein tyrosine kinase pathway.

2. The predictor set according to claim 1 wherein the polynucleotides are selected from the group consisting of:
a.) the polynucleotides provided in Table 3;
b.) the sensitive predictor polynucleotides provided in Table 3;
c.) the resistant predictor polynucleotides provided in Table 3;
d.) the polynucleotides provided in Table 4;
e.) the sensitive predictor polynucleotides provided in Table 4;
f.) the resistant predictor polynucleotides provided in Table 4;
g.) the polynucleotides provided in Table 5;
h.) the sensitive predictor polynucleotides provided in Table 5;
i.) the resistant predictor polynucleotides provided in Table 5;
j.) the polynucleotides provided in Table 6;
k.) the sensitive predictor polynucleotides provided in Table 6; and
l.) the resistant predictor polynucleotides provided in Table 6;

3. The predictor set according to claim 2 wherein the plurality of polynucleotides comprise the number of polynucleotides selected from the group consisting of:
a.) at least about 5 polynucleotides;
b.) at least about 10 polynucleotides;
c.) at least about 15 polynucleotides;
d.) at least about 20 polynucleotides;
e.) at least about 25 polynucleotides; and
f.) at least about 30 polynucleotides.

4. The predictor set according to claims 3 wherein the plurality of polynucleotides comprise a member of the group consisting of:
a.) the polynucleotides provided in Table 10;
b.) the sensitive predictor polynucleotides provided in Table 10;
c.) the resistant predictor polynucleotides provided in Table 10;
d.) the polynucleotides provided in Table 11;
e.) the sensitive predictor polynucleotides provided in Table 11;
f.) the resistant predictor polynucleotides provided in Table 11;
g.) the polynucleotides provided in Table 12;
h.) the sensitive predictor polynucleotides provided in Table 12; and
i.) the resistant predictor polynucleotides provided in Table 12.

5. The predictor set according to claim 4 wherein the protein tyrosine kinases are selected from the group consisting of: Src, Fgr, Fyn, Yes, Blk, Hck, Lck and Lyn, Bcr-abl, Jak, PDGFR, c-kit and Ephr.

6. The predictor set according to claim 5 wherein the compound is selected from the group consisting of:
a.) antisense reagents directed to said polynucleotides;
b.) antibodies directed against polypeptides encoded by said polynucleotides; and
c.) small molecule compounds.

7. The predictor set according to claim 5 wherein the compound is selected from the group consisting of:
a.) BMS-A;
b.) BMS-B;
c.) BMS-C; and
d.) BMS-D.

8. The predictor set according to claim 1 wherein said cells are colon cancer cells.

9. A predictor set comprising a plurality of polypeptides whose expression pattern is predictive of the response of cells to treatment with compounds that modulate protein tyrosine kinase activity or members of the protein tyrosine kinase pathway.

10. The predictor set according to claim 9 wherein the polypeptides are selected from the group consisting of:
a.) the polypeptides provided in Table 3;
b.) the sensitive predictor polypeptides provided in Table 3;
c.) the resistant predictor polypeptides provided in Table 3;
d.) the polypeptides provided in Table 4;
e.) the sensitive predictor polypeptides provided in Table 4;
f.) the resistant predictor polypeptides provided in Table 4;
g.) the polypeptides provided in Table 5;
h.) the sensitive predictor polypeptides provided in Table 5;
i.) the resistant predictor polypeptides provided in Table 5;
j.) the polypeptides provided in Table 6;
k.) the sensitive predictor polypeptides provided in Table 6; and
l.) the resistant predictor polypeptides provided in Table 6.

11. The predictor set according to claim 10 wherein the plurality of polypeptides comprise the number of polypeptides selected from the group consisting of:
a.) at least about 5 polypeptides;
b.) at least about 10 polypeptides;
c.) at least about 15 polypeptides;
d.) at least about 20 polypeptides;
e.) at least about 25 polypeptides; and
f.) at least about 30 polypeptides.

12. The predictor set according to claims 11 wherein the plurality of polypeptides comprise a member of the group consisting of:
a.) polypeptides provided in Table 10;
b.) the sensitive predictor polypeptides provided in Table 10;
c.) the resistant predictor polypeptides provided in Table 10;
d.) the polypeptides provided in Table 11;
e.) the sensitive predictor polypeptides provided in Table 11;
f.) the resistant predictor polypeptides provided in Table 11;
g.) the polypeptides provided in Table 12;
h.) the sensitive predictor polypeptides provided in Table 12; and
i.) the resistant predictor polypeptides provided in Table 12.

13. The predictor set according to claim 12 wherein the protein tyrosine kinases are selected from the group consisting of: Src, Fgr, Fyn, Yes, Blk, Hck, Lck and Lyn, Bcr-abl, Jak, PDGFR, c-kit and Ephr.

14. The predictor set according to claim 13 wherein the compound is selected from the group consisting of:
a.) antisense reagents directed to polynucleotides encoding said polypeptides;
b.) antibodies directed against said polypeptides; and
c.) small molecule compounds.

15. The predictor set according to claim 13 wherein the compound is selected from the group consisting of:
a.) BMS-A;
b.) BMS-B;
c.) BMS-C; and
d.) BMS-D.

16. The predictor set according to claim 9 wherein said cells are colon cancer cells.

17. A method for predicting whether a compound is capable of modulating the activity of cells, comprising the steps of:
a.) obtaining a sample of cells;
b.) determining whether said cells express a plurality of markers; and
c.) correlating the expression of said markers to the compounds ability to modulate the activity of said cells.

18. The method according to claim 17 wherein the plurality of markers are polynucleotides.

19. The method according to claim 18 wherein the polynucleotides are selected from the group consisting of:
a.) the polynucleotides of claim 1;
b.) the polynucleotides of claim 2;
c.) the polynucleotides of claim 3; and
d.) the polynucleotides of claim 4.

20. The method according to claim 19 wherein the compounds are a member of the group consisting of:
a.) the compounds according to claim 5;
b.) the compounds according to claim 6; and
c.) the compounds according to claim 7.

21. The method according to claim 18 wherein the cells are colon cancer cells.

22. The method according to claim 17 wherein the plurality of markers are polypeptides.

23. The method according to claim 22 wherein the polypeptides are selected from the group consisting of:
a.) the polypeptides of claim 9;
b.) the polypeptides of claim 10;
c.) the polypeptides of claim 11; and
d.) the polypeptides of claim 12.

24. The method according to claim 23 wherein the compounds are a member of the group consisting of:
d.) the compounds according to claim 13;
e.) the compounds according to claim 14; and
f.) the compounds according to claim 15.

25. The method according to claim 19 wherein the cells are colon cancer cells.

26. A plurality of cell lines for identifying polynucleotides and polypeptides whose expression levels correlate with compound sensitivity or resistance of cells associated with a disease state.

27. The plurality of cell lines according to claim 26 wherein said cell lines are colon cancer cell lines.

28. The plurality of cell lines according to claim 27 wherein said cell lines comprise one or more cell lines provided in Table 1.

29. A method of identifying polynucleotides and polypeptides that predict compound sensitivity or resistance of cells associated with a disease state, comprising the steps of:
a.) subjecting the plurality of cell lines according to claim 28 to one or more compounds;
b.) analyzing the expression pattern of a microarray of polynucleotides or polypeptides; and
c.) selecting polynucleotides or polypeptides that predict the sensitivity or resistance of cells associated with a disease state by using said expression pattern of said microarray.

30. The method according. to claim 23 wherein the compounds are a member of the group consisting of:
a.) the compounds according to claim 5;
b.) the compounds according to claim 6;
c.) the compounds according to claim 7;
d.) the compounds according to claim 13;
e.) the compounds according to claim 14; and
f.) the compounds according to claim 15

31. The method according to claim 29 wherein said disease is colon cancer.

32. A method for predicting whether an individual requiring treatment for a disease state, will successfully respond or will not respond to said treatment comprising the steps of:
a.) obtaining a sample of cells from said individual;
b.) determining whether said cells express a plurality of markers; and
c.) correlating the expression of said markers to the individuals ability to respond to said treatment.

33. The method according to claim 32 wherein the plurality of markers are polynucleotides.

34. The method according to claim 33 wherein the polynucleotides are selected from the group consisting of:
a.) the polynucleotides of claim 1;
b.) the polynucleotides of claim 2;
c.) the polynucleotides of claim 3; and
d.) the polynucleotides of claim 4.

35. The method according to claim 34 wherein the compounds are a member of the group consisting of:
a.) the compounds according to claim 5;
b.) the compounds according to claim 6; and
c.) the compounds according to claim 7.

36. The method according to claim 33 wherein the disease state is colon cancer.

37. The method according to claim 34 wherein the plurality of markers are polypeptides.

38. The method according to claim 37 wherein the polypeptides are selected from the group consisting of:
a.) the polypeptides of claim 9;
b.) the polypeptides of claim 10;
c.) the polypeptides of claim 11; and
d.) the polypeptides of claim 12.

39. The method according to claim 38 wherein the compounds are a member of the group consisting of:
a.) the compounds according to claim 5;
b.) the compounds according to claim 6; and
c.) the compounds according to claim 7.

40. The method according to claim 37 wherein the disease state is colon cancer.
